(19) United States
(12) Patent Application Publication
(10) Pub. No.: US 2023/0017907 A1
(43) Pub. Date: Jan. 19, 2023

(54) METHOD FOR CONTINUOUS PROTEIN RECOVERING

(71) Applicant: TAKEDA PHARMACEUTICAL COMPANY LIMITED, OSAKA (JP)

(72) Inventors: Daniel FLEISCHANDERL, Vienna (AT); Christoph DATTENBOECK, Vienna (AT); Katerina PETRUSHEVSKA-SEEBACH, Vienna (AT); Thomas GATTERNIG, Vienna (AT); Martin PURTSCHER, Vienna (AT); Alois JUNGBAUER, Vienna (AT); Hannah ENGELMAIER, Vienna (AT); Nikolaus HAMMERSCHMIDT, Vienna (AT)

(73) Assignee: TAKEDA PHARMACEUTICAL COMPANY LIMITED, OSAKA (KR)

(21) Appl. No.: 17/784,439

(22) PCT Filed: Dec. 10, 2020
(86) PCT No.: PCT/EP2020/085483

§ 371 Date: Jun. 10, 2022

Related U.S. Application Data
(60) Provisional application No. 62/947,209, filed on Dec. 12, 2019.

(51) Int. Cl. C07K 1/30; C07K 14/755; B01L 3/00

(52) U.S. Cl. CPC C07K 1/303; C07K 14/755; B01L 3/502

(57) ABSTRACT

The present invention relates to a method for continuous recovering of a protein from a fluid, comprising precipitating the protein in the fluid and separating the precipitated protein from the fluid. The invention also provides an inclined plate settler that can be used for such continuous protein recovering.

### FIELD OF THE INVENTION

The present invention relates to a method for continuous recovering of a protein from a fluid, comprising precipitating the protein in the fluid and separating the precipitated protein from the fluid. The invention also provides an inclined plate settler that can be used for such continuous protein recovering.

### BACKGROUND

Traditionally, biopharmaceutical processes have been and are still run in (semi-)batch-wise manner. In batch processing all unit operations are performed sequentially, meaning the process moves on to the next step when the previous operation is completed. This course of action requires collection of the product in hold tanks between the individual steps. Consequently, batch production processes are characterized by high residence times during which conditions for the product can at times be non-ideal. Long hold up and residence times are particularly critical for inherently labile products such as enzymes or blood coagulation factors.

The first hybrid processes consisting of a continuous upstream (e.g. perfusion cell-culture, with a batch downstream process) were therefore developed for blood coagulation factors and enzymes. With advances in production cell lines and titers, the bottleneck was shifted from upstream to downstream processing. Therefore, interest in continuous processing of biopharmaceuticals has grown tremendously over the last years. Integrated, continuous processing reportedly offers a list of benefits such as (i) improved product quality, (ii) increased process control and process understanding, (iii) reduced cost of goods (COGs), (iv) smaller equipment size, resulting in reduced footprint, i.e. facility size, and equipment cost, (v) increased productivity and (vi) higher flexibility. Notably, the full potential of continuous processing can only be utilized in a fully integrated, continuous or end-to-end continuous process.

Protein precipitation can be used in the downstream processing of biopharmaceutical production processes. It is often used to capture the target protein, and thereby achieve a significant volume reduction, i.e., a concentration of the target protein. Precipitation can be scaled up linearly, does not require complex equipment and can be performed under non-denaturizing conditions. Moreover, precipitation can in principle be operated continuously, as the only requirements are continuous addition of the precipitant(s) to the process stream and efficient mixing, and depending on the precipitation kinetics, sufficient time for completion of the precipitation needs to be guaranteed.

Despite their recognized potential, examples for continuous precipitation processes for recombinant proteins are rare (cf. references 1 to 3). In one example, a two-stage precipitation process using CaCl₂ to remove DNA and subsequent cold ethanol precipitation to capture the product (mAb) was shown to result in yields of >90% with significant impurity reductions (cf. reference 4). Furthermore, continuous precipitation of various antibodies using Polyethylenglycol (PEG) and subsequent precipitate recovery based on tangential flow filtration was reported (cf. reference 5). A very similar process was developed by for continuous antibody precipitation including integrated continuous precipitate separation and wash (cf. reference 6). Finally, continuous precipitation of impurities using different precipitation methods was shown, though the focus of this study was on demonstrating the wide range of possible applications for a so-called coiled flow invertor reactor (CFIR), rather than on precipitation process development (cf. reference 7). Nevertheless, overall there is an apparent lack of examples for continuous precipitation processes.

In the current invention the protein to be recovered (e.g., a coagulation factor) can be captured, i.e. precipitated, e.g. using calcium phosphate precipitation. At least due to this feature, it differs from the process in reference 4, where one of the main impurities (DNA) is precipitated using calcium ions. In all of references 1 to 7, the product is a monoclonal antibody that is precipitated using either PEG or cold ethanol. Monoclonal antibodies are typically produced at titers in the g/L range. These titers are several orders of magnitude higher than in the production of recombinant blood coagulation factors. One of the advantages of the current invention is that it allows capture of a complex product that is present at very low concentrations.

The main bottleneck of a continuous precipitation process is the solid-liquid separation step. In batch operation, solid-liquid separation can easily be performed by dead end filtration or centrifugation. In the past, the need for centrifugation and associated challenges have hampered the application of precipitation at manufacturing scale (cf. reference 8). When moving to continuous operation, especially centrifugation becomes more challenging. Most semi-continuous centrifuges are operated with periodic discharge of solids, which creates a discontinuous output. Furthermore, difficulties to efficiently re-solubilize precipitate after centrifugation were reported, which were solved by using transmembrane flow filtration for separation (cf. reference 5). However, not all precipitates may be suitable for separation by transmembrane flow filtration. Furthermore, sequential separation and dissolution of the precipitate collected in a transmembrane flow filtration module, also produces a periodic output, similar as in centrifugation.

In view of the above, there is great demand for improved methods for continuous protein recovering, in particular for continuous protein recovering methods comprising protein precipitation and continuous solid-liquid separation (i.e., continuous precipitate-liquid separation).

### DESCRIPTION OF THE INVENTION

The present invention meets the above-described needs and solves the above-mentioned problems in the art:

The present inventors have surprisingly found that a protein can be continuously recovered from a fluid by precipitating the protein and separating the precipitated protein from the fluid. When calcium phosphate is used for protein precipitation, the protein recovering allows to recover relatively large proteins (e.g., coagulation factors) even at very low concentrations. The efficiency of the protein recovering can be further improved by adjusting the pH of the fluid comprising the protein before precipitation, and by using calcium phosphate within defined concentration ranges.

Additionally, the inventors have surprisingly found that the method for continuous recovering of a protein from a fluid is particularly efficient when a plate settler is used for separating the protein precipitate, and when this plate settler is connected to a specially designed bottom section. This specially designed bottom section comprises at least one inlet channel for feeding the fluid comprising the precipitated protein to the plate settler, and at least one collection channel for collecting the settled precipitated protein, wherein the inlet channel and the collection channel are fluidly separated from each other. The fluid separation between inlet channel and collection channel (i.e., the absence of a direct fluid communication) promotes a better control over the behavior of fluid flows in the bottom section. Specifically, turbulences arising from mixtures of fluid being supplied and the descending protein precipitate and/or a descending separated fluid (e.g., comprising the precipitated protein to be separated) in the bottom section are lowered or even avoided. Also, less or no separated protein precipitate is mixed into newly supplied fluid (i.e., precipitate suspension). Thus, the efficiency of the separation process is increased by the bottom section in accordance with the present invention.

Furthermore, the inventors have found that it is particularly advantageous when the bottom section further comprises at least one wash fluid supply channel that is fluidly separated from all inlet channels, and which is used to supply a wash fluid to the plate settler or the collection channel of the bottom section so that the settled precipitated protein is drained (i.e., washed out) through the collection channel. This promotes the efficiency of a separation process. For example, when the precipitated protein tends not to be drained efficiently, possibly because there is a tendency to adhere to surfaces, such as parts of the bottom section, supplying a wash fluid may play an efficient contribution to collect the precipitated protein and to “wash” it down through a collection channel of the bottom section. A wash fluid may also promote the separation of the precipitated protein and the (remainder of) a supplied fluid. This may be of importance, because the fluid phase may still be of high value (e.g., it may contain further proteins of interest), and/or because it may contain impurities, which one wants to get rid of. Adjusting the composition and density of the wash fluid further improves the efficiency of the separation process.

The inventors have found that a plate settler resembling the one described above, which may be connected to a bottom section resembling the one described above, can also be used for continuously separating cells. Accordingly, in one embodiment the method for continuous recovering of a protein from a fluid in accordance with the present invention comprises the culturing of protein-producing cells in a fluid (e.g., a cell culture medium), such that the cells release the protein into the fluid, and the subsequent separation of the cells from the fluid using a plate settler for cell separation.

Finally, the inventors have unexpectedly found that the protein recovering of the present invention is particularly efficient when, after separating the protein precipitate from the rest fluid, the precipitated protein is re-solubilized using EDTA. Before the present invention, EDTA had been excluded as a potential candidate for re-solubilization, because its high complexing capability for calcium was assumed to be detrimental for protein (e.g., Factor VIII) activity.

In the above-described method for continuous recovering of a protein from a fluid, it is particularly advantageous when the plate settler comprises at least one sedimentation channel for letting the precipitated protein settle, which is relatively long. Accordingly, the present invention also provides a plate settler comprising a sedimentation channel with a length between 20 cm and 150 cm, preferably between 40 cm and 60 cm, most preferably about 50 cm.

Overall, the present invention provides an improved method for continuous recovering of a protein from a fluid, as well as an improved plate settler, by providing the preferred embodiments described below:
1. Method for continuous recovering of a protein from a fluid, wherein the method comprises the following steps:
   a protein precipitation step of precipitating the protein in the fluid; and
   a protein separation step of separating the precipitated protein from the fluid;
   wherein all steps are performed in an integrated process.
2. The method of item 1, wherein all steps of the method are performed continuously.
3. The method of item 1 or item 2, wherein the protein has a molecular weight of 250 kDa or more, preferably wherein the protein has a molecular weight of 500 kDa or more.
4. The method of any one of items 1 to 3, wherein before the protein precipitation step the concentration of the protein in the fluid comprising the protein is below 20 μg/ml, preferably between 0.05 μg/ml and 20 μg/ml.
5. The method of any one of items 1 to 4, wherein the protein is a blood coagulation factor.
6. The method of item 5, wherein the protein is Factor VIII.
7. The method of any one of items 1 to 4, wherein the protein is von Willebrand factor.
8. The method of any one of items 1 to 4, wherein the protein is a protein complex comprising Factor VIII and von Willebrand factor.
9. The method of any one of items 1 to 8, wherein in the protein precipitation step the protein is precipitated using a precipitant.
10. The method of item 9, wherein the precipitant is selected from the group consisting of calcium phosphate, polyethylene glycol (PEG), an affinity ligand, a pH modifying agent, an organic solvent such as ethanol or acetone, a polyelectrolyte such as polyacrylic acid or polyethylenimine, and a salt.
11. The method of item 9 or 10, wherein the precipitant comprises phosphate.
12. The method of any one of items 9 to 11, wherein the precipitant is calcium phosphate, magnesium phosphate, or zinc phosphate.
13. The method of any one of items 9 to 12, wherein the precipitant is calcium phosphate.
14. The method of item 13, wherein the protein precipitation step comprises adding calcium ions to the fluid.
15. The method of item 14, wherein calcium ions are added to a final concentration of between 10 mM and 50 mM, preferably between 10 mM and 30 mM.
16. The method of item 14, wherein calcium ions are added to a final concentration of between 10 mM and 20 mM, preferably about 15 mM.
17. The method of any one of items 13 to 16, wherein the protein precipitation step comprises adding phosphate ions to the fluid.
18. The method of item 17, wherein phosphate ions are added to a final concentration of between 1 mM and 10 mM, preferably between 1 mM and 5 mM.
19. The method of item 17, wherein phosphate ions are added to a final concentration of between 1 mM and 3 mM, preferably about 2 mM.
20. The method of any one of items 9 to 19, wherein the protein precipitation step comprises mixing the fluid comprising the protein and the precipitant.
21. The method of item 20, wherein mixing is performed in at least one reactor selected from the list consisting of a continuous stirred tank reactor (CSTR), a tubular reactor (TR), a segmented flow reactor, and an impinging jet reactor.
22. The method of item 20 or 21, wherein mixing is performed in a continuous stirred tank reactor (CSTR).
23. The method of any one of items 1 to 22, wherein the pH of the fluid before precipitating the protein is adjusted to a pH of between 8.5 and 9.0, preferably to a pH of about 8.75.
24. The method of any one of items 1 to 23, wherein the pH of the fluid after precipitating the protein is between 6 and 7.5, preferably between 6.5 and 7, most preferably about 6.5.
25. The method of any one of item 1 to 24, wherein in the protein separation step a plate settler for protein separation, continuous tangential flow filtration, or fluidized bed centrifugation is used for separating the precipitated protein from the fluid.
26. The method of any one of items 1 to 25, wherein in the protein separation step a plate settler for protein separation is used for separating the precipitated protein from the fluid.
27. The method of item 26, wherein the plate settler for protein separation is an inclined plate settler with a lower portion, an upper portion, and at least one sedimentation channel for letting the precipitated protein settle, said sedimentation channel extend from the lower portion to the upper portion,
   the inclined plate settler being configured to be oriented during use such that the at least one sedimentation channel extends from the lower portion to the upper portion in a direction that is inclined with respect to the direction of gravity,
   wherein the at least one sedimentation channel is connected to a fluid outlet for draining a rest fluid at the upper portion.
28. The method of item 27, wherein the length of the sedimentation channel is between 20 cm and 150 cm, preferably between 20 cm and 100 cm, more preferably between 20 cm and 80 cm, more preferably between 30 cm and 70 cm, more preferably between 40 cm and 60 cm, most preferably about 50 cm.
29. The method of item 27 or 28, wherein the at least one sedimentation channel of the plate settler for protein separation is connected to a bottom section, wherein the bottom section comprises at least one inlet channel for feeding the fluid comprising the precipitated protein to the plate settler, and at least one collection channel for collecting the settled precipitated protein descending from the at least one sedimentation channel,
   wherein said at least one inlet channel and said at least one collection channel are fluidly separated from each other, said inlet channel and said collection channel being connected to said at least one sedimentation channel, to form fluid connections between said at least one inlet channel and said at least one sedimentation channel and between said at least one collection channel and said at least one sedimentation channel, respectively.
30. The method of item 29, wherein the bottom section that is connected to the plate settler for protein separation further comprises at least one wash fluid supply channel for supplying a wash fluid to one sedimentation channel or to one collection channel, said at least one wash fluid supply channel being fluidly separated from other wash fluid supply channels and from all inlet channels.
31. The method of item 30, wherein the at least one wash fluid supply channel and the at least one collection channel corresponding to the same sedimentation channel of the plate settler for protein separation are fluidly connected by an opening in a wall portion shared by said wash fluid supply channel and said collection channel.
32. The method of item 30 or item 31, wherein the fluid comprising the precipitated protein is supplied to the bottom section, which is connected to the plate settler for protein separation, through the at least one inlet channel, and a wash fluid is supplied through the at least one wash fluid supply channel,
   wherein the density of the wash fluid is higher than the density of the fluid comprising the precipitated protein, and
   wherein the rest fluid is drained through the fluid outlet at the upper portion and the settled precipitated protein is drained through the collection channel.
33. The method of item 32, wherein the density of the wash fluid is between 0.3% and 1.5% higher than the density of the fluid comprising the precipitated protein, preferably between 0.55% and 1.20% higher than the density of the fluid comprising the precipitated protein.
34. The method of item 32 or 33, wherein the wash fluid comprises Tris and sodium chloride.
35. The method of item 34, wherein the wash fluid comprises Tris at a concentration of about 2 mM and sodium chloride at a concentration of about 272 mM.
36. The method of item 34 or 35, wherein the wash fluid further comprises calcium chloride.
37. The method of item 36, wherein the wash fluid comprises calcium chloride at a concentration of between 4 mM and 12 mM.
38. The method of item 36 or 37, wherein the wash fluid comprises Tris at a concentration of about 2 mM, sodium chloride at a concentration of about 231 mM and calcium chloride at a concentration of about 12 mM.
39. The method of any one of items 32 to 38, wherein the wash fluid has a pH of 7.5 or higher, preferably of 8 or higher, most preferably of about 8.25.
40. The method of any one of items 32 to 39, wherein the wash fluid is supplied through the at least one wash fluid supply channel and the settled precipitated protein is drained through the collection channel at regular intervals.
41. The method of item 40, wherein the wash fluid is supplied through the at least one wash fluid supply channel and the settled precipitated protein is drained through the collection channel at regular intervals of between 15 min and 45 min, preferably about 30 min.
42. The method of any one of items 32 to 41, wherein the wash fluid is supplied through the at least one wash fluid supply channel and the settled precipitated protein is drained through the collection channel at a volumetric flow rate of about 20 to 60 mL/min, preferably about 40 mL/min.
43. The method of any one of items 1 to 42, wherein the method further comprises the following steps before the protein precipitation step:
   a protein production step of culturing cells in a fluid, wherein the cells produce the protein and release the protein into the fluid; and
   a cell separation step of separating the cells from the fluid comprising the protein.
44. The method of item 43, wherein the cells are mammalian cells.
45. The method of item 44, wherein the cells are CHO cells.
46. The method of any one of items 43 to 45, wherein the fluid is a cell culture medium.
47. The method of any one of items 43 to 46, wherein in the protein production step the cells are cultured in a perfusion reactor or a chemostat reactor, preferably in a chemostat reactor.
48. The method of any one of items 43 to 47, wherein in the cell separation step a plate settler for cell separation is used for separating the cells from the fluid comprising the protein.
49. The method of item 48, wherein the plate settler for cell separation is an inclined plate settler with a lower portion, an upper portion, and at least one sedimentation channel for letting the cells settle, said sedimentation channel extend from the lower portion to the upper portion,
   the inclined plate settler being configured to be oriented during use such that the at least one sedimentation channel extends from the lower portion to the upper portion in a direction that is inclined with respect to the direction of gravity,
   wherein the at least one sedimentation channel is connected to a fluid outlet for draining a rest fluid at the upper portion.
50. The method of item 49, wherein the at least one sedimentation channel of the plate settler for cell separation is connected to a bottom section, wherein the bottom section comprises at least one inlet channel for feeding the fluid comprising the cells and the protein to the plate settler, and at least one collection channel for collecting the settled cells descending from the at least one sedimentation channel,
   wherein said at least one inlet channel and said at least one collection channel are fluidly separated from each other, said inlet channel and said collection channel being connected to said at least one sedimentation channel, to form fluid connections between said at least one inlet channel and said at least one sedimentation channel and between said at least one collection channel and said at least one sedimentation channel, respectively.
51. The method of item 50, wherein the bottom section that is connected to the plate settler for cell separation further comprises at least one wash fluid supply channel for supplying a wash fluid to one sedimentation channel or to one collection channel, said at least one wash fluid supply channel being fluidly separated from other wash fluid supply channels and from all inlet channels.
52. The method of item 51, wherein the at least one wash fluid supply channel and the at least one collection channel corresponding to the same sedimentation channel of the plate settler for cell separation are fluidly connected by an opening in a wall portion shared by said wash fluid supply channel and said collection channel.
53. The method of item 51 or 52, wherein the fluid comprising the cells and the protein is supplied to the bottom section, which is connected to the plate settler for cell separation, through the at least one inlet channel, and a wash fluid is supplied through the at least one wash fluid supply channel,
   wherein the density of the wash fluid is higher than the density of the fluid comprising the cells and the protein, and
   wherein the settled cells are drained through the collection channel and the rest fluid comprising the protein is drained through the fluid outlet at the upper portion.
54. The method of item 53, wherein the wash fluid is supplied through the at least one wash fluid supply channel and the settled cells are drained through the collection channel at regular intervals.
55. The method of item 53 or 54, wherein the wash fluid is supplied through the at least one wash fluid supply channel and the settled cells are drained through the collection channel at regular intervals of 5 min to 90 min.
56. The method of item 53 or 54, wherein the wash fluid is supplied through the at least one wash fluid supply channel and the settled cells are drained through the collection channel at regular intervals of 15 min to 85 min.
57. The method of item 53 or 54, wherein the wash fluid is supplied through the at least one wash fluid supply channel and the settled cells are drained through the collection channel at regular intervals of 25 min to 80 min.
58. The method of item 53 or 54, wherein the wash fluid is supplied through the at least one wash fluid supply channel and the settled cells are drained through the collection channel at regular intervals of 35 min to 75 min.
59. The method of item 53 or 54, wherein the wash fluid is supplied through the at least one wash fluid supply channel and the settled cells are drained through the collection channel at regular intervals of 45 min to 70 min.
60. The method of item 53 or 54, wherein the wash fluid is supplied through the at least one wash fluid supply channel and the settled cells are drained through the collection channel at regular intervals of 55 min to 65 min.
61. The method of item 53 or 54, wherein the wash fluid is supplied through the at least one wash fluid supply channel and the settled cells are drained through the collection channel at regular intervals of about 60 min.
62. The method of any one of items 53 to 61, wherein the wash fluid is supplied through the at least one wash fluid supply channel and the settled cells are drained through the collection channel at a volumetric flow rate of between 50 to 70 mL/min, preferably about 60 mL/min.
63. The method of any one of items 1 to 62, wherein the method further comprises the following step after the protein separation step:
   a re-solubilization step of re-solubilizing the precipitated protein.
64. The method of item 63, wherein in the re-solubilization step the precipitated protein is re-solubilized using citrate or EDTA.
65. The method of item 64, wherein in the re-solubilization step the precipitated protein is re-solubilized using EDTA.
66. The method of item 65, wherein in the re-solubilization step the precipitated protein is re-solubilized using EDTA at a final concentration of between 10 mM to 50 mM.
67. The method of item 65 or 66, wherein in the re-solubilization step the precipitated protein is re-solubilized using EDTA at a final concentration of between 20 mM to 30 mM.
68. The method of any one of items 65 to 67, wherein in the re-solubilization step the precipitated protein is re-solubilized using EDTA at a final concentration of about 25 mM.
69. The method of any one of items 1 to 68, wherein the protein is a biopharmaceutical drug.
70. Recovered protein that is obtainable by the method of any one of items 1 to 69.
71. Method for producing a pharmaceutical composition, comprising performing the method of item 69 and formulating the recovered biopharmaceutical drug as a pharmaceutical composition.
72. Pharmaceutical composition that is obtainable by the method of item 71.
73. An inclined plate settler for separating a solid component from a fluid, wherein the plate settler comprises a lower portion, an upper portion, and at least one sedimentation channel for letting the solid component settle, said sedimentation channel extend from the lower portion to the upper portion,
   the plate settler being configured to be oriented during use such that the at least one sedimentation channel extends from the lower portion to the upper portion in a direction that is inclined with respect to the direction of gravity,
   wherein the at least one sedimentation channel is connected to a fluid outlet for draining a rest fluid at the upper portion and connected to a bottom section at the lower portion,
   wherein the bottom section comprises at least one inlet channel for feeding a fluid comprising the solid component to be separated to the plate settler, and at least one collection channel for collecting a settled component descending from the at least one sedimentation channel,
   wherein said at least one inlet channel and said at least one collection channel are fluidly separated from each other, said inlet channel and said collection channel being connected to said at least one sedimentation channel, to form fluid connections between said at least one inlet channel and said at least one sedimentation channel and between said at least one collection channel and said at least one sedimentation channel, respectively.
   wherein the bottom section further comprises at least one wash fluid supply channel for supplying a wash fluid to one sedimentation channel or to one collection channel, said at least one wash fluid supply channel being fluidly separated from other wash fluid supply channels and from all inlet channels, and
   wherein the length of the sedimentation channel is between 20 cm and 150 cm, preferably between 20 cm and 100 cm, more preferably between 20 cm and 80 cm, and most preferably between 30 cm and 70 cm.
74. The inclined plate settler of item 73, wherein the length of the sedimentation channel is between 40 cm and 60 cm, preferably about 50 cm.
75. The inclined plate settler of item 73 or 74, wherein the solid component is a precipitated protein, preferably a precipitated protein complex comprising Factor VIII and von Willebrand factor.
76. The inclined plate settler of any one of items 73 to 75, wherein the inclined plate settler contains a precipitated protein complex comprising Factor VIII and von Willebrand factor.

### BRIEF DESCRIPTION OF THE DRAWINGS

FIG. 1 is a sectional view of a schematic representation of an embodiment of a bottom section in accordance with the present disclosure;

FIG. 2 is a sectional view of a schematic representation of an embodiment of a bottom section in accordance with the present disclosure;

FIG. 3 is a schematic three dimensional perspective view of an embodiment of a bottom section and, more generally, of an assembly with a plate settler in accordance with the present disclosure;

FIG. 4 is a sectional view of an inlet channel, a collection channel, and a wash fluid supply channel of an embodiment of a bottom section in accordance with the present disclosure;

FIG. 5 is a schematic three dimensional perspective view of an embodiment of a bottom section in accordance with the present disclosure;

FIG. 6 is a schematic three dimensional perspective view of an embodiment of a bottom section in accordance with the present disclosure;

FIG. 7 is a schematic representation of a flow distributor which forms part of an embodiment of a bottom section in accordance with the present disclosure;

FIG. 8A is a schematic representation of a flow distributor that is part of an embodiment of a bottom section in accordance with the present disclosure;

FIG. 8B is a schematic representation of a flow distributor that is part of an embodiment of a bottom section in accordance with the present disclosure;

FIG. 8C is a schematic representation of a flow distributor that is part of an embodiment of a bottom section in accordance with the present disclosure;

FIG. 9A is a schematic representation of a split in a flow distributor that is part of an embodiment of a bottom section in accordance with the present disclosure;

FIG. 9B is a schematic representation of a split in a flow distributor that is part of an embodiment of a bottom section in accordance with the present disclosure;

FIG. 9C is a schematic representation of a split in a flow distributor that is part of an embodiment of a bottom section in accordance with the present disclosure;

FIG. 9D is a schematic representation of a split in a flow distributor that is part of an embodiment of a bottom section in accordance with the present disclosure;

FIG. 9E is a schematic representation of a split in a flow distributor that is part of an embodiment of a bottom section in accordance with the present disclosure;

FIG. 9F is a schematic representation of a split in a flow distributor that is part of an embodiment of a bottom section in accordance with the present disclosure;

FIG. 10 is a schematic representation an embodiment of a bottom section and, more generally, of an assembly with a plate settler in accordance with the present disclosure;

FIG. 11 is a schematic representation an embodiment of a bottom section in accordance with the present disclosure;

FIG. 12 is a schematic representation an embodiment of a bottom section in accordance with the present disclosure; and

FIG. 13 is a schematic representation an embodiment of a bottom section and, more generally, of an assembly with a plate settler in accordance with the present disclosure.

FIG. 14 Schematic drawing of the assembly of bioreactor [1] and inclined plate settler in assembly with the bottom section [3] as used in example 1. The assembly included multiple pumps [2] via which the cell culture broth was transported to the assembly, the wash solution [5] was supplied to the bottom section and the solids (cells) [6] were collected from the bottom section. The clarified fluid was collected at the top outlet of the assembly [4]. The dashed lines indicate the double jacket and the cryostat, which make up an additional fluid circuit [7] that was not fluidly connected to the cell culture broth, the solid depleted fluid or the collected solids (cells).

FIG. 15 Product (FVIII) yield and recovery in the fluid streams collected from the top and bottom outlets of the inclined plate settler in assembly with the bottom section under temperature control via double jacket as described by example 1. Recovery=sum of yield in both streams leaving the inclined plate settler and bottom section assembly. The top and bottom panels show the results of two separate runs.

FIG. 16 Glucose yield and recovery in the fluid streams collected from the top and bottom outlets of the inclined plate settler in assembly with the bottom section under temperature control via double jacket as described in example 1. Recovery=sum of yield in both streams leaving the inclined plate settler and bottom section assembly. The top and bottom panels show the results of two separate runs.

FIG. 17 Schematic drawing of the assembly of bioreactor [1] and inclined plate settler in assembly with bottom section [3] as used in example 2. The assembly included multiple pumps [2] via which cell culture broth was transported to the assembly, the wash solution [5] was supplied to the bottom section and the solids [6] were collected from the bottom section. The clarified fluid was collected at the top outlet of the assembly [4]. The entire setup with exception of the bioreactor was situated in a cold room at 2-8° C.

FIG. 18 Product yield and recovery (top) and glucose yield and recovery (bottom) in the fluid streams collected from the top and bottom outlet of the inclined plate settler and the bottom section as described by example 2 (corresponding FIG. 17). Recovery=sum of yield in both streams leaving the inclined plate settler and bottom section assembly.

FIG. 19 Product yield and recovery (top) and glucose yield and recovery (bottom) in the fluid streams collected from the top and bottom outlet of the inclined plate settler and the bottom section as described in example 3 (corresponding FIG. 17). Recovery=sum of yield in both streams leaving the inclined plate settler and bottom section assembly.

FIG. 20 Schematic drawing of the bottom section in assembly with the inclined plate settler [5] connected to a supplying vessel [1], which could be, a bioreactor or a vessel containing a process fluid such as 1 M sodium hydroxide or buffer. The assembly comprises three-way-valves for switching between different fluid paths (marked with *) and three-way-valves for sampling (marked with +). Further, it comprises a vessel for supply of a wash solution [2], a receiving vessel for, e.g. an exhaust fluid [3], a receiving vessel for the collected solids [4] and a receiving vessel for solid depleted fluid [6]. All receiving vessels comprise an additional connection that encompasses a sterile filter, thus pressure exchange is possible without compromising the aseptic conditions within the assembly.

FIG. 21 Yield of Tryptophan in the fraction containing the collected solids (i.e. the precipitate) suspended in wash fluid obtained at varying collection flow rates. Tryptophan was originally comprised in the precipitate suspension.

FIG. 22 Yield of Patent Blue V in the fraction containing the collected solids suspended in wash fluid obtained at varying collection flow rates. Patent Blue V was originally comprised in the wash fluid.

FIG. 23 Custom built settling monitoring device: Measuring cylinder equipped with photo emitter and detector for turbidity measurement during settling of precipitate.

FIG. 24A-B Results of calcium, phosphate and citrate concentrations for precipitation of the FVIII:VWF complex and dissolution of the same. The sample code translates as Ca conc. [mM]/PO₄ conc. [mM]/Citrate:Ca. A—without pH modification. B—pH modification with TRIS as buffering agent. Left y-axis=Yield of FVIII and VWF. Right y-axis: Volume reduction factor.

FIG. 25 Precipitation of FVIII:VWF complex from CCSN by varying calcium and phosphate concentrations. Calcium concentration as indicated in the top left corner of each set of results. Phosphate concentration left to right=1.5, 2.0 and 2.5 mM. All samples pH modified using TRIS buffer. Error bars represent RSD for a set of five physical replicates.

FIG. 26 Calcium dependent precipitation behavior of VWF observed in precipitation of the FVIII:VWF complex by calcium phosphate. Precipitation at constant phosphate conc. (2 mM) and pH modification using TRIS buffer. Error bars represent three physical replicates.

FIG. 27 Yield of FVIII and VWF after precipitation of FVIII:VWF from CCSN at different starting pH values using 15 mM CaCl₂ and 2 mM phosphate. pH modification with 0.1 M HCl and 0.1. M NaOH as needed. Error bars correspond to three physical replicates.

FIG. 28 SDS-PAGE of calcium phosphate precipitated cell culture supernatant samples (15 mM Ca²⁺, 2 mM PO₄, pH 8.5 prior to precipitation). 1—HiMark™ pre-stained standard. 2—VWF BDS. 3—FVIII BDS. 4—clarified cell culture supernatant. 5—precipitation supernatant. 6—dissolved calcium phosphate precipitate undiluted. 7—dissolved calcium phosphate precipitate diluted 1:2.

FIGS. 29A-B FVIII (A) and VWF (B) precipitation supernatant concentration obtained in precipitation kinetic studies under buffered and unbuffered conditions (pH modification with 2 M TRIS and 1 M NaOH, respectively).

FIG. 30 Yield of FVIII and VWF in adsorption and elution experiments with different kinds of calcium phosphate (solid phases) in the CCSN after incubation with calcium phosphate and the corresponding elution or dissolution fractions. A—in situ formed calcium phosphate. B—ex situ formed (wet) calcium phosphate added to CCSN. C—CHT I resin. D—CHT II resin.

FIG. 31A-B Residence time distribution curves for single phase (H₂O and 1 M NaCl) and two phase (calcium phosphate precipitate) tracer experiments. (A) Shows the entire data set and (B) shows a zoomed in version of the same plot.

FIG. 32A-B (A) Normalized turbidity signals obtained during sedimentation of calcium phosphate precipitate (50 mM TRIS, 15 mM calcium, 2 mM phosphate) in a custom-built sedimentation-monitoring device. (B) Final turbidity level obtained after ˜30 min sedimentation time of calcium phosphate. Error bars represent standard deviation of physical replicates.

FIG. 33 Maximum settling velocity of calcium phosphate produced in batch and continuous precipitation using different reactor configurations. Error bars correspond to the standard deviation of physical replicates.

FIG. 34A-B Yield of VWF (A) and FVIII (B) obtained in precipitation experiments performed in batch (in triplicate) or in continuous mode. Continuous precipitation was performed with three different reactor configurations: CSTR, TR+CSTR and TR. CCSN was adjusted to pH 9.0 and supplemented with 2 mM phosphate. Precipitation was initiated by addition of 15 mM CaCl₂.

FIG. 35 Schematic drawing of the prototype setup for continuous precipitation and precipitate collection using the inclined plate settler. Pumps are labelled with P and their respective numbers. Temp-I=temperature indicator. pH-C=pH control loop. Level-I=level indicator for fill level control of stirred vessels. TR=tubular reactor. pH-I=pH indicator without control function. S=sampling valves with corresponding numbers. B-T=bubble trap. T-I=turbidity indicator.

FIG. 36A-C Results from experiment 1-01. (A) Overlay of pH value in CSTR with FVIII and VWF yield in DP. (B) VWF yield (C) FVIII yield. Yield values are plotted for surge tank (ST), supernatant (SN), dissolved precipitate (DP) and recovery (Rec)=SN+DP. 1 system volume ˜150 mL.

FIG. 37A-C Results from experiment 1-02. (A) Overlay of pH value in CSTR with FVIII and VWF yield in DP. (B) VWF yield (C) FVIII yield. Yield values are plotted for surge tank (ST), supernatant (SN), dissolved precipitate (DP) and recovery (Rec)=SN+DP. 1 system volume ˜150 mL.

FIG. 38A-C Results from experiment 1-03. (A) Overlay of pH value in CSTR with FVIII and VWF yield in DP. (B) VWF yield (C) FVIII yield. Yield values are plotted for surge tank (ST), supernatant (SN), dissolved precipitate (DP) and recovery (Rec)=SN+DP. 1 system volume ˜150 mL.

FIG. 39A-C Results from experiment 1a-01 (without tubular reactor). (A) Overlay of pH value in CSTR with FVIII and VWF yield in DP. (B) VWF yield (C) FVIII yield. Yield values are plotted for surge tank (ST), supernatant (SN), dissolved precipitate (DP) and recovery (Rec)=SN+DP. 1 system volume ˜150 mL.

FIG. 40A-C Results from experiment 2-01 (without tubular reactor). (A) Overlay of pH value in CSTR with FVIII and VWF yield in DP. (B) VWF yield (C) FVIII yield. Yield values are plotted for surge tank (ST), supernatant (SN), dissolved precipitate (DP) and recovery (Rec)=SN+DP. 1.

FIG. 41A-B Batch reference precipitation of FVIII:VWF from fresh CCSN using calcium phosphate. pH modification=sample taken after pH modification in batch. DP=dissolved precipitate. SN=precipitation supernatant. (A) VWF yield; (B) FVIII yield.

FIG. 42A-B Pictures from settling experiments to check for suitability of buffer density for use in an inclined plate settler. CCSN precipitated with 15 mM CaCl₂ and 2 mM phosphate Buffer composition. (A) 2 mM TRIS, pH 8.25 with 100 mg/L Patent Blue V, 231 mM NaCl and 12 mM CaCl₂. (B) 2 mM TRIS, pH 8.25 with 100 mg/L Patent Blue V, 272 mM NaCl.

FIG. 43 Trade-off between CaCl₂ supplementation and NaCl concentration required for equal density of wash buffers to be used in the inclined plate settler.

FIG. 44A-B Wash buffer influence on product yield with precipitate settling into the wash buffer in a separation funnel with the 100% reference being (A) CCSN (=starting material) and (B) the precipitated reference sample that was not washed. Error bars represent standard deviation of three physical replicates obtained on two different days.

FIG. 45A-B Yield of tracers in the discharged fraction at different flow rates applied during the discharge interval. Patent Blue V was supplemented to the wash buffer (A). Tryptophan was added to the feed stream (B).

FIG. 46A-C Overlay of discharge peaks obtained at different discharge flow rates: (A) 20 mL/min. (B) 40 mL/min. (C) 60 mL/min.

FIG. 47A-B Yield based on tracer measurements (A) in the top overflow (B) and in the discharge fractions at discharge intervals between 30 and 60 min.

FIG. 48A-C Overlay of discharge peaks obtained at 40 mL/min discharge flow rate and different discharge intervals: (A) 30 min. (B) 45 min. (C) 60 min.

FIGS. 49A-B (A) Patent Blue V Yield and (B) Patent Blue V relative concentration in the discharged fractions at different discharge volumes. Discharge flow=40 mL/min, discharge interval=30 min. Max PBV concentration: (12.8 mL)=69%, (22.8 mL)=82%, (45 mL)=89%.

FIG. 50A-C Overlay of discharge peaks obtained at 40 mL/min discharge flow rate, 30 min discharge interval and discharge volumes of (A) 45 mL (data from previous experiments, for comparison). (B) 22.8 mL. (C) 12.8 mL.

FIG. 51 Data recorded during the integration of the continuous precipitation with the inclined plate settler.

Left y-axis: Feed turbidity as recorded in the settler software. Right y-axis: pH in the surge tank and CSTR as recorded in the precipitation software.

FIG. 52A-B Yield of VWF (A) and FVIII (B) obtained by continuous precipitation integrated with continuous solid-liquid separation (i.e. inclined plate settler). ST=surge tank. CSTR=sample after precipitation, before settler. TOP=settler overflow. DP=settler discharge fraction dissolved precipitate. WB=wash buffer, settler discharge fraction liquid phase. Rec=recovery, DP+WB+TOP.

FIG. 53 Turbidity signals recorded in the plate settler software during the integration run with the continuous precipitation setup. From top to bottom: feed, top and sludge turbidity. The vertical lines represent the sampling points.

FIG. 54A-B Results of pH stability tests for FVIII and VWF in complex (A and B, respectively). Error bars represent the RSD of three analytical replicates. Where no error bars are visible, the samples were quantified only once.

FIG. 55A-B Results of pH stability tests for FVIII and VWF after split of the complex (A and B, respectively).

Error bars represent the RSD of three analytical replicates. Where no error bars are visible, the samples were quantified only once.

FIG. 56A-B Cell removal performance using an inclined settler with a structured bottom section at a starting cell density of 1.5×10\hat{ }6 cells/mL. Average starting turbidity 46.2 NFU. (A) Clarification efficiency based on relative reduction of and absolute values for cell count and turbidity. (B) Separation efficiency based on Glucose as a surrogate for product. The dashed line indicates 5% yield.

FIG. 57 Discharge peaks obtained during a run at 1.5×10\hat{ }6 cells/mL. The run was performed using the structured bottom section and the acrylic glass settling section. The line color changes with the No. of discharge cycle over time from black to grey.

FIG. 58 Product yield obtained in cell removal using a structured bottom section in combination with an acrylic glass settling section. The starting cell density was 1.5×10\hat{ }6 cells/mL. The dashed line indicates 5% yield (left y-axis).

FIG. 59A-B Cell removal performance using an inclined settler with a conventional, open bottom section. Starting cell density was of 1.5×10\hat{ }6 cells/mL. Average starting turbidity 57.6 NFU. (A) Clarification efficiency based on cell count and turbidity with relative and absolute values. (B) Separation efficiency based on Glucose as a surrogate for product.

FIG. 60A-B (A) Complementary FVIII activity-based yield obtained during cell removal with a conventional, open bottom section. (B) Discharge peaks obtained by collection of removed cells from the conventional bottom section. Color gradient over time with early discharge peaks in black and late discharge peaks in grey.

FIG. 61 FVIII yield in dissolved precipitate samples obtained on two different days with analysis on the same day, resulting in an incubation of ˜24 h for day 1 samples.

FIG. 62 FVIII yield in dissolved precipitate samples obtained on four different days with centrifugation at 4800 rcf on the first and 1000 rcf on the following three days. FVIII analytics were performed directly after re-solubilization of the corresponding samples.

FIG. 63 VWF yield in dissolved precipitate samples obtained on three different days by centrifugation at 1000 rcf. These results correspond to the 2nd to 4th day of FIG. 62. VWF analytics for all samples were performed on one day using aliquots stored at <−60° C.

FIG. 64A-B Batch precipitation of fresh cell culture supernatant with increasing pH prior to precipitation. (A) FVIII yield determined directly after re-solubilization. (B) VWF yield determined from thawed samples.

FIG. 65 Results of replicate batch precipitation experiments performed with fresh clarified harvest, where the pH was set to 8.5 or 8.75 prior precipitation. Error bars represent three re-solubilized aliquots from one precipitation event.

FIG. 66A-D Overlay of FVIII yield in the Surge tank at pH 8.75 (after pH modification), in the precipitation supernatant and in the dissolved precipitate with the observed pH in the CSTR (i.e. in the precipitate suspension) for four different precipitation experiments performed on four different days (A, B, C and D, respectively).

FIG. 67A-B Average yield in the precipitation supernatant and the dissolved precipitate obtained in the continuous precipitation experiments with experiments labelled by date. A—FVIII results. B—VWF results. Error bars represent standard deviation of 5 samples taken during the course of the individual experiments.

### DETAILED DESCRIPTION OF THE INVENTION

All publications, patents and patent applications cited herein are hereby incorporated by reference in their entireties for all purposes.

### Definitions

Unless otherwise defined below, the terms used in the present invention shall be understood in accordance with their common meaning known to the person skilled in the art.

The term “continuous” as used herein refers to processes that are capable of being operated (e.g., at steady state) with a continuous (i.e., uninterrupted) inflow, and produce a continuous (i.e., uninterrupted) or a semi-continuous (i.e., discretized) output. The point of stable operation, or steady state, can, but does not have to be, the system's equilibrium.

Accordingly, a “method for continuous recovering of a protein from a fluid” as used herein refers to a protein recovering method which is capable of being operated such that the process as a whole has a continuous inflow (e.g., of the fluid comprising the protein in accordance with the present invention), and then produces a continuous or semi-continuous output (e.g., of fluid and (recovered) protein).

The term “integrated process” as used herein refers to a process that is operated using an apparatus wherein all (sub-)units are physically connected. This apparatus can be, but does not have to be, a modular apparatus. Thus, a “method for continuous recovering of a protein from a fluid” wherein “all steps are performed in an integrated process” refers to method which is capable of being operated with continuous inflow of fluid, and a continuous or semi-continuous output of fluid and protein, wherein all units are physically connected, such that the fluid that is supplied to the apparatus is led through all units of the apparatus without physically leaving the apparatus before the fluid as well as the protein are drained as output.

The term “fluid” as used herein is used synonymously with the term “liquid” and refers to any matter in the liquid state. The “fluid” or “liquid” in accordance with the invention may also be a suspension, e.g. suspension that comprises cells and/or precipitate.

The term “recovering” as used herein refers to any process that separates a substance of interest from other substances, and thereby removes these other substances from the substance of interest. The removal does not have to be a complete removal, i.e. residual amounts of the other substances may still remain with the substance of interest. Accordingly, the term “recovering of a protein from a fluid” as used herein refers to the separation and removal of the fluid (as well as at least some of the components that may be contained, e.g., dissolved, in the fluid) from the protein, although the separation and removal does not have to be complete. Typically, such recovering leads to a volume reduction, and thus to a concentration of the protein of interest.

Consistent with the meaning of the term “recovering” as used herein, the term “separation” or “separate” as used herein does not imply that two or more substances are completely separated. Thus, the term “separation” or “separate” can also be used for a process wherein two or more substances are separated such that residual amounts of the one substance remain with the other substance, and vice versa.

The term “precipitation” as used herein refers to a process wherein a substance that is dissolved in a fluid becomes part of a solid phase. This may mean that the substance itself changes its state of aggregation and becomes solid, and/or that the substance remains dissolved but, following precipitation, is present within the solid phase. For example, during precipitation of a protein using calcium phosphate, the majority of the formed solid may be calcium phosphate. Only a small fraction of the formed solid may be protein. However, much of the protein that may remain dissolved in the fluid may be present in the fluid that is present in the interstitial space between and within the flocs of solid calcium phosphate. Such dissolved protein in the interstitial space between and within the flocs of solid calcium phosphate is also referred to as “precipitated protein” in the present invention.

The term “plate settler” as used herein has the meaning known to the skilled person. Preferably, the “plate settler” in accordance with the present invention is an “inclined plate settler”. Examples of inclined plate settlers are disclosed in US 2012/0302741 A1, U.S. Pat. Nos. 2,793,186 A1, 753,646 A1, and US 2002/0074265 A1. In accordance with the present invention, a plate settler can be used to separate precipitated protein from a fluid, in which case the plate settler is also referred to as “plate settler for protein separation”. However, in accordance with the present invention a plate settler can also be used to separate cells from a fluid, in which case the plate settler is also referred to as “plate settler for cell separation”. Many optional embodiments of the “plate settler for protein separation” and the “plate settler for cell separation” as well as of the bottom sections that are preferably connected to these plate settlers in accordance with the present invention are identical, and in one embodiment of the present invention the “plate settler for protein separation” and the “plate settler for cell separation” as well as the bottom sections that may be connected to them are identical. However, in another embodiment in accordance with the present invention the “plate settler for protein separation” and the “plate settler for cell separation” as well as the bottom sections that may be connected to them differ in one or more features.

The term “final concentration” of a substance as used herein refers to the concentration of the substance in the (e.g. fluid) composition that is the direct result of adding said substance to said (e.g. fluid) composition. Thus, the “final concentration” does not include any amounts of a substance that may already be present in the (e.g. fluid) composition before adding said substance. For example, when calcium ions are added to a “final concentration” of, e.g., 15 mM, this means that calcium ions are added in such an amount that directly results in a concentration of 15 mM in the (e.g. fluid) composition. In this example, in order to add calcium ions to a final concentration of 15 mM, 1 L of calcium ions at a concentration of 1.5 M could be added to 99 L of a fluid composition, regardless of whether or not calcium ions had already been present in the fluid composition.

The term “descending” as used herein has the meaning known to the person skilled in the art. For example, the plate settler in accordance with the present invention may comprise at least one collection channel for collecting a settled solid component descending from the at least one sedimentation channel. As will be clear to a person skilled in the art, the term “descending” as used in this context refers to solid components that have already settled, i.e. that may already have descended from the at least one sedimentation channel.

In accordance with the present invention, each occurrence of the term “comprising” may optionally be substituted with the term “consisting of”.

### EMBODIMENTS

In the following specific embodiments of the invention will be described, but the invention is not limited thereto. Moreover, any of the below embodiments can be combined with any of the other below embodiments according to the present invention.

The method for continuous recovering of a protein from a fluid in accordance with the present invention comprises a protein precipitation step of precipitating the protein in the fluid; and a protein separation step of separating the precipitated protein from the fluid; wherein all steps are performed in an integrated process. This method is capable of being operated with a continuous (i.e., uninterrupted) inflow, and then produces a continuous (i.e., uninterrupted) or a semi-continuous (i.e., discretized) output. The output of this method is the (recovered) protein as well as the (residual) fluid from which the protein has been recovered (i.e., separated).

In a preferred embodiment, each step of the method for continuous recovering of a protein from a fluid in accordance with the present invention is performed continuously. In this embodiment, each step of the method is capable of being operated with a continuous (i.e., uninterrupted) inflow, and then produces a continuous (i.e., uninterrupted) or a semi-continuous (i.e., discretized) output.

All steps of the method for continuous recovering of a protein from a fluid in accordance with the present invention are performed in an integrated process. An integrated process is a process wherein all units within the apparatus that is used for the process are physically connected. Generally, the different steps of the method for continuous recovering of a protein from a fluid in accordance with the present invention have different requirements regarding the physical environment they are performed in. Thus, in one embodiment, each step of the method for continuous recovering of a protein from a fluid in accordance with the present invention is performed in at least one separate unit of the apparatus that is used for the method. In any case, all units within the apparatus that is used for the method for continuous recovering of a protein from a fluid in accordance with the present invention are physically connected.

In one embodiment, the protein precipitation step and/or the protein separation step of the method for continuous recovering of a protein from a fluid in accordance with the present invention is/are performed at a temperature of between 0° C. and 8° C., preferably of between 2° C. and 8° C. In a preferred embodiment, both the protein precipitation step and the protein separation step are performed at a temperature of between 0° C. and 8° C., preferably of between 2° C. and 8° C.

The method for continuous recovering of a protein from a fluid in accordance with the present invention comprises a protein precipitation step of precipitating the protein in the fluid. The method of precipitation is not particularly limited and includes, for example, precipitating the protein by heating the fluid comprising the protein, or precipitating the protein by adding a precipitant.

In a preferred embodiment, the protein in the fluid in accordance with the present invention is precipitated using a precipitant. In this case, the difference in solubility of two or more solutes is exploited. By addition of a precipitant, the solubility of a given solute is altered. Upon this change in solubility a solid phase, i.e. the precipitate, is formed. By precipitating the protein, a significant volume reduction can be achieved. Using protein precipitation in the method for recovering of a protein from a fluid in accordance with the present invention offers several advantages: Precipitation can be scaled up linearly, does not require complex equipment and can be performed under non-denaturizing conditions. Precipitants that can be used in accordance with the present invention include calcium phosphate, polyethylene glycol (PEG; preferably PEG with a molecular weight of 6,000 kDa or higher), an affinity ligand, a pH modifying agent, an organic solvent such as ethanol or acetone, a polyelectrolyte such as polyacrylic acid or polyethylenimine, and a salt.

Precipitation processes can be divided in different categories depending on the composition of the precipitate and the precipitant used. For the precipitate composition, two situations can be distinguished: In the first case, the precipitate consists almost entirely of the target molecule, as is the case e.g. in PEG precipitation. The second possibility is co-precipitation, in which the precipitate is a mixture of a solid and the target molecule captured by that solid, as appears to be the case for calcium phosphate precipitation. In the following, some precipitants and their mode of action are briefly explained, without wishing to be bound by theory. In affinity precipitation, the interaction between an affinity ligand and the target molecule is exploited. Affinity precipitation provides high specificity based on the high affinity binding between ligand and target. The affinity ligands provide crosslinking between the target molecules. With increasing size, the ligand-target complex becomes less soluble and is precipitated from the process solution. Changing the solution pH can directly be used to precipitate proteins, which is exploited in isoelectric precipitation. When the solution's pH equals the isoelectric point of a specific protein, the protein solubility is significantly reduced and precipitates can be formed. Proteins can be precipitated by addition of organic solvents to a process fluid, which causes a reduction in water activity and in the dielectric constant of the medium. Therefore, the solubility of charged, hydrophilic proteins is reduced up until the point of protein precipitation. Acetone and ethanol were reported to be the most prevalent solvents. Especially ethanol precipitation of proteins has extensively used at large scale in the process of blood plasma fractionation. Polyethylenglycol (PEG), which is a non-ionic polymer, was reported to behave similar to organic solvents. PEG is the most abundantly used polymer and is available in a broad range of molecular weights. For PEG, a linear correlation between log S and PEG concentration was found. The protein solubility, S, can be calculated according to Equation 1, where S₀ is the apparent intrinsic solubility obtained by extrapolation to zero PEG, β is the slope and C is the PEG concentration:

log S=log S₀−βC (Equation 1)

The precipitation of proteins using PEG is largely independent of solution pH, ionic strength and temperature, which makes for a robust process. However, removing PEG from the protein-containing fraction after precipitation can be challenging. Besides non-ionic polymers, polyelectrolytes can be used for precipitation. Examples for polyelectrolytes are polyacrylic acid and polyethylenimine. Finally, proteins can be precipitated by addition of salt to the process stream. At low concentrations, usually, the protein solubility is increased (salting in). At higher concentrations, the protein solubility decreases and causes the protein to precipitate (salting out). The dissociated ions of the salt attract water molecules. Thereby, high salt concentrations disturb the solvation layer of water molecules around the protein and shield repulsion between surface charges of the same orientation. Salts with multiply charged anions are most effective in salting out proteins, while the cation is less important.

Calcium phosphate precipitation, even though salt-based, differs from the above-described principle of protein precipitation by salt. Calcium phosphate is poorly soluble in water above pH 6.5 (solubility product 3×10⁻⁷ M to 6×10⁻⁷ M, depending on the exact composition), with its solubility decreasing even further towards more alkaline pH values. Composition of calcium phosphate and the mechanism of its precipitation have been investigated in detail. Calcium phosphate in general, and hydroxyapatite in specific, are important role players in biological, geological and industrial processes. Calcium phosphate has been reported to co-precipitate viral vectors (reference 13) and has been described for DNA precipitation in antibody purification (reference 11). In addition to DNA precipitation, also a reduction of host cell proteins (HCP) was observed. With regard to the mechanism for protein precipitation by calcium phosphate, it was speculated on either co-precipitation with DNA or electrostatic interaction with the charged precipitate.

In a particularly preferred embodiment of the method for continuous recovering of a protein from a fluid in accordance with the present invention, in the protein separation step the protein in the fluid is precipitated using calcium phosphate. In this embodiment, the protein precipitation step preferably comprises adding calcium ions and phosphate ions to the fluid in accordance with the present invention. In a preferred embodiment, calcium ions are added to a final concentration of between 10 mM and 50 mM, preferably between 10 mM and 30 mM, more preferably 10 mM and 20 mM, most preferably about 15 mM. In another embodiment that can be combined with the previous embodiment, phosphate ions are added to a final concentration of between 1 mM and 10 mM, preferably between 1 mM and 5 mM, more preferably between 1 mM and 3 mM, most preferably about 2 mM. As will be clear to the skilled person, the calcium and/or phosphate ions are generally added as part of a solution that comprises calcium or phosphate ions and may comprise further ions. A suitable solution to add calcium ion is, e.g., a solution of CaCl₂*2H₂O in water. A suitable solution to add phosphate ion is, e.g., a solution of Na₂HPO₄ in water.

Alternative salts for precipitation in accordance with the present invention could include magnesium or zinc instead of calcium in combination with phosphate. Besides the mentioned ones, phosphate forms poorly soluble or insoluble salts with other divalent cations as for instance barium, cadmium, copper, lead and nickel. A suitable cation could be chosen based on considerations for patient health (e.g., toxicity when the protein to be recovered is a biopharmaceutical drug) and aspects with regard to the process of the invention, f.i. removability and process performance.

When a precipitant is used in the protein precipitation step of the method for continuous recovering of a protein from a fluid in accordance with the present invention, it is advantageous to ensure efficient mixing of the precipitant (e.g., calcium phosphate), and/or the components forming the precipitant (e.g., calcium ions and phosphate ions) with the fluid comprising the protein to be recovered, in order to ensure efficient precipitation of the protein. Thus, in a preferred embodiment of the method for continuous recovering of a protein from a fluid in accordance with the present invention, the protein precipitation step comprises mixing the fluid comprising the protein and the precipitant. Preferably, this mixing is performed in at least one reactor selected from the list consisting of a continuous stirred tank reactor (CSTR), a tubular reactor (TR), a segmented flow reactor, and an impinging jet reactor. In one embodiment, the mixing may be performed sequentially in several reactors, e.g. in a tubular reactor (TR) and in a continuous stirred tank reactor (CSTR). However, most preferably the mixing is performed in a continuous stirred tank reactor (CSTR).

When precipitating a protein by adding a precipitant, the difference in solubility between two or more solutes that occurs upon addition of a precipitant is exploited. The precipitants are usually added as concentrated stock solutions. Consequently, efficient mixing is advantageous to ensure homogenous conditions throughout the product stream. Therefore, it is preferable that the reactor for mixing the fluid comprising the protein and the precipitant in accordance with the present invention provides for such efficient mixing. Additionally, it is preferable that the reactor provides for sufficient contact or residence time in order for the precipitation process to be completed. Depending on the kinetics and characteristics of the process stream and the precipitant stock solution, a suitable reactor can be chosen. In literature continuous crystallization outweighs continuous precipitation. However, the basic principles regarding mixing and residence time distribution requirements are comparable, which is why the reactors described for one of both can also be used for the other. Mixed-suspension mixed-product removal reactors are based on continuous stirred tank reactors (CSTRs). These reactors are well characterized and are used for a wide range of applications in the biotechnological and biopharmaceutical industry. They are used for the cultivation of cells, as hold and surge tanks, for conditioning in between unit operations and for viral inactivation. CSTRs are available in stainless steel with process solutions for cleaning in place (CIP) and sterilization in place. With the reduction of equipment size, due to continuous processing, single-use technology has become an option as well as an enabling technology. Especially, for lower working volume demands, as is the case in continuous processing, single-use CSTRs are a viable alternative to stainless steel vessels. Both stainless steel and single-use CSTRs share the advantage of straightforward sensor installation for monitoring of process parameters. Depending on the demand for mixing, different stirrer geometries and configurations are available. CSTRs are characterized by broad residence time distributions with long washout times. It was previously thought that broad residence times could be disadvantageous especially with regard to disturbances that might arise during the course of a campaign. However, they can also provide benefits if smoothening of concentration fluctuation from cyclic operations is required. The dimensionless residence time distribution (F curve) of a CSTR is given below by Equation 2, where the dimensionless residence time θ is given by Equation 3. In Equation 3 t is residence time and E is the mean residence time.

$$F = {e}^{- θ} \qquad ( Equation 2 )$$

$$θ = \frac{t}{\overline{t}} \qquad ( Equation 3 )$$

In contrast, to the broad residence time distributions intrinsic to a CSTR, tubular reactors (TRs; also referred to as plug flow reactors) are characterized by narrow residence time distributions. In principle, a tubular reactor, is an open tube or pipe, equipped with static mixers. The required residence time is provided, by using a sufficiently long reactor, depending on the process flow rate and the time required for the precipitation. There are no generalized models available to describe the RTD of a tubular reactor. One option would be approximation by using a modified Gaussian peak and addition of an empirical factor. While PFRs have an advantage over CSTRs with regard to the narrowness of the RTD, they were previously thought to have several weaknesses in other areas. Sensors for online monitoring of the process are more difficult to install. At low flow rates, which correspond, to small scale processes, precipitates might settle within the TR or might be retained by static mixers. Furthermore, the lack of experience with TRs in the industry is expected to hamper implementation and acceptance of such reactors in production processes. Another flow-based reactor is the segmented flow reactor. The flow of the process stream is segmented by a second immiscible phase, which can be liquid or gaseous. Due to the lack of installations in the void of the reactor, the risk of clogging is significantly reduced, when compared to TRs equipped with static mixers. Furthermore, impinging jet mixers or reactors have been described for crystallization. Depending on the application, these reactors can be designed open or closed. In the closed, confined geometry, the level of control over the flow direction is higher and higher jet velocities can be realized. At the same time the confined geometry is more likely to clog than an open configuration.

The present inventors have surprisingly found that the pH of the fluid before precipitating the protein of the invention has a significant influence on the efficiency of protein precipitation. Thus, in a preferred embodiment of the method for continuous recovering of a protein from a fluid in accordance with the present invention, the pH of the fluid before precipitating the protein is adjusted to a pH of between 8.5 and 9.0, preferably to a pH of about 8.75. The skilled person will be aware of suitable substances (e.g., acids, bases) that can be used for adjusting the pH.

The stability of some proteins, such as Factor VIII, is distinctively reduced when the pH drops to below 6.5, or even to below 6.0, while precipitating the protein in the protein precipitation step in accordance with the present invention. Therefore, in a preferred embodiment of the method for continuous recovering of a protein from a fluid in accordance with the present invention, the pH of the fluid after precipitating the protein is between 6 and 7.5, preferably between 6.5 and 7, most preferably about 6.5.

The method for continuous recovering of a protein from a fluid in accordance with the present invention comprises a protein separation step of separating the precipitated protein from the fluid. The protein separation step is a solid-liquid separation step wherein a solid (i.e., the precipitated protein) is separated from a liquid (i.e., the fluid in accordance with the present invention). In one embodiment in accordance with the present invention, in the protein separation step a plate settler for protein separation, continuous tangential flow filtration or fluidized bed centrifugation is used for separating the precipitated protein from the fluid. In contrast to dead-end filtration, where there is only flux across the membrane, there is an additional flux parallel (tangential) to the membrane in tangential flow or cross-flow filtration. Thereby, repulsive forces summarized under the term “concentration polarization” are reduced. This mode of operation also significantly reduces compacting of a given precipitate at the membrane surface and thus allows retaining the floc structure (cf. references 5 and 6). Similarly, the floc structure is at least partially conserved in fluidized bed centrifugation. Typically, single-use chambers are used, where the suspension to be separated enters at the outer end of the chambers and clarified fluid leaves the chambers close to the rotor center. Due to this mode of operation a balance between the fluid flow towards the center and the centrifugal force from the center is established. Thereby, the solids are kept in a fluidized bed during the centrifugation and compacting is significantly reduced in comparison to classical centrifugation approaches (cf. reference 14).

In a particularly preferred embodiment in accordance with the present invention, in the protein separation step a plate settler for protein separation is used for separating the precipitated protein from the fluid. Thus, in this embodiment, the protein separation step is a step of separating the precipitated protein from the fluid using a plate settler for protein separation.

In the method for continuous recovering of a protein from a fluid in accordance with the present invention the molecular weight of the protein to be recovered is not particularly limited. However, the present inventors have surprisingly found that the method is suitable also for recovering large proteins. Thus, in a preferred embodiment, the protein to be recovered has a molecular weight of 250 kDa or more, preferably of 500 kDa or more, most preferably 1 MDa or more.

In the method for continuous recovering of a protein from a fluid in accordance with the present invention the concentration of the protein to be recovered in the liquid of the present invention before the protein precipitation step is not particularly limited. However, the present inventors have surprisingly found that the method is suitable also for recovering proteins at very low concentrations. Thus, in a preferred embodiment, the concentration of the protein in the fluid comprising the protein is below 20 μg/ml, preferably between 0.05 μg/ml and 20 μg/ml.

In the method for continuous recovering of a protein from a fluid in accordance with the present invention the type of protein is not particularly limited. The proteins in accordance with the invention include both recombinant proteins and proteins from other sources such as proteins obtained from (human) plasma, but preferably the proteins in accordance with the invention are recombinant proteins. Proteins in accordance with the invention include, without limitation, blood factors, immunoglobulins, replacement enzymes, growth factors and their receptors, and hormones. Preferred blood factors include factor I (fibrinogen), factor II (prothrombin), tissue factor, factor V, factor VII and factor Vila, factor VIII, factor IX, factor X, factor XI, factor XII, factor XIII, von Willebrand Factor (VWF), prekallikrein, high-molecular-weight kininogen (HMWK), fibronectin, antithrombin III, heparin cofactor II, protein C, protein S, protein Z, plasminogen, alpha 2-antiplasmin, tissue plasminogen activator (tPA), urokinase, plasminogen activator inhibitor-1 (PAI1), and plasminogen activator inhibitor-2 (PAI2). Preferred immunoglobulins include immunoglobulins from human plasma, monoclonal antibodies and recombinant antibodies. The proteins in accordance with the present invention may include functional polypeptide variants. The proteins in accordance with the invention are preferably the respective human or recombinant human proteins (or functional variants thereof).

In a preferred embodiment, the protein of the method for continuous recovering of a protein from a fluid is a blood coagulation factor. Blood coagulation factors in accordance with the present invention include factor I (fibrinogen), factor II (prothrombin), tissue factor, factor V, factor VII and factor Vila, factor VIII, factor IX, factor X, factor XI, factor XII, factor XIII. Preferred blood coagulation factors in accordance with the present invention are Factor VII (FVII) and Factor VIII (FVIII). The most preferred blood coagulation factor in accordance with the present invention is factor VIII. Preferably, the FVIII is human FVIII, which may be recombinantly produced, e.g., in CHO cells.

In another preferred embodiment in accordance with the present invention, the protein to be recovered is von Willebrand Factor (VWF). In a particularly preferred embodiment, the protein to be recovered is a protein complex comprising Factor VIII and von Willebrand Factor (VWF). This protein complex preferably comprises recombinant human Factor VIII and recombinant human von Willebrand Factor (VWF).

Hemophilia A is among the most well-known blood coagulation disorders, caused by a lack of Factor VIII (FVIII) co-factor activity. In healthy individuals, FVIII acts as a central co-factor in the blood coagulation cascade. FVIII is a trace plasma glycoprotein that is found in mammals and is involved as a cofactor of Factor IXa in the activation of Factor X. An inherited deficiency of Factor VIII results in the bleeding disorder haemophilia A, which can be treated successfully with purified Factor VIII. Such purified Factor VIII can be extracted from blood plasma, or can be produced by recombinant DNA-based techniques. Patients require life-long replacement therapy, which is often complicated by the development of FVIII inhibitors. Partially similar symptoms can be observed in cases of von Willebrand disease (VWD) resulting from a lack of von Willebrand factor (VWF). VWF and FVIII form a non-covalent (protein) complex that increases FVIII half-life time and protects it from premature activation. When the quantity or quality of VWF is compromised, the consequences for FVIII manifest in similar symptoms as for hemophilia A. Full length FVIII is a large glycoprotein of up to 330 kDa (based on SDS-PAGE). It consists of 2332 amino acids and circulates as a heterodimer in plasma. Full length FVIII consists of three A-domains bordered by short spacers, a B-domain and the two C-domains. Intracellular proteolysis produces the heterodimer found in plasma, consisting of a light and a heavy chain. Light and heavy chain are no longer covalently linked, but are associated via a metal ion bridging the A1 and A3 domains. The identity of the metal ion has remained unclear with the most likely candidates being copper and calcium. Upon proteolytic activation by thrombin the active hetero-trimer is formed, which is loosely associated via the metal ion and ionic interactions and therefore dissociates quickly. The B-domain of FVIII does not have any known functions. FVIII has very limited intrinsic in vitro and in vivo stability. This fact poses a major challenge on its production, recovery, purification and storage.

Von Willebrand factor (VWF) is a large multimeric plasma protein. The smallest subunit of VWF is comprised of pro-VWF-dimers from which the larger multimers are formed. The molecular weight ranges from roughly 500 kDa for dimers to above than 10.000 kDa for the largest variants. VWF has multiple functions, which can be briefly summarized as platelet binding, collagen binding and FVIII binding. In addition, VWF was reported to modulate memory immune responses to FVIII, which makes VWF an important factor in FVIII inhibitor formation. During VWF's physiological function, it binds collagen and is subsequently uncoiled upon exposure to shear stress. In its uncoiled form, VWF is able to bridge collagen and platelets and thereby VWF initiates and supports thrombus formation.

In one embodiment of the method for continuous recovering of a protein from a fluid in accordance with the present invention, the method further comprises a protein production step and a cell separation step before the protein precipitation step. In this embodiment, the protein production step is a step of culturing cells in a fluid, wherein the cells produce the protein and release the protein into the fluid, and the cell separation step is a step of separating the cells from the fluid comprising the protein. Thus, in this embodiment the method for continuous recovering of a protein from a fluid in accordance with the present invention comprises the following steps: a protein production step of culturing cells in a fluid, wherein the cells produce the protein and release the protein into the fluid; a cell separation step of separating the cells from the fluid comprising the protein; a protein precipitation step of precipitating the protein in the fluid; and a protein separation step of separating the precipitated protein from the fluid. All of these steps are performed in an integrated process. In a preferred embodiment, all of these steps are performed continuously.

In one embodiment, the cell separation step, the protein precipitation step and/or the protein separation step is/are performed at a temperature of between 0° C. and 8° C., preferably of between 2° C. and 8° C. In a preferred embodiment, all of the cell separation step, the protein precipitation step and the protein separation step are performed at a temperature of between 0° C. and 8° C., preferably of between 2° C. and 8° C.

In the embodiment of the method in accordance with the present invention comprising a protein production step and a cell separation step, the fluid is preferably a cell culture medium. As will be clear to a person skilled in the art, when cells are cultured in cell culture medium the composition of the cell culture medium changes, because cells secrete product and byproducts into the medium an consume nutrients. The term cell culture medium as used herein refers to cell culture medium before and after cells have been cultured therein, i.e. to both “fresh” and “spent” cell culture medium, respectively. Suitable cell culture media depend on the type of protein-producing cell that is used in this embodiment, and will be known to the person skilled in the art.

The cells that may be used in the method for continuous recovering of a protein from a fluid in accordance with the present invention are not particularly limited. However, preferably the cells are mammalian cells, such as Chinese hamster ovarian (CHO) cells, baby hamster kidney (BHK) cells, or human embryonic kidney (HEK) cells. In a particularly preferred embodiment, the cells are CHO cells.

Mammalian cells are routinely used to produce recombinant proteins (e.g., biopharmaceutical drugs) that may be secreted into cell culture medium (also referred to as cell culture broth fluid) and can eventually be recovered, e.g., to be formulated as a pharmaceutical composition. However, in order for cells to be capable of producing a recombinant protein, the cells need to comprise the respective genetic information. Accordingly, in a preferred embodiment in accordance with the present invention, the cells comprise genetic information encoding the protein to be recovered (e.g., a biopharmaceutical drug), so that the cells are capable of producing said protein.

The present invention is directed to a continuous method. Thus, it is preferred that, in the protein production step, also the culturing of cells is performed continuously. Continuous cell culturing processes include perfusion culture, turbidostat culture and chemostat culture. Thus, in a preferred embodiment of the present invention, in the protein production step the cells are cultured in a perfusion reactor, a turbidostat reactor or a chemostat reactor. In a particularly preferred embodiment, the cells are cultured in a chemostat reactor.

In the cell separation step any protein-producing cells that are carried over from the protein production step and therefore still comprised in the fluid (e.g., in the spent cell culture medium) in accordance with the present invention are separated and thereby removed from the fluid. Thus, this step is a solid-liquid separation step wherein a solid (i.e., the cells) is separated from a liquid (i.e., the fluid in accordance with the present invention, e.g., the cell culture medium). In a preferred embodiment, in the cell separation step also cell debris that may be carried over from the protein production step is separated and thereby removed from the fluid. In another embodiment that can be combined with the aforementioned embodiment, cell debris can be removed by filtration.

In a particularly preferred embodiment, in the cell separation step a plate settler for cell separation is used for separating the cells from the fluid comprising the protein. Thus, in this embodiment, the cell separation step is a step of separating the cells from the fluid using a plate settler for cell separation.

The “plate settler for protein separation” and the “plate settler for cell separation” in accordance with the present invention is any plate settler that is suitable for the indicated purpose, i.e. suitable for protein separation or suitable for cell separation, respectively. Preferably, the plate settlers in accordance with the present invention are inclined plate settlers. Examples of inclined plate settlers that can be used in the present invention are disclosed in US 2012/0302741 A1, U.S. Pat. No. 2,793,186 A1, 753,646 A1, and US 2002/0074265 A1, the contents of which are hereby incorporated in their entireties.

Plate settlers as well as bottom sections that may be connected to such plate settlers and which are particularly preferable for use in accordance with the present invention are described in the following. Since many of the optional embodiments of the “plate settler for protein separation” and the “plate settler for cell separation” in accordance with the present invention as well as of the bottom sections that may be connected to these plate settlers are identical, in the following it is only referred to “plate settler” and “bottom section” in general, without differentiating between the “plate settler for protein separation” and the “plate settler for cell separation” in accordance with the present invention as well as the corresponding bottom sections. However, unless indicated otherwise, all embodiments described in the following with reference to a “plate settler” and/or a corresponding “bottom section” are embodiments of the “plate settler for protein separation” and of the “plate settler for cell separation” in accordance with the present invention as well as of the corresponding bottom section. In one embodiment, the “plate settler for protein separation” and the “plate settler for cell separation” as well as the corresponding bottom sections in accordance with the present invention comprise some or all of the following features, such that they are (structurally) identical. However, in another embodiment in accordance with the present invention the “plate settler for protein separation” and the “plate settler for cell separation” and/or their corresponding bottom sections differ in one or more features, e.g. the “plate settler for protein separation” or its bottom section may comprise one or several of the following features, whereas the “plate settler for cell separation” or its bottom section does not comprise these features, or vice versa.

Inclined plate settlers can be used for separating a component from a fluid, i.e. in the present invention for separating precipitated protein or cells from the fluid in accordance with the invention. The sedimentation plates, on which the component to be separated can settle, of an inclined plate settler extend in an oblique rather than in the vertical direction, i.e., in a direction that is slanted with respect to the direction of gravity. A fluid is supplied to such a plate settler at its bottom end with a sufficiently high pressure such that the fluid flows upwards along the settler's sedimentation plates. The solid component to be separated may, e.g., already be present in the supplied fluid in solid form. Alternatively, the component to be separated may, e.g., precipitate under the influence of gravity. The remainder of the fluid flows on and is eventually exhausted from an outlet at the top end of the plate settler. The separated component (e.g., a solid component such as precipitated protein or cells) is collected from the bottom end of the plate settler. The bottom end of the plate settler may be connected to a component, often referred to as a “bottom section”, sometimes also referred to as “receiving section”, comprising supply channels for supplying a fluid containing the component to be separated and collection channels for collecting the separated component.

An inclined plate settler may comprise several sedimentation plates. A separation process can thus simultaneously take place in each of the sedimentation plates. Because both fluid comprising the component to be separated is supplied and the separated component is collected at the bottom end of the plate settler, the separated component may get mixed into the newly supplied fluid and thus be carried back upwards along the plate settler. This may lower the efficiency of the separation process. Therefore, in a particularly preferable embodiment in accordance with the present invention, the plate settler in accordance with the present invention is connected to a specially designed bottom section. The plate settler (which may be part of an assembly) as well as the specially designed bottom section that are preferably used in accordance with the present invention are described in the following disclosure:

Aspects of the present disclosure relate to a bottom section for being connected to an assembly for separating a solid component from a fluid, said assembly including an inclined plate settler with at least one sedimentation channel for letting a solid component to be separated settle, the plate settler comprising a lower portion and an upper portion, and the at least one sedimentation channel extending from the lower portion to the upper portion, wherein the bottom section is configured to be connected to the lower portion of the inclined plate settler.

The term “bottom section” is in this context not to be understood to imply that the bottom section necessarily is to be positioned at the “bottom” of an assembly in use and/or that the assembly rests on the bottom section (such that it would play the role of a “foot part”). The bottom section may or may not be at the bottom. In other words, the bottom section itself may, e.g., rest on another component positioned partially or fully below the bottom section. The bottom section may or may not constitute a foot member on which the assembly partially or fully rests, depending on the embodiment(s) in question.

The disclosure encompasses separately formed bottom sections that are (directly or indirectly) connectable to an inclined plate settler. The disclosure however also encompasses assemblies with bottom sections that are a part of a larger, integrally formed part (e.g., the bottom section may be made as one piece together with another component of an assembly).

The bottom section may comprise at least one inlet channel for feeding a fluid comprising the solid component to be separated to the plate settler, and at least one collection channel for collecting a settled solid component descending from the at least one sedimentation channel. The solid component may be collected as such or it may be collected in a suspended form, forming part of fluid. The solid component may already be present in solid form in the supplied fluid, or it may precipitate from the fluid in the plate settler. The collection channel may also be used to collect a fluid component (e.g., a heavier component) of a fluid supplied to an assembly comprising a plate settler.

Said at least one inlet channel and said at least one collection channel are fluidly separated from each other. By being “fluidly separated” it is meant that there is no direct fluid connection between the inlet channel and the collection channel in the bottom section. For example, a wall in the bottom section may separate the inlet channel and the collection channel. However, an indirect fluid connection (e.g., via a sedimentation channel in an assembly connected to the bottom section) may of course be present. The latter is not excluded by the absence of “being fluidly separated”, in accordance with the terminology used in this context.

The inlet channel and the collection channel may be connectable to the at least one sedimentation channel of an assembly to which the bottom section is connectable, to form fluid connections between said at least one inlet channel and said at least one sedimentation channel and between said at least one collection channel and said at least one sedimentation channel, respectively.

The fluid separation between inlet channel and collection channel (i.e., the absence of a direct fluid communication) may promote a better control over the behavior of fluid flows in the bottom section. Specifically, turbulences arising from mixtures of fluid being supplied and the descending separated solid component (e.g., a precipitate) and/or a descending separated fluid (e.g., comprising a solid component to be separated) in the bottom section or by virtue of the bottom section may be lowered or even avoided. Also, less or no separated component may be mixed into newly supplied fluid. Thus, the efficiency of the separation process carried out with an assembly connected to the bottom section may be increased by the bottom section in accordance with these embodiments.

According to some embodiments, the bottom section is configured to be connected to an assembly with a plate settler comprising a plurality of sedimentation channels and separation plates separating neighboring sedimentation channels. The bottom section may comprise a plurality of inlet channels and a plurality of collection channels, wherein said at least one inlet channel and said at least one collection channel are fluidly separated from all remaining inlet and collection channels, respectively.

The number of inlet channels may be equal to or different from the number of collection channels. Likewise, the respective numbers of inlet channels and of collection channels may be equal to or differ from the number of sedimentation channels of an assembly, to which the bottom section is configured to be connected. For some embodiments, the number of inlet channels is identical to the number of collection channels and is also identical to the number of sedimentation channels so that the bottom section comprises one inlet channel and one collection channel per sedimentation channel. This may particularly increase the efficiency of the separation process of an assembly connected to the bottom section.

The flow connection between said at least one inlet channel and the corresponding sedimentation channel and said at least one collection channel and the corresponding sedimentation channel may be separate from fluid connections between all other sedimentation channels and all other inlet channels and collection channels, respectively. This way, turbulent flows and/or other flow disturbances in the bottom section associated with the pair of channels comprising said at least one inlet channel and said at least one collection channel and the corresponding sedimentation channel and other channel pairs may be lowered or even fully avoided. This may further increase the efficiency of an assembly connected to the bottom section.

The bottom section in accordance with some embodiments may comprise one individual inlet channel and one individual collection channel for at least 50% of the sedimentation channels of a corresponding assembly, to which the bottom section is configured to be connectable. This may increase the efficiency as the degree of pairing is high in the sense that the number of channels not associated with a corresponding paired channel is 50% or lower. This may allow to lower or to suppress associated turbulent flows or other flow disturbances associated with neighboring channels that are not separated in terms of belonging to different channel pairs.

Optionally, there may be provided one individual inlet channel and one individual collection channel for at least 75% of the sedimentation channels of a corresponding assembly, or for at least 95% of the sedimentation channels. This may further increase the efficiency, respectively.

In accordance with some embodiments, the bottom section may comprise one individual collection channel and one individual inlet channel for each of the plurality of sedimentation channels, wherein a separate fluid connection is formable for each corresponding pair of inlet channel and sedimentation channel and for each corresponding pair of collection channel and sedimentation channel, respectively. This may lead to a particularly high efficiency of the assembly comprising the plate settler combined with the bottom section. Specifically, disturbance flows associated with neighboring pairs of channels may be minimized and losses of a separated solid component may be kept low or even avoided.

According to some embodiments, the bottom section may be configured to be connected to an assembly oriented in a use position such that end portions of the inlet channels and end portions of the collection channels proximate to the plate settler extend in the direction of gravity. In other words, a connection portion of the bottom section to be connected to an assembly may be oriented with respect to the end portions of the inlet channels and collection channels, respectively, such that when the connection portion is oriented with respect to the direction of gravity in the state of connection between assembly and bottom section ready for use, the end portions extend in the direction of gravity. According to some embodiments there may be an angle between the extension direction, when the bottom section is oriented as described, and the direction of gravity. The angle may lie in a range of 0° to 15°, optionally between 0° and 10°, or even between 0° and 5°. This may further increase efficiency.

An extension direction identical or similar to the direction of gravity (i.e., a vertical direction) of the end portions may promote similar or even equal hydrostatic pressures in different supply channels and/or collection channels, respectively. This means that a homogeneous use of an apparatus with a plate settler connected to the bottom section may be promoted.

Bottom sections in accordance with some embodiments may comprise at least one wash fluid supply channel for supplying a wash fluid (or a different fluid) to a sedimentation channel or to a collection channel, said at least one wash fluid supply channel being fluidly separated from other wash fluid supply channels and from all inlet channels. Again, the fluid separation refers to no direct communication within the bottom section but does not exclude the possible presence of an indirect connection (e.g., via a sedimentation channel). Being fluidly separated from other wash fluid supply channels and from the inlet channels may lower or even avoid the occurrence of efficiency lowering flow disturbances such as, e.g., turbulences associated with neighboring channels.

One or several wash fluid supply channels provide the possibility to supply another fluid, for example, a wash fluid that may be used to promote the collection of a separated fluid or solid component (e.g., a precipitate). This may promote the efficiency of a separation process. For example, when a solid component tends not to be drained efficiently, possibly because there is a tendency to adhere to surfaces such as parts of a collection channel, supplying a wash fluid may play an efficient contribution to collect the solid component and to “wash” it down through one or several collection channels of the bottom section. A wash fluid may also promote the separation of a solid component and the (remainder of) a supplied fluid. This may be of importance, for example, because the fluid phase may be of high value and/or as it may contain impurities, which one wants to get rid of. The use of a wash fluid is optional in the sense that removing bound or adhering solids may also be accomplished without the application of a wash fluid.

The at least one wash fluid supply channel and the at least one collection channel corresponding to the same sedimentation channel may be fluidly connected, for example, by an opening in a wall portion shared by said wash fluid supply channel and said collection channel. The fluid connection may be direct in the sense that the fluid connection may exist within the bottom section. This may inhibit or even prevent a supplied wash fluid accidentally being guided along the sedimentation channel and being drained out of the top end. It may also lower the amount of wash fluid being transported upward along the plate settler and being drained at the top end.

The fluid connection between fluid supply channel and collection channel in the bottom section may increase the efficiency of a process of washing out a separated fluid or solid component and to collect it via the collection channel(s). It may also additionally increase the flow efficiency by inhibiting or preventing flow disturbances, because a wash fluid may directly be guided towards (a) collection channel(s).

The bottom section in accordance with some embodiments may comprise at least one intrachannel distributing portion for evenly distributing a fluid flow through a part of a first channel proximate to a corresponding sedimentation channel over at least one direction of extension across the cross-section of said particular channel. The first channel may be directly adjacent to the sedimentation channel to be connected to it, or there may be a further component in-between. The intrachannel distributing portion may increase the efficiency of the use of an apparatus with a plate settler because it may, e.g., increase the homogeneity of the load applied to the associated sedimentation channel in question.

Said first channel is an inlet channel or a collection channel or a wash fluid supply channel. An intrachannel distributing portion may, more generally, be provided to one or several inlet channels and/or one or several collection channels and/or one or several wash fluid supply channels. For some embodiments, there is one intrachannel distributing portion for each inlet channel, one intrachannel distributing portion for each collection channel, and one intrachannel distributing portion for each wash fluid supply channel present. This may increase the efficiency of the bottom section in particular, as it may promote a particularly even flow distribution over all of the mentioned channels of the bottom section, both for fluids supplied to a connected assembly as well as for fluids/components drained (collected) therefrom.

The bottom section in accordance with some embodiments may comprise at least one interchannel distributing portion for evenly distributing a fluid flow in the direction to or the direction from a plate settler over a plurality of inlet channels and/or wash fluid supply channels and/or collection channels. There may be one or several interchannel distributing portions. One or several interchannel distributing portions may be provided for a part of or all of the inlet channels, one or several interchannel distributing portions may be provided for a part of or all of the collection channels, and one or several interchannel distributing portions may be provided for a part of or all of the wash fluid supply channels. However, several interchannel distributing portions may in this context also simply just be referred to as “an interchannel distributing portion”.

According to some embodiments, all inlet channels, all collection channels, and all wash fluid supply channels may be fluidly connected to an interchannel distributing portion. This may increase the efficiency of the bottom section in particular, as it may promote a particularly even flow distribution over all of the present channels, both for fluids supplied to a connected assembly as well as for fluids drained therefrom. According to some embodiments, a first interchannel distributing portion may be connected to all inlet channels, a second interchannel distributing portion may be connected to all collection channels, and a third interchannel distributing portion may be connected to all wash fluid supply channels. The terms “first”, “second”, and, “third” are just used as labels to distinguish between the three interchannel distributing portions.

The intrachannel distributing portion may connect an upper part of the first channel with a lower part of said first channel, wherein said upper part is located proximate to the corresponding sedimentation channel. The latter means that the upper part is closer to where the bottom section is to be connected to an apparatus including a plate settler than the lower part.

The lower part of the first channel may be split into two (or more) connecting channels of equal first cross-sections, and said connecting channels are optionally at least once further split into (two or more) respective connecting sub-channels with respective equal second cross-sections. With “equal first cross-sections” and “equal second cross-sections”, it is meant that all the cross-sections of the channels after the first split are equal, and likewise for the channels after the second split. Channels after a split may or may not have the same cross-sections as the channels before the split. The first cross-sections may thus be identical to or different from the respective second cross-sections, etc.

End portions of all of the connecting sub-channels after the respective last splits are connected to the upper part so as to be evenly distributed over a distributing direction. This may particularly promote the evenness of the distribution of fluid effected by the intrachannel distribution portion. The flow speed may or may not be kept substantially constant before and after a bifurcation (a point where a channel is split into two or more channels). According to some embodiments, all splits may double the number of channels. For other embodiments, split into three or more channels may be effected at a split point. Also different splitting numbers may be associated with different split points.

Subsequent splits may be effected at the same height when the channels are oriented to extend in a vertical direction. For example, the first split may be into two channels, and after the Nth set of splits (wherein each set is at a particular height), there may be 2N channels. The height differences between subsequent sets of splits may be identical or may be different. The cross-sections of all the channels may be identical. The cross-sections may be the same or different between each pair of channels corresponding to different stages in the bifurcated channel system with respect to the number of preceding sets of splits.

Each of the one or several interchannel distributing portions may comprise an upper portion to be connected to one or several inlet channels or one or several wash fluid channels or one or several collection channels, and a lower portion. The lower part may be split into two connection channels of equal first cross-section. Said connection channels may at least once further split into respective connection sub-channels of respective other equal cross-sections, wherein the first cross-sections are identical to or different from the respective other cross-sections, and wherein end portions of all of the connection sub-channels after the respective last splits are connected to the upper portion so as to be evenly distributed over a distributing direction. The distributing direction may be substantially or completely perpendicular to the extension direction of at least a part of the inlet channels and/or collection channels, and/or wash fluid supply channels.

This may particularly promote the evenness of the distribution of fluid effected by the interchannel distribution portion. The flow speed may or may not be kept substantially constant before and after a bifurcation (a point where a channel is split into two or more connection channels). According to some embodiments, all splits may double the number of channels. For other embodiments, splits into three or more channels may be effected at a split point. The number of splits at a split point may differ between split points or be the same for all of them.

Subsequent splits may be effected at the same height when the connection channels are oriented to extend in a vertical direction. For example, the first split may be into two connection channels, and after the Nth set of splits (wherein each set is at a particular height), there may be 2N channels. The height differences between subsequent sets of splits may be identical or may be different. The cross-sections of all the connection channels may be identical. The cross-sections may be the same or different between each pair of connection channels corresponding to different stages in the bifurcated channel system with respect to the number of preceding sets of splits.

According to some embodiments, the intrachannel distributing portion and the interchannel distributing portion may be connected. Serially combining the two types of distributing portions may particularly promote the evenness of flow distribution and thus be particularly beneficial to the efficiency of the bottom section (and thus of an apparatus connected to the bottom section). The intrachannel distributing portion may be configured to be arranged more proximately to the plate settler than the interchannel distributing portion.

There may be one interchannel distributing portion connected to several intrachannel distributing portions, one of the latter being connected to each inlet channel, and/or there may be one interchannel distributing portion connected to several intrachannel distributing portions, one of the latter being connected to each collection channel. There may be one interchannel distributing portion connected to several intrachannel distributing portions, one of the latter being connected to each wash fluid supply channel. When there is one intrachannel distributing portion for each inlet channel, one for each collection channel, and one for each wash fluid supply channel, respectively, and when the respective sets of inlet channel-associated intrachannel distributing portions, collection channel-associated intrachannel distributing portions, and wash fluid channel-associated intrachannel distributing portions each are preceded (in terms of the flow direction towards a connected apparatus) by one or several interchannel flow distributing portions, this may particularly promote the effectiveness and efficiency of the bottom section. In particular, it may particularly promote the evenness of the flow distribution towards an apparatus and thus also of flows in various sedimentation channels of an inclined plate settler.

All of the inlet channels and the collection channels may be provided in pairs in the sense that there may always be a collection channel for every inlet channel (and vice versa) such that one pair is associated with one or several corresponding sedimentation channels of a plate settler, respectively. All of the inlet channels, collection channels, and wash fluid supply channels may be provided as triplets.

All of the inlet channels may be fueled by one corresponding interchannel distributing portion each, all of the collection channels may be joined by one corresponding interchannel distributing portion. All wash fluid supply channels may be fueled by a respective corresponding interchannel distributing portion.

All of the inlet channels may be associated with one intrachannel distributing portion, all of the collection channels may be associated with one intrachannel distributing portion. All of the wash fluid supply channels may be associated with one intrachannel distributing portion. The association is to be understood to express that one respective intrachannel distributing portion is provided in the fluid flow path leading towards the corresponding inlet channel.

For some embodiments of the bottom section that comprise one or several intrachannel distributing portions and one or several interchannel distributing portions, a distributing direction of the intrachannel distributing portions may be a longitudinal extension direction of a cross-section of a connecting end part of the first channel to be located proximate to the plate settler. The first channel may also entirely extend in this mentioned direction. The distributing direction of the interchannel distributing portions may be perpendicular to the distributing direction of the intrachannel distributing portions. This may lead to a particularly efficient flow distribution pattern. In particular, it may allow for a compact build of the bottom section.

The one or several intrachannel distributing portion(s) may be fractal flow distributors. Likewise, the one or several interchannel distributing portion(s) may be fractal flow distributors. The fractal flow distributors split subsequently in several split levels and can be scaled up or down by increasing or decreasing the number of split levels.

Some embodiments of the bottom section are configured to be connected to an assembly that has bottom surfaces of neighboring sedimentation channels extending parallel to one another, said bottom surfaces including at least a part that is not inclined in any direction other than the direction of inclination of the sedimentation channels. Also the entire bottom surfaces may be inclined only in the direction of inclination of the sedimentation channels.

The angle of inclination of the sedimentation channels with respect to the direction of gravity may lie in a range of 5° to 85° (or 15° to 75°). This may promote (or even further promote) the efficiency of a separation process. According to some embodiments, the angle lies in a range of 50° to 70°, optionally in a range of 55° to 65°, and optionally in a range of 58° to 62°. An angle within these increasingly narrower ranges may increasingly further promote the efficiency of a separation process.

Another aspect of this disclosure relates to an assembly for separating a solid component from a fluid. The assembly may comprise an inclined plate settler with a lower portion, an upper portion, and at least one sedimentation channel for letting a solid component to be separated settle. The sedimentation channel may extend from the lower portion to the upper portion.

The plate settler may be an inclined plate settler. It may be configured to be oriented during use such that the at least one sedimentation channel extends from the lower portion to the upper portion in a direction that is inclined with respect to the direction of gravity. The at least one sedimentation channel of the plate settler may be connected to a fluid outlet for draining a rest fluid at the upper portion and connected to a bottom section according to any one of the previously embodiments at the lower portion. Rest fluid, from which a fluid (or only a solid component) to be separated has been partially or fully separated, may be drained from the upper portion through the fluid outlet.

The assembly may comprise a plurality of sedimentation channels for letting a solid component to be separated settle, said sedimentation channels extending from the lower portion to the upper portion, and the plate settler may further comprise separation plates separating neighboring channels. The plate settler may be configured to be oriented during use such that the separation plates do not overlap in the direction of gravity. The separation plates may be oriented in the direction of gravity in the sense that they are vertically extending separation walls between neighboring sedimentation channels, when the assembly is installed such that it is oriented for use.

The plurality of sedimentation channels may be connected to at least one fluid outlet for draining a rest fluid at the upper portion. The plurality of sedimentation channels is connected to a bottom section according to any one of the previous claims at the lower portion. Each sedimentation channel of said plurality may be connected to one or several inlet channel(s) and one or several collection channel(s), and it may further also be connected to one or several wash fluid supply channel(s). According to some embodiments, a one-to-one correspondence between pairs of inlet and collection channels and one sedimentation channel may be realized, and according to some embodiment there may be one triplet, consisting of one inlet channel, one collection channel and one wash fluid supply channel, for one sedimentation channel.

The width of sedimentation channels may generally for embodiments of the assembly in accordance with the present disclosure lie in a range of 5 cm to 200 cm, optionally a range of 40 cm to 150 cm. The height of settling plates (the bottoms of the sedimentation channels) may generally lie in a range of 10 cm to 200 cm. The distance between two settling plates may generally lie in a range of 0.3 cm to 10 cm.

The number of fluid outlets per cm plate width (after a last split of a flow distributor located closest to the plate settler) may lie in a range of 0.2 outlets/cm to 2 outlets/cm, optionally in a range of 0.5 outlets/cm to 1 outlet/cm.

The cross-section in longitudinal direction of fluid channels of the flow distributors of a bottom section in accordance with the present disclosure may be (at least partially) square shaped or of rectangular shape or circular shape.

According to some embodiments, the bottom section according to any one of the embodiments described herein may be used with an assembly according to any one of the embodiments described herein (in so far not incompatible), such that a relative difference between hydrostatic pressures in different sedimentation channels does not exceed a threshold of 10%. Optionally, the difference does not exceed a threshold of 5%, and optionally it does not exceed a threshold of 3%. These thresholds may (to an increasing degree with a lower threshold value) ensure very similar (or even substantially or fully identical) hydrostatic pressures in different sedimentation channels. This promotes a homogeneous and equilibrated use of the assembly and thus a higher efficiency, because it may make optimal use of the assembly's capacity.

According to some embodiments of the use of an assembly, said use comprises supplying a fluid comprising a solid component to be separated to the plate settler through the at least one inlet channel, and a wash fluid through the at least one wash fluid supply channel, wherein a density of the wash fluid is equal to or higher than a density of the fluid comprising the solid component to be separated. This may increase the efficiency of the desired separation process. It may also lower or even avoid losses of wash fluid as the tendency of wash fluid accidentally being transported up the sedimentation channel (and possibly even being drained through a top end outlet) may be lowered.

The bottom section/plate settler/assembly described above may be used for separating solid components from a fluid. Said separation of solid components from a fluid may comprise a step of feeding fluid comprising the solid components to the at least one inlet channel of the bottom section in accordance with the present disclosure; a step of letting the solid components settle; a step of draining (i.e., collecting) the rest fluid (i.e., the solid-depleted fluid); and a step of collecting the settled components through the at least one collection channel of said bottom section. Preferably, the step of letting the solid components (e.g., cells) to be separated settle is a step of letting the solid components settle in the at least one sedimentation channel of the inclined plate settler that is part of the assembly in accordance with the present disclosure. In the method for continuous recovering of a protein from a fluid in accordance with the present invention, these steps are performed as part of a continuous process, wherein several steps may be performed simultaneously (i.e., at the same time): Fluid comprising the solid components may be continuously fed to the bottom section and rest fluid may be continuously drained, so that the solid components comprised in the fed fluid may settle before the rest fluid is drained. The step of collecting the settled components may be performed intermittently, e.g., at regular intervals.

According to some embodiments, the solid components to be separated are precipitates. According to some embodiments, the solid components to be separated are cells. These cells may be freely suspended, or they may be adhering, e.g., to microcarriers.

When the solid components are cells, these cells may be capable of producing a protein, such as a coagulation factor. In such a case, the cells may have been cultivated in the fluid (e.g., in a cell culture broth fluid, also referred to as cell culture medium) before said fluid (including the cells contained therein) is fed to the bottom section in accordance with the present disclosure. During such prior cultivation, the cells may have produced the protein. Hence, in this embodiment in accordance with the present disclosure, the fluid that is fed to the bottom section in accordance with the present disclosure may contain said protein.

When performing the above separation in accordance with the present disclosure, the inventors have found that solid components (e.g., cells) that are contained in a fluid (e.g., in a cell culture broth fluid) can be efficiently separated from said fluid with minimal loss of any components that are dissolved in the fluid, such as proteins. Thus, in accordance with the method of the present disclosure, any components that are dissolved in the fluid can be efficiently harvested together with the solid-depleted fluid phase. Accordingly, the present disclosure provides an improved separation of solid components from a fluid.

For a better understanding of the present disclosure and to show how the same may be carried into effect, in the following embodiments of the bottom section/plate settler/assembly that is preferably used in the method for continuous recovering of a protein from a fluid in accordance with the present invention will be described by reference to the accompanying drawings.

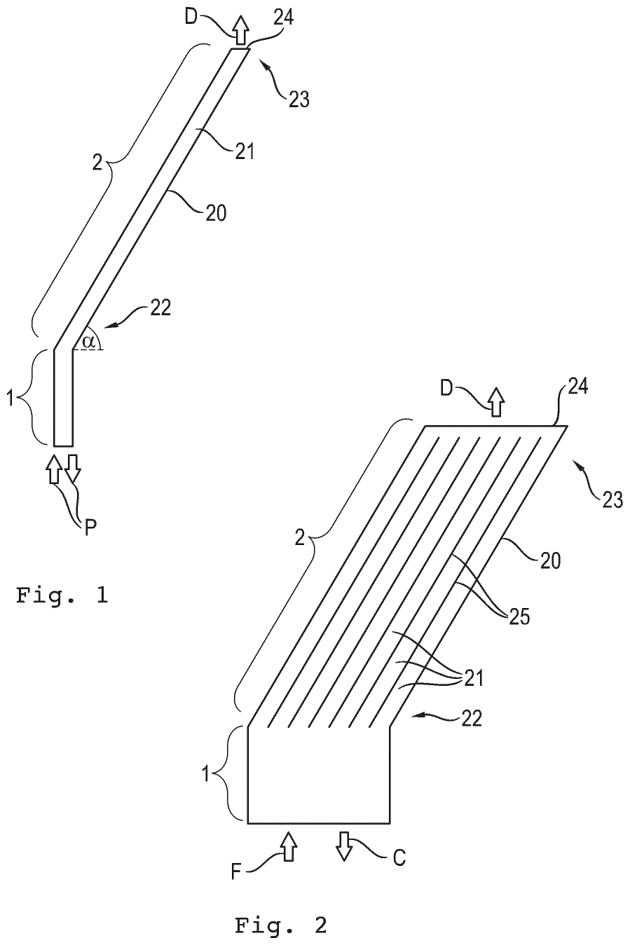

FIG. 1 depicts an embodiment of a bottom section 1 in accordance with the present disclosure. The bottom section 1 is connected to an embodiment of an assembly 2 for separating a solid component from a fluid in accordance with the present disclosure.

The assembly 2 includes an inclined plate settler 20. It is referred to as inclined because it extends at an angle α with respect to the direction of gravity (the vertical direction in FIG. 1).

This embodiment of the plate settler 20 includes one sedimentation channel 21 for letting a fluid to be separated (e.g., a solid component to be separated) settle. The inclined plate settler 20 has an inclination angle α that is adapted to the densities of the fluid fed to the plate settler 20 and to the density (specific weight, etc.) of the component to be separated (in this case: a solid component on the bottom of the sedimentation channel 20).

The angle α of inclination of the plate settler 20 with respect to the direction of gravity of various embodiments of assemblies and bottom sections in accordance with the present disclosure may lie between 5° and 85°.

The plate settler 20 comprises a lower portion 22 and an upper portion 23. The sedimentation channel 21 extends from the lower portion 22 to the upper portion 23. The bottom section 1 is connected to the lower portion 22. The upper portion 23 is connected to a fluid outlet 24. Rest fluid, from which the fluid (in this case: the precipitated solid component) has been (at least in part) separated, is drained from the upper portion 23 through the fluid outlet 24. The fluid leaving the outlet 24 (and its directions) is symbolized by the arrow D in FIG. 1 (“D” stands for “drain”).

Fluid (including the component to be separated) is fed to the assembly 2 through the bottom section 1 from the bottom end. The separated component is also collected through the bottom end. This is symbolized by the double arrow Pin FIG. 1.

The bottom section 1 of FIG. 1 is separable from the assembly 2. However, the disclosure also encompasses bottom sections 1 that are integrally formed together with the assembly 2 (assembly 2 and bottom section 1 are made as one piece). The connection between assembly 2 and bottom section 1 in accordance with some embodiments may be reversible, and it may be irreversible for other embodiments.

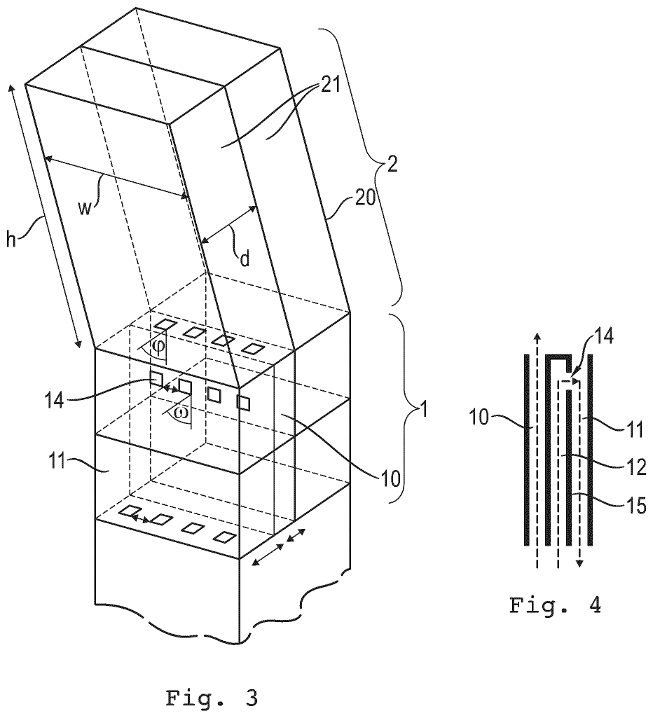

FIG. 2 depicts another embodiment of a bottom section 1 in accordance with the present disclosure. The bottom section 1 is connected to an embodiment of an assembly 2 for separating a solid component from a fluid in accordance with the present disclosure.

The assembly 2 includes an inclined plate settler 20. This embodiment of the plate settler 20 includes several sedimentation channels 22 for letting a component to be separated settle.

The plate settler 20 comprises a lower portion 22 and an upper portion 23. The sedimentation channels 21 extend from the lower portion 22 to the upper portion 23. The bottom section 1 is connected to the lower portion 22. The upper portion 23 is connected to a fluid outlet 24. Rest fluid, from which the fluid (in this case: the precipitated solid component) has been (at least in part) separated is drained from the upper portion 23 through the fluid outlet 24. The fluid leaving the outlet 24 (and its directions) is symbolized by the arrow D in FIG. 2 (“D” stands for “drain”).

Neighboring sedimentation channels 21 are separated by separating walls 25.

Fluid (including the component to be separated) is fed to the assembly 2 through the bottom section 1 from the bottom end. The arrow F symbolizes the fluid being fed (“F” stands for “fed”). The separated component is also collected through the bottom end. This is symbolized by the arrow C in FIG. 2 (“C” stands for “collect”).

The bottom section 1 of FIG. 2 is separable from the assembly 2. However, the disclosure also encompasses bottom sections 1 that are integrally formed together with the assembly 2 (assembly 2 and bottom section 1 are made as one piece). The connection between assembly 2 and bottom section 1 in accordance with some embodiments may be reversible, and it may be irreversible for other embodiments.

FIG. 3 is a schematic three dimensional perspective view of an embodiment of a bottom section 1 in accordance with the present disclosure. The bottom section 1 is connected to an embodiment of an assembly 2 for separating a solid component from a fluid in accordance with the present disclosure.

The assembly 2 comprises a plate settler 20. FIG. 3 shows only two sedimentation channels 21 in order not to clutter the schematic representation, however, the number of sedimentation channels 21 may be higher (e.g., a lot higher).

The width w of sedimentation channels 21 may generally for embodiments of the assembly 2 in accordance with the present disclosure lie in a range of 5 cm to 200 cm, optionally a range of 40 cm to 150 cm. The height h of the settling plates (the bottom surfaces of the sedimentation channels 21) may generally lie in a range of 10 cm to 200 cm. The distance d between two settling plates may generally lie in a range of 0.3 cm to 10 cm.

The settling plates (bottom walls) of the sedimentation channels 21 of this embodiment comprise stainless steel that is optionally electropolished (to a resolution of equal to or less than 0.8 μm). According to some embodiments, the settler plates consist of stainless steel. Alternatively, they may comprise or consist of a plastic such as acrylic glass (e.g., polymethyl methacrylate (PMMA) and/or polyethylene terephtalate glycol-modified (PETG)).

The bottom section 1 in accordance with this embodiment is made of stainless steel and/or plastics, and is assembled from layers. Alternatively, it can be made by additive manufacturing (e.g., 3D-printing). However, all of these features may be present in some embodiments and absent from others.

The bottom section 1 of FIG. 3 comprises several inlet channels 10 for feeding a fluid comprising the solid component to be separated to the plate settler 20. The bottom section 1 also comprises several collection channels for collecting a settled solid component descending from the sedimentation channels 21. Other embodiments comprise only one collection channel 11 and/or only one inlet channel 10.

The inlet channels 10 and the collection channels 11 are provided in pairs in the sense that there is one of each of these two channels connected to a corresponding sedimentation channel 21 of the plate settler 20.

Each of the inlet channels 10 and the collection channels 11 are connected to one corresponding sedimentation channel 21, to form fluid connections. The inlet channels 10 and the collection channels 11 are fluidly separated in the sense that there is no direct fluid connection between them within the bottom section 1. They are separated by a wall. An indirect fluid connection via the sedimentation channel 21, however, exists (this way, the separated solid component may return downward in FIG. 3 from the plate settler 20).

The feed angle φ between the inlet channels 10 and the sedimentation channels 21 is in this case 90°. Put differently, end portions of the inlet channels 10 proximate to the plate settler 20 extend in the direction of gravity. Moreover, also end portions of the collection channels 11 proximate to the plate settler 20 extend in the direction of gravity.

According to other embodiments, the angle φ may lie in a range of 5° and 90°, optionally in a range of 15° and 75°, or in a range of 30° and 60°. The angle φ may also be identical or similar to the inclination angle α of inclination of the plate settler 20. When the angle φ is smaller than 90°, the main part of the supply channel may, e.g., extend in the direction of gravity, and a portion proximate to the end (or the end portion) to be connected to a sedimentation channel may have a portion where the inclination of the supply channel changes. For example, there may be provided a bend (e.g., with an edge) in the supply channel, or the supply channel may comprise a curved portion, so that the angle of extension with respect to a horizontal plane transitions from 90° to an angle φ smaller than 90°.

The fluid separation (i.e., the absence of a direct fluid communication) between inlet channels 10 and collection channels 11 promotes a better control over the behavior of fluid flows in the bottom section 1. Specifically, turbulences arising from mixtures of fluid being supplied and the descending separated solid component (e.g., a precipitate) and/or a descending separated fluid (e.g., comprising a solid component to be separated) in the bottom section 1 or by virtue of the bottom section 1 may be lowered or even avoided. Thus, the efficiency of the separation process may be increased by the bottom section 1 in accordance with these embodiments.

The flow connection between the inlet channels 10 and the corresponding sedimentation channels 21 and the collection channels 11 and the corresponding sedimentation channels 21, respectively, is separate from fluid connections between all other sedimentation channels 21 and all other inlet channels 10 and collection channels 11, respectively. This way, turbulent flows and/or other flow disturbances in the bottom section 1 associated with the pair of channels comprising the respective inlet channel 10 and collection channel 11 and the corresponding sedimentation channel 21 and other channel pairs may be lowered or even fully avoided. This may further increase the efficiency of an assembly 2 connected to the bottom section 1.

The bottom section 1 of FIG. 3 comprises one individual collection channel 12 and one individual inlet channel 11 for each of the plurality of sedimentation channels 21, wherein a separate fluid connection is formed for each corresponding pair of inlet channel 10 and sedimentation channel 21 and for each corresponding pair of collection channel 11 and sedimentation channel 21, respectively. This may lead to a particularly high efficiency of the assembly 2 comprising the plate settler 20 combined with the bottom section 1. Specifically, flow disturbances associated with neighboring pairs of channels 10, 11, 21 may be minimized.

In order to keep the schematic representation of FIG. 3 simple, the figure does not distinguish between the collection channel 11 and respective corresponding wash fluid supply channels 12. The wash fluid supply channels 12 are located between the inlet channels 10 and the collection channels 12. Wash fluid is fed through the wash fluid supply channels 12 and is used to increase the efficiency of the draining of the separated component through the collection channels 11. FIG. 4 shows in more detail how the triplets of inlet channel 10, collection channel 11, and wash fluid supply channel 12 are configured.

The wash fluid supply channels 12 more generally may be used to supply a wash fluid to one or several sedimentation channels 21 or to one or several 12 collection channels directly. The wash fluid supply channels 12 are fluidly separated from other wash fluid supply channels 12 and from all inlet channels 10. This is shown, e.g., in FIG. 4.

Being fluidly separated from other wash fluid supply channels 12 and from the inlet channels 10 may lower or even avoid the occurrence of efficiency lowering flow disturbances such as, e.g., turbulences associated with neighboring channels 12. The fluid separation pertains to the bottom section 1 itself, but does not mean that there is no indirect fluid connection via, e.g., a connected plate settler 20.

The wash fluid may promote the efficiency of a separation process. For example, when a solid component tends not to be drained efficiently, possibly because there is a tendency to adhere permanently or temporarily to parts of a sedimentation plate or, e.g., to a collection channel 11, supplying the wash fluid may play a sufficient contribution to collect the solid component and to wash it out in one or several collection channels 11 of the bottom section 1.

As can be seen in FIG. 4, the corresponding wash fluid supply channels 12 and collection channels 11 (together corresponding to the same sedimentation channel 21) are fluidly connected by an opening 14 in a wall portion 15 shared by said wash fluid supply channel 12 and said collection channel 11. The fluid connection may be direct in the sense that the fluid connection may exist within the bottom section 1. This may inhibit or even prevent a supplied wash fluid accidentally being guided along the sedimentation channel 21 and being drained out of the top end. The fluid connection in the bottom section 1 may increase the efficiency of a process of washing out a separated fluid or solid component and to collect it via the collection channels 11. It may also additionally increase the flow efficiency by inhibiting or preventing flow disturbances, because a wash fluid may directly be guided towards the collection channels 11.

The openings 14 are also shown in FIG. 3. The angle ω of the wash fluid outlets (the openings 14) is in this case 90° with respect to the direction of gravity (the vertical direction in FIG. 3). It may alternatively lie in a range of 15° to 90° with respect to a horizontal direction, e.g., it may extend in the same (or a similar direction) as the principal direction of extension of the sedimentation channels 21 of the plate settler 20.

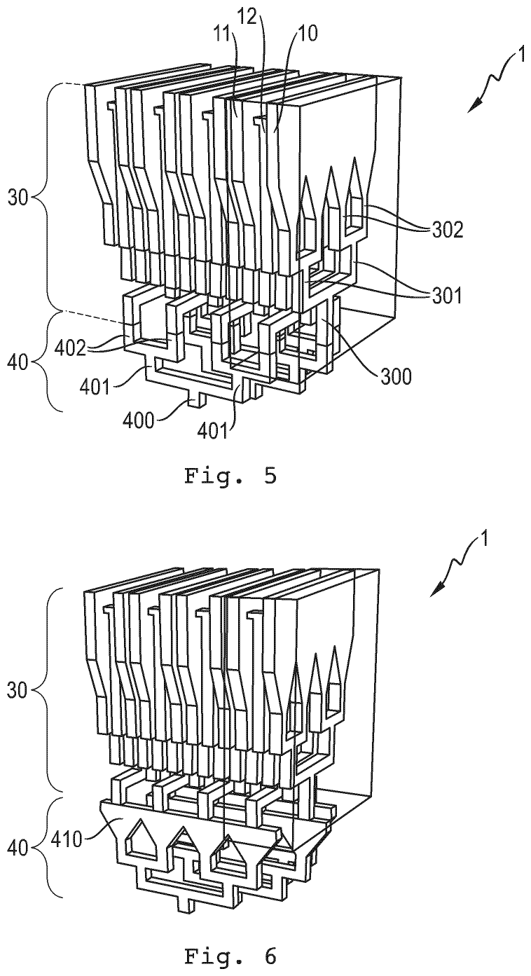

FIGS. 5 and 6 depict schematic three dimensional views of embodiments of a bottom section 1 in accordance with the present disclosure.

The bottom section 1 of FIG. 5 comprises an intrachannel distributing portion 30 for evenly distributing a fluid flow through the inlet channels 10, the collection channels 11, and the wash fluid supply channels 12, respectively. The intrachannel distributing portion 30 is a fractal flow distributor. The intrachannel distributing portion 30 may increase the efficiency of the use of an assembly 2 connected to the bottom section 1, because it may, e.g., increase the homogeneity of the load applied to corresponding sedimentation channels 21.

The intrachannel distributing portion 30 evenly distributes for all of the inlet channels 10, the collection channels 11, and the wash fluid supply channels 12. In the case of the collection channels 11, the even distribution is to be understood as a form of evenly collecting with respect to the entire diameter of an entire collection channel 11.

For every inlet channel 10, for example, the intrachannel distributing portion 30 comprises a channel 300 that is split into two channels 301, which are then again split into two channels 302 in the direction approaching the portion to be connected to an assembly 2 with a plate settler 20. This can be scaled up in accordance with the desired application and may be referred to as a fractal design of the flow distributor.

The embodiment of FIG. 5 comprises cone-shaped distributing portions which evenly distribute fluid exiting the channels 302 in order to reach the entire cross-section in width direction of the respective inlet channel 10 at a connecting portion to be connected to a plate settler 20.

For every collection channel 11, for example, the intrachannel distributing portion 30 comprises a channel 300 that is split into two channels 301, which are then again split into two channels 302 in the direction approaching the portion to be connected to an assembly 2 with a plate settler 20. This can be scaled up in accordance with the desired application and may be described as being associated with a fractal design of the flow distributor.

Analogous fractal channel arrangements are also provided for each of the collection channels 11 and each of the wash fluid supply channels 12. To avoid repetitions, reference is made to the explanation concerning the channels 300, 301, and 302 for the inlet channels 10.

The bottom section 1 of FIG. 5 also comprises an interchannel distributing portion 40 for evenly distributing a fluid flow in the direction to or the direction from a plate settler over the plurality of inlet channels 11 and over the wash fluid supply channels 12 and over the collection channels 11, respectively. This may further increase the efficiency of the bottom section 1, as it may promote a particularly even flow distribution over all of the present channels, both for fluids supplied to a connected assembly as well as for fluids drained therefrom.

In particular, the interchannel distributing portion 40 is a fractal flow distributor and comprises a distributing portion for all of the inlet channels 10, for all of the collection channels 11, and for all of the wash fluid supply channels 12.

For example, the channel 400 collects fluid from (all of) the collection channels 11. In the direction towards a plate settler 20 connected to the bottom section 1, the channel 400 is split into two channels 401, which are again split into two respective channels 402 each. This illustrates the fractal configuration of the flow distributor. Analogous structure exist for the interchannel distributing portion serving all of the inlet channels 10, and likewise for the interchannel distributing portion serving all of the wash fluid supply channels 12.

The interchannel distributing portion 40 and the intrachannel distributing portion 30 are connected in series, wherein the intrachannel distributing portion 30 is to be located closer to a connected plate settler 20 than the interchannel distributing portion 40.

An example is explained on how the two serially connected flow distributors work. For every collection channel 11, for example, an intrachannel distributing portion first homogeneously collects fluid (evenly over the cross-section of the collection channel 11). This is done by consecutive uniting of the channels leading from the connecting portion between assembly 2 and bottom section 1 towards the connecting part between the two flow distributors 30, 40. Then, an even collection, evened out over the different intrachannel distributing portions associated with the various collection channels 11, is effected over all of the collection channels 11 by the interchannel distributing portion. Analogous statements hold with respect to the inlet channels 10 and the wash fluid supply channels 12.

FIG. 6 depicts another embodiment of a bottom section 1 comprising an intrachannel distributing portion 30 and an interchannel distributing portion 40. The embodiment is similar to the embodiment of FIG. 5. Reference is therefore made to the explanations provided with regard to FIG. 5, and only differences will be discussed. The interchannel distributing portion 40 of FIG. 6 namely comprise cone-shaped distributing portions 410 at the part of the interchannel distributing portion 40 connected to the neighboring intrachannel disturbing portion 30. Some embodiments comprise these, whereas others do not. The cones are one of several aspects which may contribute to the evening effect of the flow distributor.

More generally, in the fractal flow distributors which are examples of interchannel distributing portions and/or intrachannel distributing portions of bottom sections 1 in accordance with the present disclosure, may comprise channels that are split into two (or more) connecting channels of equal first cross-sections, and said connecting channels are preferably at least once further split into (two or more) respective connecting sub-channels of respective other equal cross-sections. There may be one split, two splits, or several splits.

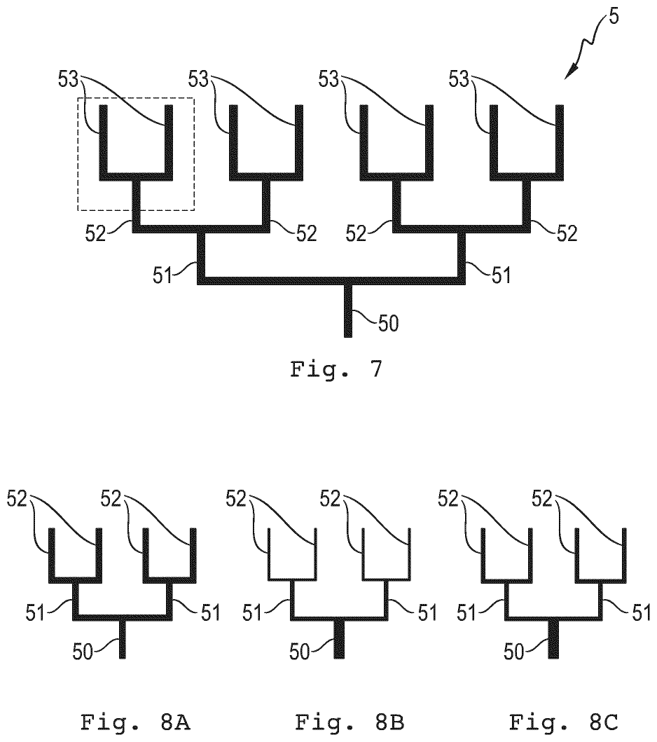

FIG. 7 illustrates an example of a flow distributor 5 with three split levels, wherein the splits always are a doubling of the number of channels. Concretely, the channel 50 is split into two channels 51, which are again split into two channels 52 each, wherein each of the channels 52 is again split into two respective channels 53. This can be scaled up as desired in order to scale up an assembly for separating a component of interest from a fluid.

A fractal fluid distributor 5 such as the one illustrated in FIG. 7 may be used for every single inlet channel 10, and/or for every single collection channel 11, and/or for every single wash fluid supply channel 12 of a bottom section 1 in accordance with the present disclosure. This way, the fluid distributor 5 may serve as a (or a part of a) intrachannel distributing portion 30.

The fractal fluid distributor 5 of FIG. 7 may in addition thereto or alternatively be used for several (or for all) inlet channels 10, and/or for several (or for all) collection channels 11, and/or for several (or for all) wash fluid supply channels 12. This way, the fluid distributor 5 may serve as a (or a part of a) interchannel distributing portion 40.

The flow distributor 5 of FIG. 7 is composed such that the cross-section of each channel after a split is identical to the cross-section of a channel before a split. In other words, the cross-section of channel 50 is equal to the cross-section of each of the channels 51, 52, and 53. Such a splitting scheme with equal cross-sections is also illustrated by FIG. 8A.

However, this disclosure encompasses other embodiments. FIG. 8B, for example, discloses a flow distributor splitting scheme, wherein the cross-section of channels is smaller after each split. In other words, in the case of FIG. 8B, the cross-section of channels 52 is smaller than the cross-section of channels 51, and the cross-section of the channels 51 is smaller than the cross-section of channel 50. In contrast, in the case of FIG. 8C, the cross-section is sometimes the same before and after a split, and sometimes it differs between before and after a split. Concretely, the cross-sections of the channels 51 and 52 are of equal size, whereas the cross-section of the channel 50 is larger.

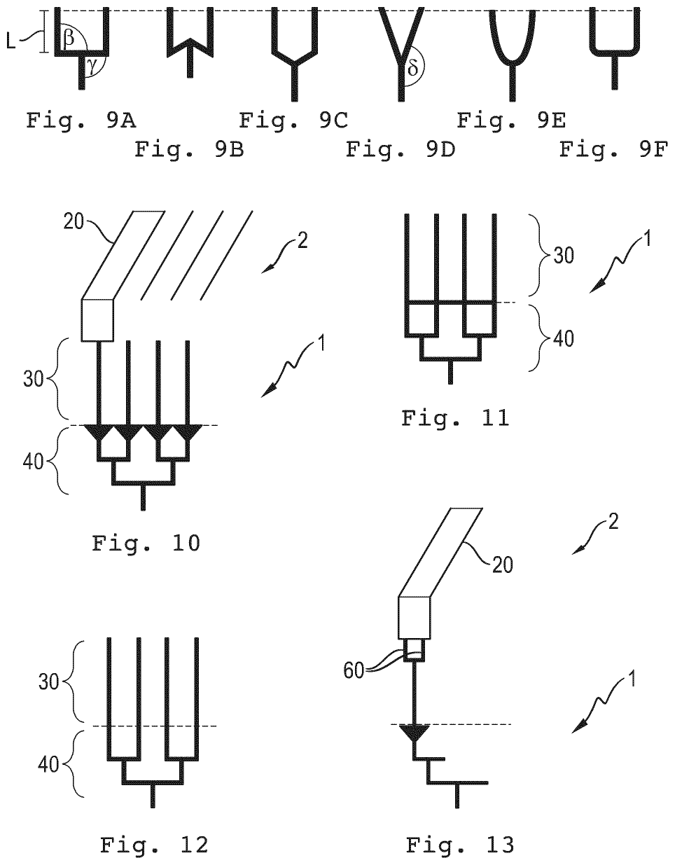

FIGS. 9A to 9F illustrates various possible split geometries that can be used in flow distributors being (part of) an interchannel and/or an intrachannel distributing portion of a bottom section 1 in accordance with the present disclosure.

The splits may be characterized, for example, by two angles β and γ. FIG. 9A shows a configuration of split where β=γ=90°. In the case of FIG. 9B, both β and γ are smaller than 90°. In the case of FIG. 9C, both β and γ are larger than 90°. FIG. 9D shows a case in which the angles β and γ are replaced by a geometry associated with a single angle δ. A split may also be formed by a curve rather than involving some sharp angles, as illustrated by FIG. 9E. In the case of FIG. 9F, two angles β and γ are 90°, but the edges are flattened out so that the shape in the corners is curved. All of these splits may be used as binary splits (splits into two channels) in flow distributors of bottom sections 1 in accordance with the present disclosure. However, also non-binary splits (e.g., splits into three, four, or more channels) may be used.

FIG. 10 schematically depicts two serially connected fractal flow distributors as an intrachannel distributing portion 30 and an interchannel distributing portion 40 of a bottom section 1 connected to an assembly 2 with an inclined plate settler 20. The intrachannel distributing portion 30 and the interchannel distributing portion 40 are rotated by 90° with respect to one another, so that the width directions are perpendicular to one another. Consequently, one can see the splitting up in stages of the interchannel distributing portion 40 in FIG. 10, whereas the components of the intrachannel distributing portion 30 appear as lines in FIG. 10.

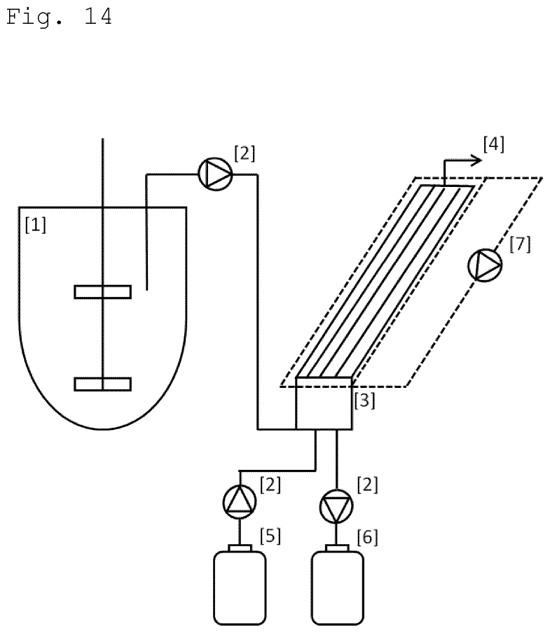

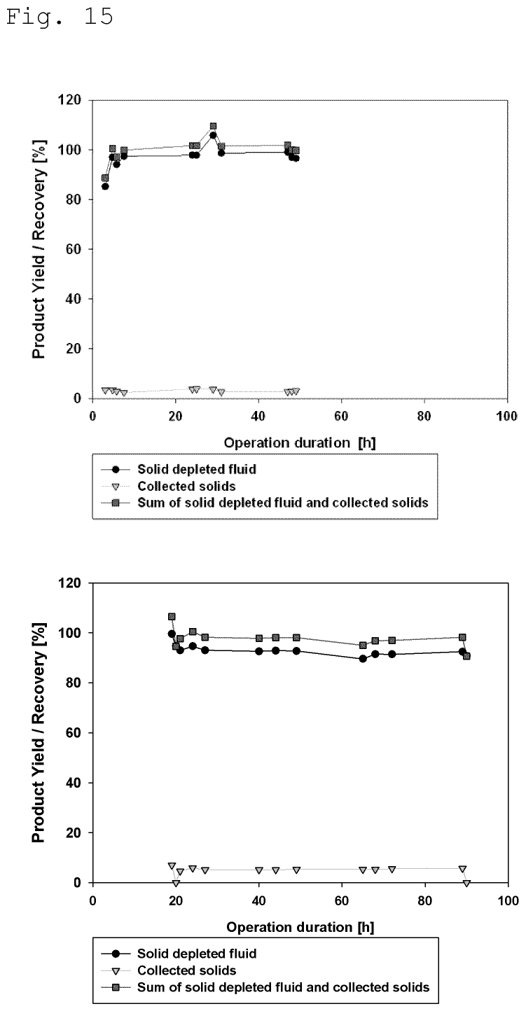

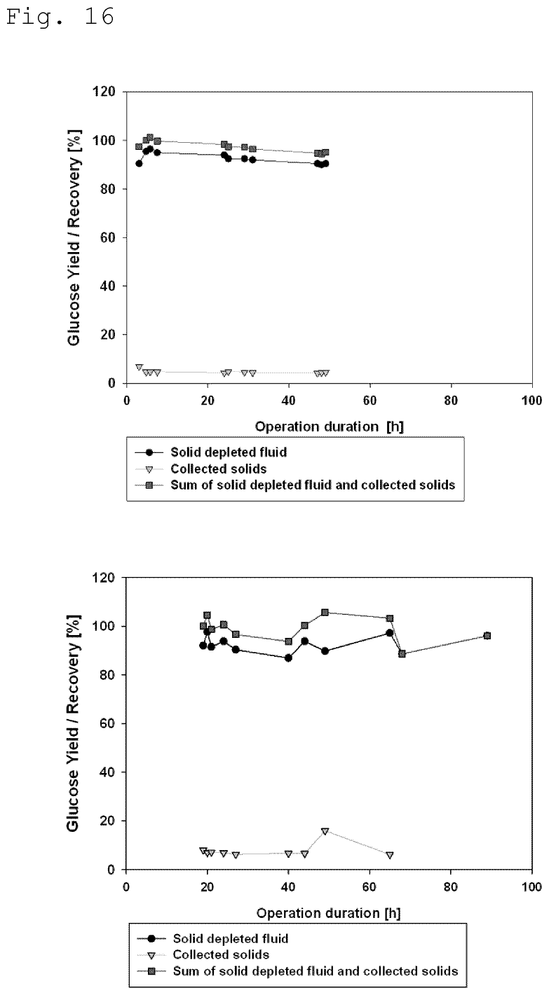

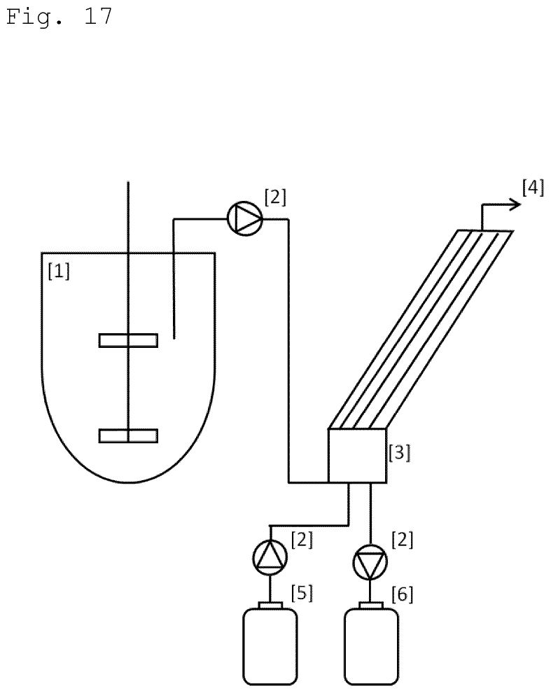

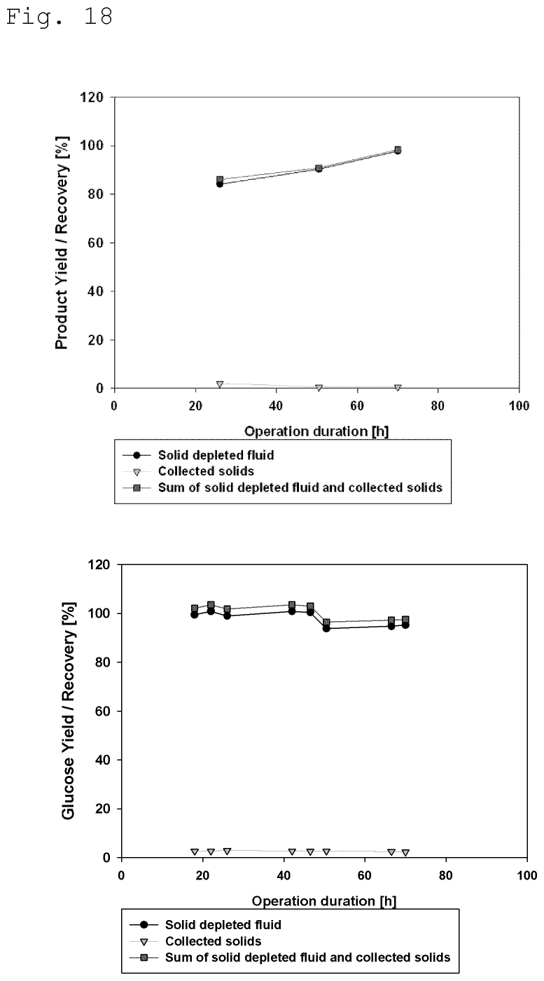

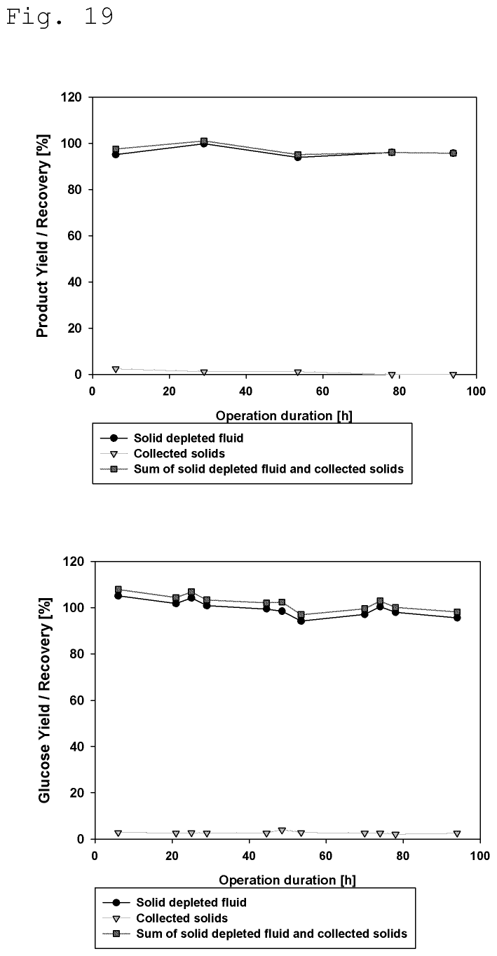

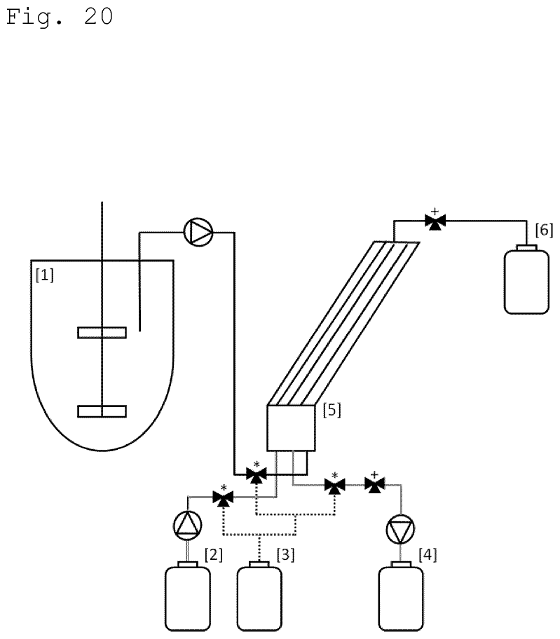

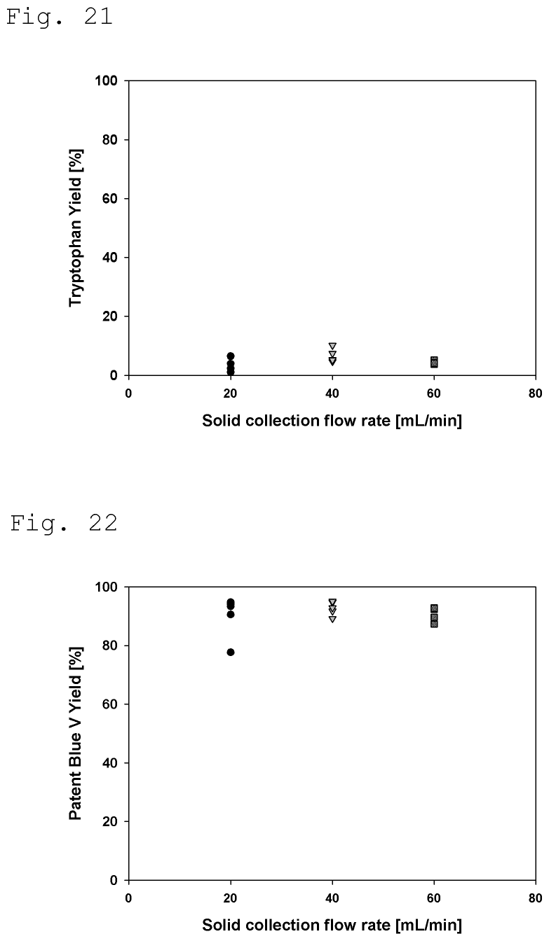

The connection between the two flow distributors may, as in the case of FIG. 10, be in the form of cone-shaped extensions so that one integral connecting zone is provided. Alternatively, the connection zone may be present but without any cone-shaped portions, as illustrated by FIG. 11. Another example is shown in FIG. 12, where there is no fluid connection between the different parts of the interchannel distributing portion 40 that are connected to an intrachannel distributing portion 30.

FIG. 13 shows another example of the serial connection of two fractal flow distributors as an intrachannel distributing portion 30 and an interchannel distributing portion 40, wherein there is a 90° rotation in-between (as described with respect to the assembly of FIG. 10). In the case of FIG. 13, another 90° rotation is effected within the intrachannel distributing portion 30, before the last split level. In other words, a split into two channels is provided in a perpendicular direction to the previous splits at the part of the intrachannel distributing portion 30 located closest to the plate settler 20 of the connected assembly 2. The last split into two channels 60 in a perpendicular direction may be particularly useful, for example, when very large solids are to be separated from a fluid, as the widths of the collecting zones may then be rather large. The width split in half may make the suctioning of solids from the collection zone more efficient.

Some embodiments of bottom sections 1 and/or assemblies 2 in accordance with this disclosure may be used such that a relative difference between hydrostatic pressures in different sedimentation channels does not exceed a threshold of 10%. Optionally, the difference does not exceed a threshold of 5%, and optionally it does not exceed a threshold of 3%. These thresholds may (to an increasing degree with a lower threshold value) ensure very similar (or even substantially or fully identical) hydrostatic pressures in different sedimentation channels. This promotes a homogeneous and equilibrated use of the assembly and thus a higher efficiency, because it may make optimal use of the assembly's capacity.

A maximum linear velocity in a channel of a flow distributor (of the intrachannel and/or interchannel distributing portion(s)) may be 1 ml/min/cm plate width of volumetric flow rate during solid removal (and wash flow), up to 50 ml/min/cm plate width. The Reynolds number of the fluid at the top outlets of the upper flow distributor (closest to the plate settler) may be lower than 2000. A length of a fluid channel of a flow distributor may be in the range of 0.5 cm to 5 cm.

The bottom section/plate settler/assembly of the present disclosure can be used for separating solid components (e.g., precipitated protein or cells) from a fluid. Said separation may comprise a step of feeding fluid comprising the solid components to the at least one inlet channel of the bottom section of the present disclosure; a step of letting the solid components settle; a step of draining (i.e., collecting) the rest fluid (i.e., the solid-depleted fluid); and a step of collecting the settled components through the at least one collection channel of said bottom section. Preferably, in the step of draining the rest fluid the rest fluid is not drained directly from the bottom section, but rather from other parts of an assembly which the bottom section may be part of. For example, the rest fluid may be drained through at least one fluid outlet that is connected to at least one sedimentation channel of an assembly which the bottom section may be part of. Preferably, the step of letting the solid components (e.g., cells) to be separated settle is a step of letting the solid components settle in the at least one sedimentation channel of the inclined plate settler that is part of the assembly in accordance with the present disclosure. In this embodiment, the rest fluid (i.e., the solid-depleted fluid) may be drained at the upper portion of the at least one sedimentation channel that is part of the plate settler in accordance with the present disclosure, e.g., through at least one fluid outlet that is connected to the at least one sedimentation channel.

According to some embodiments, the solid components to be separated are precipitates. These precipitates may form by chemical reactions in the fluid, and are preferably already present in solid form in the fluid when it is fed to the bottom section, but may also precipitate from the fluid, e.g., in the plate settler in accordance with the present disclosure.

In another embodiment of the separation of solid components from a fluid in accordance with the present disclosure, settled components are collected by pumping a wash fluid (e.g., a wash buffer) to at least one collection channel of the bottom section and by pumping the settled components and the wash fluid from at least one collection channel of the bottom section. Such collection may be performed at regular intervals. The frequency of collection (i.e., the intervals) should be adjusted depending, e.g., on the concentration of solid components in the fluid comprising the solid components. When the solid components are cells, also the tendency of these cells to adhere to surfaces should be taken into account when adjusting the frequency of collection. In a particularly preferred embodiment, the wash fluid should have an equal, preferably a higher density than the fluid comprising the solid components to be separated, and a lower density than the solid components. This is to ensure that the solid components can sediment into the wash fluid and to reduce mixing of the wash fluid with the fluid in accordance with the present disclosure. When the fluid comprising the solid components is a cell culture broth fluid and the solid components are cells, the wash fluid may comprise 14 g/L sodium chloride, 0.2 g/L potassium dihydrogen phosphate, 1.15 g/L sodium dihydrogen phosphate, and have a pH of 7.

When performing the separation of solid components from a fluid in accordance with the present disclosure, the inventors have found that solid components (e.g., cells) that are contained in a fluid (e.g., a cell culture broth fluid) can be efficiently separated from said fluid with minimal loss of any components that are dissolved in the fluid, such as proteins. Accordingly, according to some embodiments, the amount of solid components in the drained rest fluid is less than 20%, preferably less than 10%, most preferably less than 5% of the amount of solid components in the fluid that is fed to the at least one inlet channel of the bottom section. In another embodiment, the amount of a protein in the drained rest fluid is more than 80%, preferably more than 90%, most preferably more than 95% of the amount of said protein in the fluid that is fed to the at least one inlet channel of the bottom section. The amount of solid components in a fluid preferably refers to the concentration (e.g., in volume per volume) of solid components in said fluid. The skilled person will be aware of various methods to determine such concentration. For example, (relative) concentrations of solid components in a fluid can be determined by turbidity measurements. The amount of a protein in a fluid preferably refers to the concentration (e.g., in weight per volume or in activity units per volume) of the protein in said fluid. The skilled person will be aware of various methods to determine such concentration. For example, FVIII concentration in weight per volume can be determined by antigen ELISA. FVIII concentration in activity units per volume (i.e., FVIII activity) can be determined by chromogenic assays. Such chromogenic assays allow the determination of active FVIII, and yield the concentration, e.g., in international units (IU) per mL.

In another embodiment of the separation of solid components from a fluid in accordance with the present disclosure, the fluid comprising the solid components is continuously fed to the at least one inlet channel of the bottom section. In this embodiment, it is preferable that the rest fluid (i.e., the solid-depleted fluid) is also continuously drained. The skilled person will be aware of how to adjust the volumetric flow rate into the bottom section to ensure that the solid components have sufficient time to settle, e.g., in the at least one sedimentation channel in accordance with the present disclosure. When the method of the present disclosure is used to separate cells from fluid containing a protein (e.g., a biopharmaceutical drug), the continuous feed into the bottom section may be from a bioreactor comprising a continuous cell culture. Such continuous cell culture may be a chemostat, turbidostat or perfusion culture. Preferably, such continuous cell culture is a chemostat culture.

The temperature at which the separation of the present disclosure is performed is not particularly limited. The skilled person will be aware of how to select an appropriate temperature based on, e.g., the stability of any used materials and of any substances contained in the fluid comprising solid components. However, temperature differences within the assembly that is used for performing the separation of solid components in accordance with the present disclosure can result in temperature-induced density differences, which can lead to convection and thereby reduce the efficiency of separation between the wash fluid and the rest fluid. Therefore, it is preferable that the separation of solid components from a fluid in accordance with the present disclosure is performed at a uniform temperature, i.e., that the assembly (comprising, e.g., a bottom section and a plate settler) that is used for performing the method is kept at a set temperature +/−5° C., preferably at a set temperature +/−3° C.

Consistent with the above, the present inventors have found that cell removal from a cell culture broth fluid is particularly efficient when the assembly in accordance with the present disclosure is situated in a cold room with a temperature of between 2° C. and 8° C. Accordingly, according to some embodiments the separation in accordance with the present disclosure is performed at a temperature of between 0° C. and 10° C. (i.e., at a set temperature of 5° C.+/−5° C.), preferably at a temperature of between 2° C. and 8° C. (i.e., at a set temperature of 5° C.+/−3° C.). Such temperatures can be reached, e.g., by situating the assembly in a cold room. If, in the method in accordance with the present disclosure, the assembly is connected to a bioreactor, the bioreactor may be operated at a temperature that is different from the temperature at which the separation of solid components from a fluid is performed. In particular, if the separation in accordance with the present disclosure is performed at a temperature of between 0° C. and 10° C. or between 2° C. and 8° C. by situating the assembly in a cold room, the bioreactor is preferably operated at a higher temperature (e.g., 37° C.) and therefore not situated in the cold room.

Plate settlers and bottom sections in accordance with the embodiments described above are disclosed in PCT/EP2019/066009, which is hereby incorporated in its entirety. The plate settlers and bottom sections described in PCT/EP2019/066009 are preferred embodiments of the “plate settler for protein separation” and the “plate settler for cell separation” as well as the bottom sections that may be connected to these plate settlers in accordance with the present invention.

In the above description of plate settlers/bottom sections that may be used in the method for continuous recovering of a protein from a fluid in accordance with the present invention, when the solid component is precipitated protein, the plate settler is a “plate settler for protein separation” in accordance with the present invention. Thus, in a preferred embodiment of the method for continuous recovering of a protein from a fluid in accordance with the present invention, the “plate settler for protein separation” is an inclined plate settler with a lower portion, an upper portion, and at least one sedimentation channel for letting the precipitated protein settle, said sedimentation channel extend from the lower portion to the upper portion; the inclined plate settler being configured to be oriented during use such that the at least one sedimentation channel extends from the lower portion to the upper portion in a direction that is inclined with respect to the direction of gravity; wherein the at least one sedimentation channel is connected to a fluid outlet for draining a rest fluid at the upper portion.

For protein separation it is particularly preferable that the plate settler for protein separation comprises a relatively long sedimentation channel. Therefore, optionally, the length of the sedimentation channel is between 20 cm and 150 cm, preferably between 20 cm and 100 cm, more preferably between 20 cm and 80 cm, more preferably between 30 cm and 70 cm, more preferably between 40 cm and 60 cm, most preferably about 50 cm.

Preferably, in the above embodiment of the method for continuous recovering of a protein from a fluid in accordance with the present invention, the at least one sedimentation channel of the plate settler for protein separation is connected to a bottom section, wherein the bottom section comprises at least one inlet channel for feeding the fluid comprising the precipitated protein to the plate settler, and at least one collection channel for collecting the settled precipitated protein descending from the at least one sedimentation channel; wherein said at least one inlet channel and said at least one collection channel are fluidly separated from each other, said inlet channel and said collection channel being connected to said at least one sedimentation channel, to form fluid connections between said at least one inlet channel and said at least one sedimentation channel and between said at least one collection channel and said at least one sedimentation channel, respectively.

Preferably, in this embodiment of the method for continuous recovering of a protein from a fluid in accordance with the present invention, the bottom section that is connected to the plate settler for protein separation further comprises at least one wash fluid supply channel for supplying a wash fluid to one sedimentation channel or to one collection channel, said at least one wash fluid supply channel being fluidly separated from other wash fluid supply channels and from all inlet channels.

Optionally, in this embodiment of the method for continuous recovering of a protein from a fluid in accordance with the present invention, the at least one wash fluid supply channel and the at least one collection channel corresponding to the same sedimentation channel of the plate settler for protein separation are fluidly connected by an opening in a wall portion shared by said wash fluid supply channel and said collection channel.

Preferably, in the above embodiment of method for continuous recovering of a protein from a fluid in accordance with the present invention, the fluid comprising the precipitated protein is supplied to the bottom section (which is connected to the plate settler for protein separation) through the at least one inlet channel, and a wash fluid is supplied through the at least one wash fluid supply channel; wherein the density of the wash fluid is higher than the density of the fluid comprising the precipitated protein; and wherein the rest fluid is drained through the fluid outlet at the upper portion and the settled precipitated protein is drained through the collection channel.

Preferably, in the above embodiment of method for continuous recovering of a protein from a fluid in accordance with the present invention, the density of the wash fluid is between 0.3% and 1.5% higher than the density of the fluid comprising the precipitated protein, preferably between 0.55% and 1.20% higher than the density of the fluid comprising the precipitated protein.

As mentioned above, the higher density of the wash fluid compared to the density of the fluid comprising the precipitated protein is to increase the efficiency of the desired separation process. It may also lower or even avoid losses of wash fluid as the tendency of wash fluid accidentally being transported up the sedimentation channel (and possibly even being drained through a top end outlet) may be lowered. Thus, the higher density of the wash fluid compared to the density of the fluid comprising the precipitated protein is to ensure that the precipitated protein can sediment into the wash fluid and to reduce mixing of the wash fluid with the fluid in accordance with the present disclosure. Therefore, the density (and not the composition) of the wash fluid is decisive when choosing a wash fluid for the method of the present invention. Hence, in principle any solute can be used to adjust the density of the wash fluid. The skilled person will be well aware of suitable substances that can be added to the wash fluid in order to adjust its density.

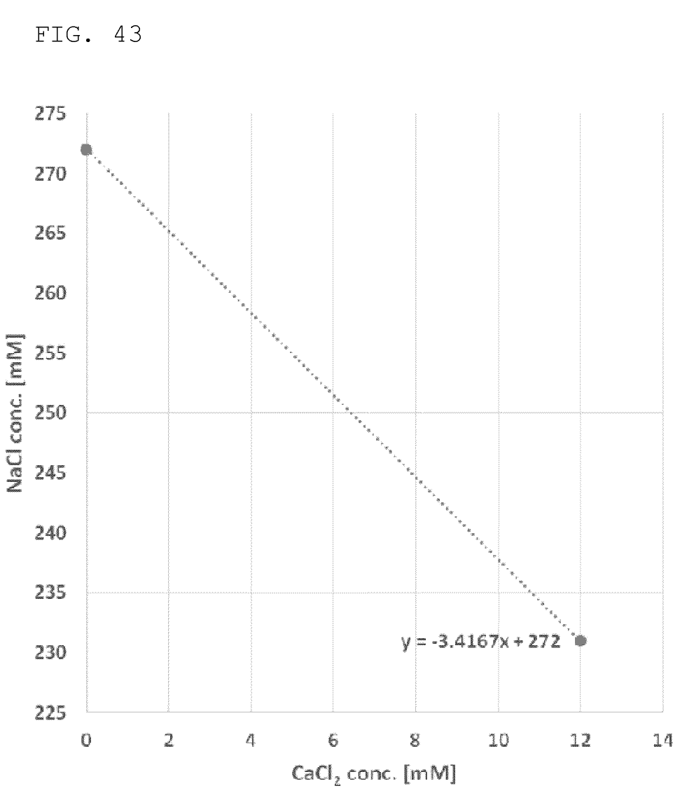

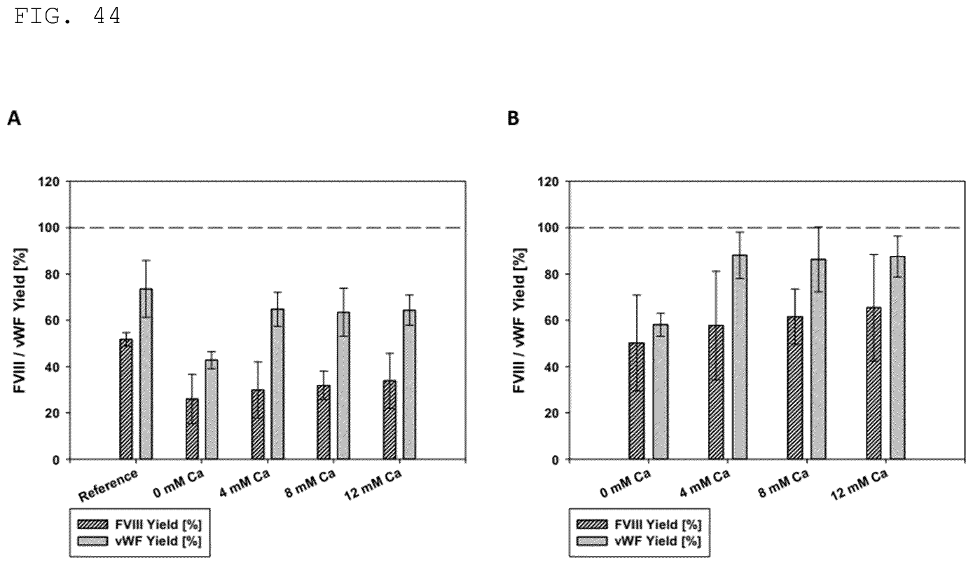

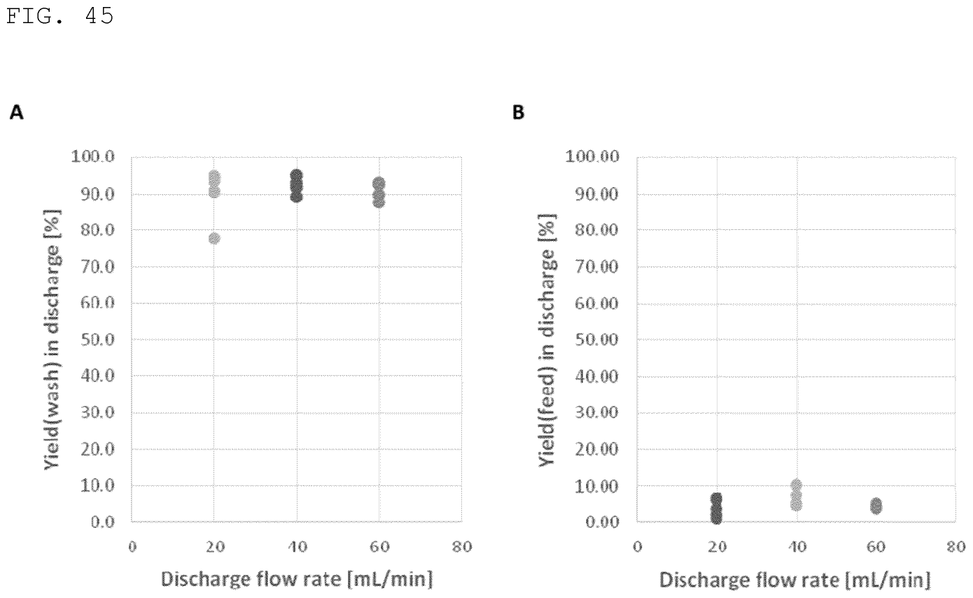

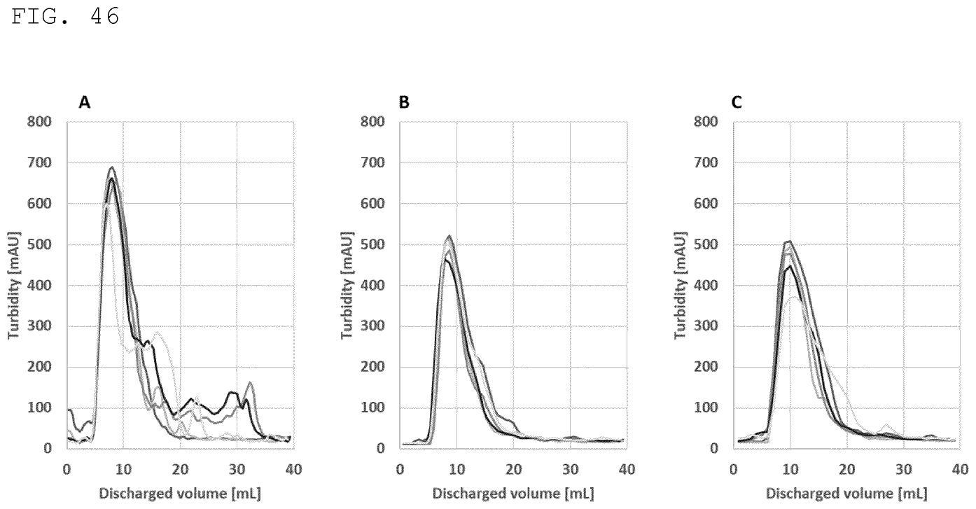

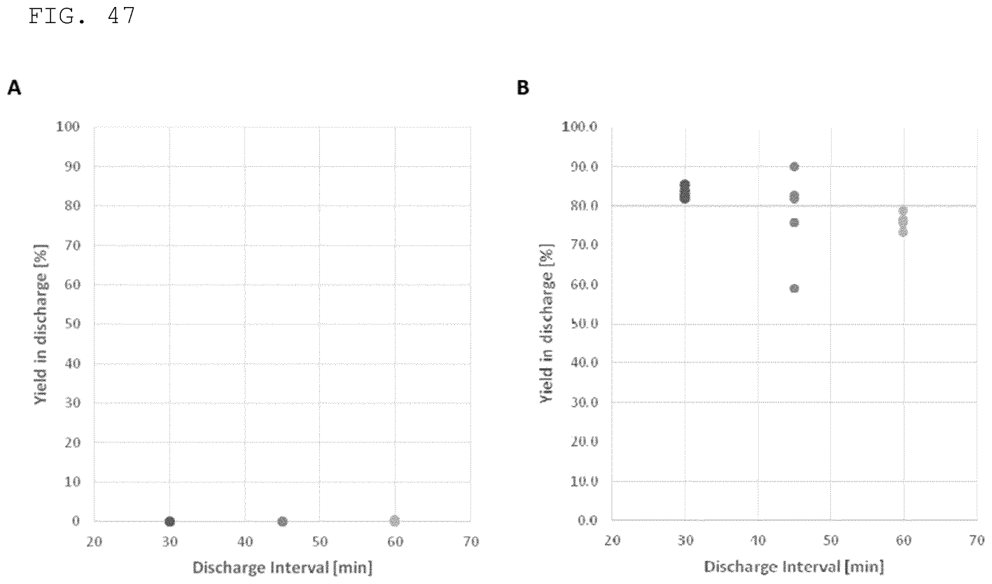

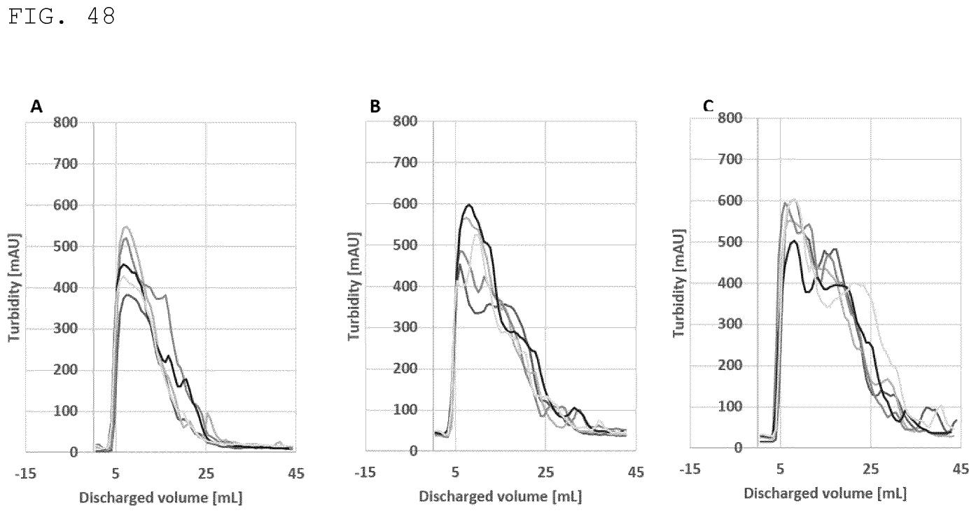

Exemplary wash fluids that are preferably used in the above embodiment of the method for continuous recovering of a protein from a fluid in accordance with the present invention, in particular when the fluid in accordance with the present invention is a cell culture medium, comprise Tris and sodium chloride. For example, the wash fluid may comprise Tris at a concentration of about 2 mM and sodium chloride at a concentration of about 272 mM. Preferably, the wash fluid may further comprise calcium chloride. In this embodiment, the wash fluid may comprise Tris at a concentration of about 2 mM, sodium chloride at a concentration of about 231 mM and calcium chloride at a concentration of about 12 mM. However, in this embodiment the concentrations of sodium chloride and calcium chloride may be varied, as long as the density of the wash fluid is kept equal. Further possible combinations of sodium chloride and calcium chloride concentrations that, in combination with 2 mM Tris, yield equal densities as the wash fluids comprising about 2 mM Tris and sodium chloride and/or calcium chloride at the concentrations indicated above are given in FIG. 43. Thus, as yet another alternative, the wash fluid in accordance with the present invention may comprise Tris at a concentration of about 2 mM, and comprise sodium chloride and/or calcium chloride at any (corresponding) concentrations derivable from FIG. 43. Accordingly, further exemplary wash fluids of this embodiment comprise about 2 mM Tris, about 4 mM calcium chloride and about 258 mM sodium chloride, or about 2 mM Tris, about 8 mM calcium chloride and about 245 mM sodium chloride. The pH of the wash fluid is chosen, e.g., with regard to the stability of the precipitate, and may be 7.5 or higher, preferably 8 or higher, most preferably about 8.25.

During continuous operation, it may be advantageous to let the precipitated protein settle for a while before draining it through the collection channel. Thus, in one embodiment of the method for continuous recovering of a protein from a fluid in accordance with the present invention, the wash fluid is supplied through the at least one wash fluid supply channel and the settled precipitated protein is drained through the collection channel at regular intervals. These regular intervals may be between 15 min and 45 min, but are preferably about 30 min. The volumetric flow rate of supplying the wash fluid through the at least one wash fluid supply channel and draining the settled precipitated protein through the collection channel may be about 20 to 60 mL/min, preferably about 40 mL/min.

In the above description of plate settlers/bottom sections that may be used in the method for continuous recovering of a protein from a fluid in accordance with the present invention, when the solid component is cells, the plate settler is a “plate settler for cell separation” in accordance with the present invention. Thus, in a preferred embodiment of the method for continuous recovering of a protein from a fluid in accordance with the present invention, the “plate settler for cell separation” is an inclined plate settler with a lower portion, an upper portion, and at least one sedimentation channel for letting the cells settle, said sedimentation channel extend from the lower portion to the upper portion; the inclined plate settler being configured to be oriented during use such that the at least one sedimentation channel extends from the lower portion to the upper portion in a direction that is inclined with respect to the direction of gravity; wherein the at least one sedimentation channel is connected to a fluid outlet for draining a rest fluid at the upper portion.

Preferably, in this embodiment of the method for continuous recovering of a protein from a fluid in accordance with the present invention, the at least one sedimentation channel of the plate settler for cell separation is connected to a bottom section, wherein the bottom section comprises at least one inlet channel for feeding the fluid comprising the cells and the protein to the plate settler, and at least one collection channel for collecting the settled cells descending from the at least one sedimentation channel; wherein said at least one inlet channel and said at least one collection channel are fluidly separated from each other, said inlet channel and said collection channel being connected to said at least one sedimentation channel, to form fluid connections between said at least one inlet channel and said at least one sedimentation channel and between said at least one collection channel and said at least one sedimentation channel, respectively.

Preferably, in this embodiment of the method for continuous recovering of a protein from a fluid in accordance with the present invention, the bottom section that is connected to the plate settler for cell separation further comprises at least one wash fluid supply channel for supplying a wash fluid to one sedimentation channel or to one collection channel, said at least one wash fluid supply channel being fluidly separated from other wash fluid supply channels and from all inlet channels.

Optionally, in this embodiment of the method for continuous recovering of a protein from a fluid in accordance with the present invention, the at least one wash fluid supply channel and the at least one collection channel corresponding to the same sedimentation channel of the plate settler for cell separation are fluidly connected by an opening in a wall portion shared by said wash fluid supply channel and said collection channel.

Preferably, in the above embodiment of method for continuous recovering of a protein from a fluid in accordance with the present invention, the fluid comprising the cells and the protein is supplied to the bottom section (which is connected to the plate settler for cell separation) through the at least one inlet channel, and a wash fluid is supplied through the at least one wash fluid supply channel; wherein the density of the wash fluid is higher than the density of the fluid comprising the cells and the protein; and wherein the settled cells are drained through the collection channel and the rest fluid comprising the protein is drained through the fluid outlet at the upper portion. The (rest) fluid comprising the protein is subsequently subjected to the further steps of the method for continuous recovering of a protein from a fluid in accordance with the present invention, e.g. to the protein precipitation step and the protein separation step.

As mentioned above, the higher density of the wash fluid compared to the density of the fluid comprising the cells and the protein is to increase the efficiency of the desired separation process. It may also lower or even avoid losses of wash fluid as the tendency of wash fluid accidentally being transported up the sedimentation channel (and possibly even being drained through a top end outlet) may be lowered. Thus, the higher density of the wash fluid compared to the density of the fluid comprising the cells and the protein is to ensure that the cells can sediment into the wash fluid and to reduce mixing of the wash fluid with the fluid in accordance with the present disclosure. Therefore, the density (and not the composition) of the wash fluid is decisive when choosing a wash fluid for the method of the present invention. Hence, in principle any solute can be used to adjust the density of the wash fluid. The skilled person will be well aware of suitable substances that can be added to the wash fluid in order to adjust its density.

During continuous operation, it may be advantageous to let the cells settle for a while before draining them through the collection channel. Thus, in one embodiment of the method in accordance with the present invention, the wash fluid is supplied through the at least one wash fluid supply channel and the settled cells are drained through the collection channel at regular intervals. These regular intervals may be between 5 min and 90 min, 15 min to 85 min, 25 min to 80 min, 35 min to 75 min, 45 min to 70 min, 55 min to 65 min, preferably about 60 min. The volumetric flow rate of supplying the wash fluid through the at least one wash fluid supply channel and draining the settled cells through the collection channel may be about 50 to 70 mL/min, preferably about 60 mL/min.

In one embodiment of the method for continuous recovering of a protein from a fluid in accordance with the present invention, the method further comprises a re-solubilization step of re-solubilizing the precipitated protein after the protein separation step. Preferably, in the re-solubilization step the precipitated protein is re-solubilized using citrate or EDTA. Although before the present invention EDTA had been excluded as a potential candidate for re-solubilization because its high complexing capability for calcium was assumed to be detrimental for protein (e.g., Factor VIII) activity, the present inventors have surprisingly found that EDTA does not significantly impact on protein (e.g., Factor VIII) activity, and is therefore suitable for re-solubilization in accordance with the present invention. Moreover, the present inventors have found that EDTA is more efficient in re-solubilizing calcium phosphate precipitates than citrate. Accordingly, in a particularly preferred embodiment of the present invention, the precipitated protein is re-solubilized using EDTA. Such re-solubilization may be performed using EDTA at a final concentration of between 10 mM and 50 mM, preferably of between 20 mM and 30 mM, most preferably of about 25 mM.

In one embodiment of the method for continuous recovering of a protein from a fluid in accordance with the present invention, the re-solubilization step is performed at a temperature of between 0° C. and 8° C., preferably of between 2° C. and 8° C.

Biopharmaceutical drugs are of increasing commercial importance. Many biopharmaceutical drugs are proteins. These protein biopharmaceutical drugs are often produced in fluids, and thus need to be recovered before they can be formulated as pharmaceutical compositions. Thus, in a preferred embodiment of the method for continuous recovering of a protein from a fluid in accordance with the present invention, the protein to be recovered is a biopharmaceutical drug. Such biopharmaceutical drugs in accordance with the invention are not particularly limited, as long as the biopharmaceutical drugs are proteins. The biopharmaceutical drugs in accordance with the invention include both recombinant biopharmaceutical drugs and biopharmaceutical drugs from other sources such as biopharmaceutical drugs obtained from (human) plasma, but preferably the biopharmaceutical drugs in accordance with the invention are recombinant biopharmaceutical drugs. Biopharmaceutical drugs in accordance with the invention include, without limitation, blood factors, immunoglobulins, replacement enzymes, growth factors and their receptors, and hormones. Preferred blood factors include factor I (fibrinogen), factor II (prothrombin), tissue factor, factor V, factor VII and factor Vila, factor VIII, factor IX, factor X, factor XI, factor XII, factor XIII, von Willebrand Factor (VWF), prekallikrein, high-molecular-weight kininogen (HMWK), fibronectin, antithrombin III, heparin cofactor II, protein C, protein S, protein Z, plasminogen, alpha 2-antiplasmin, tissue plasminogen activator (tPA), urokinase, plasminogen activator inhibitor-1 (PAI1), and plasminogen activator inhibitor-2 (PAI2). Preferred immunoglobulins include immunoglobulins from human plasma, monoclonal antibodies and recombinant antibodies. The biopharmaceutical drugs in accordance with the present invention may include functional polypeptide variants. The biopharmaceutical drugs in accordance with the invention are preferably the respective human or recombinant human proteins (or functional variants thereof).

After recovering the biopharmaceutical drug of the present invention by the method for continuous recovering of a protein from a fluid in accordance with the present invention, the biopharmaceutical drug can be formulated into a pharmaceutical composition. Thus, the present invention also relates to a method for producing a pharmaceutical composition, comprising performing the method for continuous recovering of a protein from a fluid in accordance with the present invention, and subsequently formulating the recovered biopharmaceutical drug as a pharmaceutical composition. Such pharmaceutical composition can be prepared in accordance with known standards for the preparation of pharmaceutical compositions. For example, the composition can be prepared in a way that it can be stored and administered appropriately, e.g. by using pharmaceutically acceptable components such as carriers, excipients or stabilizers. Such pharmaceutically acceptable components are not toxic in the amounts used when administering the pharmaceutical composition to a patient.

The present invention provides a method for continuous recovering of a protein from a fluid, wherein the protein can be a biopharmaceutical drug, as well as a method for producing a pharmaceutical composition. Accordingly, the present invention is also directed to a recovered protein that is obtainable by the method for continuous recovering of a protein from a fluid in accordance with the present invention, including a biopharmaceutical drug that is obtainable by the method for continuous recovering of a protein from a fluid in accordance with the present invention, and the present invention is also directed to a pharmaceutical composition that is obtainable by the method for producing a pharmaceutical composition in accordance with the present invention.

In the above-described method for continuous recovering of a protein from a fluid, it is particularly advantageous when the plate settler comprises at least one sedimentation channel for letting the precipitated protein settle, which is relatively long. Accordingly, the present invention also provides a plate settler comprising a sedimentation channel of that length. Thus, the present invention is also directed to an inclined plate settler for separating a solid component (e.g., a precipitated protein, preferably a precipitated protein complex comprising Factor VIII and von Willebrand factor) from a fluid, wherein the plate settler comprises a lower portion, an upper portion, and at least one sedimentation channel for letting the solid component (e.g., the precipitated protein, preferably the precipitated protein complex comprising Factor VIII and von Willebrand factor) settle, said sedimentation channel extend from the lower portion to the upper portion; the plate settler being configured to be oriented during use such that the at least one sedimentation channel extends from the lower portion to the upper portion in a direction that is inclined with respect to the direction of gravity; wherein the at least one sedimentation channel is connected to a fluid outlet for draining a rest fluid at the upper portion and connected to a bottom section at the lower portion; wherein the bottom section comprises at least one inlet channel for feeding a fluid comprising the solid component to be separated to the plate settler, and at least one collection channel for collecting a settled component descending from the at least one sedimentation channel; wherein said at least one inlet channel and said at least one collection channel are fluidly separated from each other, said inlet channel and said collection channel being connected to said at least one sedimentation channel, to form fluid connections between said at least one inlet channel and said at least one sedimentation channel and between said at least one collection channel and said at least one sedimentation channel, respectively; wherein the bottom section further comprises at least one wash fluid supply channel for supplying a wash fluid to one sedimentation channel or to one collection channel, said at least one wash fluid supply channel being fluidly separated from other wash fluid supply channels and from all inlet channels; and wherein the length of the sedimentation channel is between 20 cm and 150 cm, preferably between 20 cm and 100 cm, more preferably between 20 cm and 80 cm, more preferably between 30 cm and 70 cm, more preferably between 40 cm and 60 cm, most preferably about 50 cm. In a preferred embodiment, the inclined plate settler contains a precipitated protein complex comprising Factor VIII and von Willebrand factor.

In the following, the present invention will be illustrated by examples, without being limited thereto.

### EXAMPLES

### Examples 1 to 6: Overview

In the presented examples 1 to 6, embodiments of the bottom section in accordance with the present disclosure (and, more generally, embodiments of the assembly in accordance with the present disclosure) were applied for separation of animal cells from an animal cell culture suspension and for separation of a precipitated solid from its fluid phase.

In examples 1 to 3, Chinese hamster ovarian (CHO) cells expressing a recombinant blood coagulation factor VIII (FVIII) were cultured continuously, wherein the CHO cell culture operation temperature was 37° C. On average, the cell culture broth exhibited a starting turbidity of 46.6 FNU. The bioreactor outlet was directly connected to the inlet of the bottom section in the assembly with the inclined plate settler that is schematically represented in FIG. 2. In these examples, the inclined plate settler was inclined by an angle α′=30° with respect to the vertical direction, being perpendicular to the horizontal direction (the direction of gravity). The angle with respect to the horizontal direction was thus 60°. The inclined plate settler was made from stainless steel with surfaces in contact with process fluid being electro polished to Ra<0.6 μm. The internal hold-up volume of the assembly was 803 mL. The settling section was separated into four sedimentation channels, i.e., settling plates (analogous to (21) in FIG. 2), which were separated by separating walls made ((25) in FIG. 2) of stainless steel in examples 1 and 2 and from PMMA in example 3. A wash solution was supplied to and used with the bottom section. The wash solution consisted of 14 g/L sodium chloride, 0.2 g/L potassium dihydrogen phosphate, 1.15 g/L sodium dihydrogen phosphate, pH 7.

The cell culture broth was continuously transported from the bioreactor to the assembly. The clarified fluid, i.e., cell depleted fluid, was continuously collected from the top outlet of the assembly. The separated solids were collected from the collection channels of the bottom section at regular intervals of 60 min. Collection of the separated solids from the solid collection channels of the bottom section was performed by simultaneous action of the wash fluid pump and the collected solids pump at a volumetric flow rate of 62 and 60 mL/min, respectively. The interval for cell collection, or solid collection in general, was optimized depending on the cell count, i.e., solid load, of the cell culture broth. The flow rate for cell collection or solid collection in general, was optimized depending on the characteristics of the solids, which for example could be a tendency of cells to adhere to surfaces, in order to prevent stalling of sedimented solids within the collection channels of the bottom section.

Samples for analysis were taken in regular intervals from the bioreactor and the fluid streams leaving the assembly. Glucose concentration in the fluid phase was determined using a commercial glucose analyzer (stat profile prime device, nova biomedical). Product (FVIII) concentration was determined by a chromogenic assay using the Chromogenix Coatest® SP4 Factor VIII kit. The chromogenic assay allows measurement of the FVIII co-factor activity, wherein it activates factor X to factor Xa together with factor IXa in the presence of phospholipids and calcium. The activated FXa hydrolyses the chromogenic substrate (S-2765), thus releasing the chromogenic group pNA, whose absorbance can be measured at 405 nm. Under the conditions of the assay factor X activation, and thus generation of the chromogenic substance, pNA is dependent on FVIII amount only (cf. Peyvandi, F., Oldenburg, J. & Friedman, K. D.: A critical appraisal of one-stage and chromogenic assays of factor VIII activity; Journal of thrombosis and haemostasis: JTH 14, 248-261 (2016)). The concentration of the analytes, glucose and FVIII, in the streams collected at the top and from the bottom section of the assembly was used to set up a mass balance, where the amount of analyte recovered in a given period was related to the amount produced/present in the bioreactor in the same period. Cell removal was evaluated by turbidity measurement using a Hach 2100Q, which is a portable turbidometer. The turbidometer measures light scattered by a sample in a round cuvette (25 mm diameter, 60 mm height) at an angle of 90 degrees relative to the direction of the incident light, where the light source is a light emitting diode.

### Example 1 for “BOTTOM SECTION FOR BEING CONNECTED TO AN ASSEMBLY WITH PLATE SETTLER, AND ASSEMBLY WITH PLATE SETTLER” (CHO Cell Separation with an Additional Fluid Circuit)

The inclined plate settler was cooled by a double jacket connected to a cryostat, which was set to 4° C. The double jacket and the cryostat are schematically indicated by the dashed lines with the pump in FIG. 14. The bottom section was not cooled. The single-use bag containing the wash fluid was placed in wet ice for temperature control, thus resulting in a temperature of approx. 0° C. Two runs, which lasted for 49 and 90 hours, respectively, were performed with this mode of temperature control.

In order to show that the bottom section of the inclined plate settler in accordance with the present disclosure allows to separate cells from the product containing liquid fraction with minimal product loss, glucose and FVIII concentration were measured. In the bottom section, cells were sedimented into the provided wash fluid, while the entire liquid fraction of the culture broth was collected at the top outlet. The wash buffer must have a density higher than the liquid fraction of the culture broth and a density lower than the solids. Thereby, cells can sediment into the wash buffer and minimal mixing of the wash fluid with the culture broth fluid is achieved. In the presented examples, this was the case for the specified wash buffer. Cells could be successfully removed while the product containing fluid fraction could be collected with high yield at the top outlet. The data for FVIII and glucose yield, are plotted in FIG. 15 and FIG. 16, with the values in Table 1 and Table 2. Turbidity as a measure for cell removal can be found in Table 1. Under the conditions in example 1, it is possible to use glucose as an indicator for product (FVIII), because it is not metabolized by the cells.

TABLE 1
Product (FVIII) yield given in percent of amount present
in the fluid fraction collected at the bottom and at the top
outlet of the assembly in example 1 and turbidity given in
FNU measured in the fluid collected at the top outlet in
example 1. The turbidity of the cell containing culture
broth was 46.6 FNU in average.

| Run 1 | | | | Run 2 | | | |
|---|---|---|---|---|---|---|---|
| | FVIII | | | | FVIII | | |
| Run | Yield | FVIII | Tur- | Run | Yield | FVIII | Tur- |
| dur- | at | Yield | bidity | dur- | at | Yield | bidity |
| ation | bottom | at top | at top | ation | bottom | at top | at top |
| [h] | outlet | outlet | outlet | [h] | outlet | outlet | outlet |
| 3 | 3.47 | 85.2 | 0.86 | 19 | 6.97 | 99.5 | 6.85 |
| 5 | 3.51 | 97.0 | 0.87 | 20 | below | 94.7 | 1.98 |
| | | | | | LOD | | |
| 6 | 3.04 | 94.0 | 0.77 | 21 | 4.62 | 93.0 | 1.03 |
| 8 | 2.48 | 97.4 | 0.95 | 24 | 5.86 | 94.7 | 1.81 |
| 24 | 3.84 | 97.8 | 1.24 | 27 | 5.21 | 93.0 | 2.00 |
| 25 | 3.93 | 97.8 | 0.95 | 40 | 5.24 | 92.6 | 4.87 |
| 29 | 3.76 | 106 | 1.27 | 44 | 5.19 | 92.8 | 1.87 |
| 31 | 2.76 | 98.6 | 1.32 | 49 | 5.30 | 92.8 | 2.71 |
| 47 | 2.79 | 99.1 | 2.06 | 65 | 5.35 | 89.6 | 2.58 |
| 48 | 3.01 | 97.0 | 2.47 | 68 | 5.33 | 91.5 | 4.08 |
| 49 | 3.12 | 96.5 | 2.16 | 72 | 5.65 | 91.4 | 3.23 |
| | | | | 89 | 5.74 | 92.5 | 8.42 |
| | | | | 90 | below | 90.7 | 7.83 |
| | | | | | LOD | | |

LOD = limit of detection; 0.2.

TABLE 2
Glucose yield given in percent of amount present
in the fluid fraction collected at the bottom and
at the top outlet of the assembly in example 1.

| Run 1 | | | Run 2 | | |
|---|---|---|---|---|---|
| | Glucose | Glucose | | Glucose | Glucose |
| | Yield | Yield | | Yield | Yield |
| Run duration | at bottom | at top | Run duration | at bottom | at top |
| [h] | outlet | outlet | [h] | outlet | outlet |
| 3 | 7.05 | 90.5 | 19 | 8.00 | 92.1 |
| 5 | 4.71 | 95.4 | 20 | 6.98 | 97.7 |
| 6 | 4.89 | 96.4 | 21 | 7.15 | 91.5 |
| 8 | 4.81 | 94.9 | 24 | 6.89 | 93.8 |
| 24 | 4.40 | 93.9 | 27 | 6.24 | 90.4 |
| 25 | 4.94 | 92.5 | 40 | 6.69 | 87.0 |
| 29 | 4.71 | 92.5 | 44 | 6.59 | 93.8 |
| 31 | 4.49 | 92.0 | 49 | 15.9 | 89.8 |
| 47 | 4.25 | 90.5 | 65 | 6.18 | 97.2 |
| 48 | 4.51 | 90.0 | 68 | n.d. | 88.7 |
| 49 | 4.65 | 90.5 | 89 | n.d. | 96.0 |

n.d. = not determined

### Example 2 for “BOTTOM SECTION FOR BEING CONNECTED TO AN ASSEMBLY WITH PLATE SETTLER, AND ASSEMBLY WITH PLATE SETTLER” (CHO Cell Separation without Additional Fluid Circuit)

In example 2, the assembly of the inclined plate settler with the bottom section, including all supplying and receiving vessels (except the bioreactor), was set up in a cold room, where the temperature was 2 to 8° C. The setup is schematically depicted in FIG. 17. The inclined plate settler and bottom section were identical to example 1. One run was performed under these conditions which lasted for 70 hours. In order to show that the bottom section of the inclined plate settler in accordance with the present disclosure allows to separate cells from the product containing liquid fraction with minimal product loss, glucose and FVIII concentration were measured. In the bottom section cells were sedimented into the provided wash fluid, while the entire liquid fraction of the culture broth was collected at the top outlet. The wash buffer must have a density higher than the liquid fraction of the culture broth and a lower density than the solids. Thereby, cells can sediment into the wash buffer and minimal mixing of the wash fluid with the culture broth fluid is achieved. In the presented examples, this was the case for the specified wash buffer. Cells could be successfully removed while the product containing fluid fraction could be collected with high yield at the top outlet. The data obtained in example 2 for FVIII and glucose yield are plotted in FIG. 18, with the values for product (FVIII) yield in Table 3 and values for glucose yield and turbidity measured in the samples collected at the top outlet as a measure for cell removal in Table 4. The turbidity data indicated cell removal was more efficient and more stable over time, when the inclined plate settler and bottom section were set up in the cold room as compared to cooling via the double jacket (as described in example 1).

TABLE 3
Product (FVIII) yield given in percent of amount present
in the fluid fraction collected at the bottom and
at the top outlet of the assembly in example 2.

| Run duration | FVIII Yield at | FVIII Yield at |
|---|---|---|
| [h] | bottom outlet | top outlet |
| 26 | 2.01 | 84.1 |
| 51 | 0.56 | 90.4 |
| 70 | 0.56 | 97.8 |

TABLE 4
Glucose yield given in percent of amount present in the fluid
fraction collected at the bottom and at the top outlet of the
assembly in example 2 and turbidity given in FNU measured in
the fluid collected at the top outlet in example 2. The turbidity
of the cell containing culture broth was 46.6 FNU in average.

| Run duration | Glucose Yield at | Glucose Yield at | Turbidity at |
|---|---|---|---|
| [h] | bottom outlet | top outlet | top outlet |
| 18 | 2.67 | 99.5 | 2.62 |
| 22 | 2.67 | 101 | 0.72 |
| 26 | 2.84 | 99.0 | 0.87 |
| 42 | 2.67 | 101 | 1.38 |
| 47 | 2.58 | 100 | 1.98 |
| 51 | 2.67 | 93.8 | 1.69 |
| 67 | 2.49 | 94.7 | 1.49 |
| 70 | 2.31 | 95.2 | 1.06 |

### Example 3 for “BOTTOM SECTION FOR BEING CONNECTED TO AN ASSEMBLY WITH PLATE SETTLER, AND ASSEMBLY WITH PLATE SETTLER” (CHO Cell Separation with PMMA Physical Barriers)

In example 3, the assembly of the inclined plate settler with the bottom section, including all supplying and receiving vessels (except the bioreactor), was set up in a cold room, where the temperature was 2° C. to 8° C. The setup is schematically depicted in FIG. 17. The inclined plate settler was made of stainless steel with surfaces in contact with cell culture broth being electro polished to Ra<0.6 μm. The settling section was separated into four sedimentation channels, i.e. settling plates (analogous to (21) in FIG. 2), which were separated by separating walls made of polymethylmethacrylat (PMMA) ((25) in FIG. 2). One run was performed with this setup, which lasted for 94 hours. In order to show that the bottom section of the inclined plate settler in accordance with the present disclosure allows to separate cells from the product containing liquid fraction with minimal product loss, glucose and FVIII concentration were measured. In the bottom section, cells were sedimented into the provided wash fluid, while the entire liquid fraction of the culture broth was collected at the top outlet. The wash buffer must have a density higher than the liquid fraction of the culture broth and a lower density than the solids. Thereby, cells can sediment into the wash buffer and minimal mixing of the wash fluid with the culture broth fluid is achieved. In the presented examples, this was the case for the specified wash buffer. Cells could be successfully removed while the product containing fluid fraction could be collected with high yield at the top outlet. The data for FVIII and glucose yield are plotted in FIG. 19, with the values for product (FVIII) yield in Table 5 and values for glucose yield and turbidity measured in the samples collected at the top outlet as a measure for cell removal in Table 6. The turbidity data indicate cell removal was more efficient and more stable over time, when the inclined plate settler and bottom section were set up in the cold room as compared to cooling via the double jacket (as described in example 1). There was no difference in separation performance (based on the available data) with regard to the material of the separating walls between example 2 (stainless steel) and example 3 (PMMA).

TABLE 5
Product (FVIII) yield given in percent of amount present
in the fluid fraction collected at the bottom and
at the top outlet of the assembly in example 3.

| Run duration | FVIII Yield at | FVIII Yield at |
|---|---|---|
| [h] | bottom outlet | top outlet |
| 6 | 2.43 | 95.2 |
| 29 | 1.18 | 99.9 |
| 54 | 1.18 | 93.9 |
| 78 | below LOD | 96.1 |
| 94 | below LOD | 95.8 |

LOD = limit of detection; 0.2. IU/ml.

TABLE 6
Glucose yield given in percent of amount present in the fluid
fraction collected at the bottom and at the top outlet of
the assembly in example 3 and turbidity given in FNU measured
in the fluid collected at the top outlet in example 3.

| Run duration | Glucose Yield at | Glucose Yield at | Turbidity at |
|---|---|---|---|
| [h] | bottom outlet | top outlet | top outlet |
| 6 | 2.77 | 105 | 1.27 |
| 21 | 2.59 | 102 | 0.93 |
| 25 | 2.68 | 104 | 1.12 |
| 29 | 2.50 | 101 | 0.83 |
| 45 | 2.59 | 100 | 0.92 |
| 49 | 3.93 | 98.6 | 0.92 |
| 54 | 2.77 | 94.3 | 1.54 |
| 70 | 2.50 | 97.2 | 0.82 |
| 74 | 2.50 | 100 | 1.23 |
| 78 | 2.06 | 98.1 | 1.2 |
| 94 | 2.50 | 95.7 | 0.92 |

### Example 4 for “BOTTOM SECTION FOR BEING CONNECTED TO AN ASSEMBLY WITH PLATE SETTLER, AND ASSEMBLY WITH PLATE SETTLER” (Supply and Collection of Process Streams to the Bottom Section for Cleaning in Place)

Example 4 relates to an embodiment of the assembly of the bottom section with an inclined plate settler including switchable connections to supplying and receiving vessels. The inclined plate settler and bottom section with the connected vessels were assembled as a “closed system”. The used vessels were multi-use glassware that was autoclaved prior to use. The connecting elements were made from silicone and c-flex tubing, Luer and metal connectors. Silicone tubing and Luer connectors were considered as single-use. However, all vessels and connecting elements could be also be (1) single use and (2) pre-assembled. In the default-state the three-way-valves situated at the bottom section were configured such that a direct fluid connection between vessels [1], [2] and [4] and the assembly was made. For cleaning in place (CIP) 1 M sodium hydroxide solution was pumped from a supplying vessel ([1] in FIG. 20) into the assembly of plate settler and bottom section. The assembly was completely filled and the sampling valves (marked with +) flushed with 1 M sodium hydroxide. The assembly was incubated for at least 15 minutes with 1 M sodium hydroxide. After the incubation time, the three-way-valves situated at the bottom section were switched such that a direct fluid connection between the assembly and a receiving vessel ([3] in FIG. 20) was established. The 1 M sodium hydroxide solution was drained to the receiving vessel by gravity flow. During draining of fluid from the assembly, an inflow of air was provided via receiving vessel [6]. When the assembly was empty, the three-way-valves were switched back to the original position creating a direct fluid connection between vessels [1], [2] and [4] and the assembly and could be filled anew. The filling and draining procedure including the flush of the sampling valves was repeated at least twice with an aqueous buffer solution (e.g. 8 g/L sodium chloride, 0.2 g/L potassium dihydrogen phosphate, 1.15 g/L sodium dihydrogen phosphate, pH 7). Completeness of the CIP procedure was confirmed by pH measurement of samples taken from the sampling valves, where a pH of <7.2 was accepted.

### Example 5 for “BOTTOM SECTION FOR BEING CONNECTED TO AN ASSEMBLY WITH PLATE SETTLER, AND ASSEMBLY WITH PLATE SETTLER” (Separation of a Precipitated Solid at Various Collection Flow Rates in the Presence of an Amino Acid)

In example 5, a precipitate suspension was separated into its solid fraction, i.e. the precipitate, and its fluid fraction, i.e. the precipitation supernatant. The precipitate suspension was produced by supplementation of an aqueous solution comprised of 10 mM Tris(hydroxymethyl)-aminomethan, 100 mM sodium chloride and 100 mg/mL Tryptophan pH 8.5 with 2.7 mM phosphate ions and 15 mM calcium ions. The formed solid phase was non-stoichiometric calcium phosphate. The precipitate suspension was directly and continuously transported to the inlet of the bottom section in assembly with the inclined plate settler. In these examples, the inclined plate settler was inclined at by an angle α′=30° from the vertical direction, i.e., an angle of α=60° with respect to the horizontal direction (the direction of gravity). The inclined plate settler was made from stainless steel where the surfaces in contact with process fluid were electro polished to Ra<0.6 μm. The internal hold-up volume consisting of bottom section and an inclined plate settler with a single settling channel was 630 mL. A wash solution was supplied to and used with the bottom section. The wash fluid was an aqueous solution containing 2 mM Tris(hydroxymethyl)-aminomethan, 252 mM sodium chloride and 6 mM calcium chloride. The wash fluid density must be higher than the density of the fluid in the precipitate suspension and lower than the density of the suspended solids in order for the solids to settle from the fluid they were originally suspended in into the wash buffer provided in the bottom section. For the precipitate suspension and the wash fluid in this example, the densities were matching this criterion.

During operation of the assembly, the solid depleted fluid was continuously collected from the top outlet of the assembly. Separated solids were collected from the collection channels of the bottom section at regular timely intervals of 15 min. Solid collection was achieved by simultaneous action of the wash fluid and the solid collection pump at volumetric flow rates of 20, 40 and 60 mL/min.

In order to demonstrate successful separation and wash of the suspended solid (i.e., the precipitate), a tracer, namely Tryptophan, was supplemented to the precipitate suspension. Carry over of fluid parts originally comprised in the precipitate suspension to the wash fluid and thus the collected solids could be monitored via absorbance measurement based on the absorbance maximum at 280 nm of Tryptophan. Samples to be measured were taken after every solid collection cycle from the fluid streams leaving the assembly. The data plotted in FIG. 21 (see also Table 7) show low yield of Tryptophan in the collected solids suspended in the wash solution over the entire range of collection flow rates tested. Low Tryptophan yield in the wash fluid corresponds to low carry over from the solid bearing fluid to be separated. Consequently, the largest fraction of fluid present in the collected solids fraction was wash buffer, which demonstrates efficient precipitate wash.

TABLE 7
Yield values of Tryptophan in the fraction containing the collected
solids (i.e. the precipitate) suspended in wash fluid obtained at
varying collection flow rates. Tryptophan was originally comprised
in the precipitate suspension. The volume of the discharge fraction
was 40 mL independent of the discharge volumetric flow rate.

| | | Yield of amino |
|---|---|---|
| Number of discharge | | acid in the wash |
| cycle at volumetric | | solution bearing the |
| flow rate | Volumetric flow | collected solids |
| [—] | [mL/min] | [%] |
| 1 | 20 | 1.02 |
| 2 | 20 | 2.25 |
| 3 | 20 | 3.91 |
| 4 | 20 | 6.45 |
| 5 | 20 | 6.47 |
| 1 | 40 | 10.14 |
| 2 | 40 | 7.43 |
| 3 | 40 | 5.34 |
| 4 | 40 | 4.65 |
| 5 | 40 | 5.15 |
| 1 | 60 | 5.22 |
| 2 | 60 | 4.30 |
| 3 | 60 | 3.78 |
| 4 | 60 | 4.25 |
| 5 | 60 | 4.07 |

### Example 6 for “BOTTOM SECTION FOR BEING CONNECTED TO AN ASSEMBLY WITH PLATE SETTLER, AND ASSEMBLY WITH PLATE SETTLER” (Separation of a Precipitate at Various Collection Flow Rates in the Presence of a Colorant)

In example 6, a precipitate suspension was separated into its solid fraction, i.e. the precipitate, and its fluid fraction, i.e. the precipitation supernatant. The precipitate suspension was produced by supplementation of an aqueous solution comprising 10 mM Tris(hydroxymethyl)-aminomethan and 100 mM sodium chloride pH 8.5 with 2.7 mM phosphate ions and 15 mM calcium ions. The precipitate suspension was directly and continuously transported to the inlet of the bottom section in assembly with the inclined plate settler. In these examples, the inclined plate settler was inclined at by an angle α′=30° from vertical. The inclined plate settler was made from stainless steel where the surfaces in contact with process fluid were electro polished to Ra<0.6 μm. The internal hold-up volume consisting of bottom section and an inclined plate settler with a single settling channel was 630 mL. A wash solution was supplied to and used with the bottom section. The wash fluid was an aqueous solution containing 2 mM Tris(hydroxymethyl)-aminomethan, 252 mM sodium chloride, 6 mM calcium chloride and 25 mg/L Patent Blue V, which has an absorbance maximum at 620 nm. The wash fluid density must be higher than the density of the fluid in the precipitate suspension and lower than the density of the suspended solids in order for the solids to settle from the fluid they were originally suspended in into the wash buffer provided in the bottom section. For the precipitate suspension and the wash fluid in this example, the densities were matching this criterion.

During operation of the assembly, the solid depleted fluid was continuously collected from the top outlet of the assembly. Separated solids, were collected from the collection channels of the bottom section at regular timely intervals of 15 min. Solid collection was achieved by simultaneous action of the wash fluid and the solid collection pump at volumetric flow rates of 20, 40 and 60 mL/min.

In order to demonstrate successful separation and wash of the suspended solid (i.e., the precipitate), a tracer, namely Patent Blue V, was supplemented to the wash fluid. Carry over of fluid parts originally comprised in the precipitate suspension to the wash fluid and thus the collected solids could be monitored via absorbance measurement based on the absorbance maximum at 620 nm of Patent Blue V. Samples for analysis were taken after every solid collection cycle from the fluid streams leaving the assembly. The data plotted in FIG. 22 (see also Table 8) show high yield of Patent Blue V in the collected solids suspended in wash fluid. Here, low yield corresponds to high carry over from the solid bearing fluid to be separated. Therefore, the high yield values support successful separation of precipitate from the precipitate suspension with efficient wash of the collected precipitate.

TABLE 8
Yield values of Patent Blue V collected solids suspended in wash fluid
obtained at varying collection flow rates. Patent Blue V was originally
comprised in the wash fluid. The volume of the discharge fraction
was 40 mL independent of the discharge volumetric flow rate.

| Number of discharge | | Yield of colorant |
|---|---|---|
| cycle at volumetric | | in the wash solution |
| flow rate | Volumetric flow | bearing the collected |
| [—] | [mL/min] | solids [%] |
| 1 | 20 | 77.7 |
| 2 | 20 | 90.6 |
| 3 | 20 | 94.2 |
| 4 | 20 | 93.4 |
| 5 | 20 | 94.8 |
| 1 | 40 | 89.2 |
| 2 | 40 | 91.8 |
| 3 | 40 | 94.7 |
| 4 | 40 | 95.1 |
| 5 | 40 | 92.9 |
| 1 | 60 | 87.3 |
| 2 | 60 | 92.9 |
| 3 | 60 | 92.4 |
| 4 | 60 | 92.7 |
| 5 | 60 | 89.6 |

It will be apparent to those skilled in the art that various modifications and variations can be made in the disclosed devices and systems without departing from the scope of the disclosure. Other aspects of the disclosure will be apparent to those skilled in the art from consideration of the specification and practice of the features disclosed herein. It is intended that the specification and examples be considered as exemplary only. Many additional variations and modifications are possible and are understood to fall within the framework of the disclosure.

### Examples 7 to 10: Overview

The following materials and methods were for used for Examples 7 to 10:

### Materials

### Cell Culture Supernatant

Clarified Chinese hamster ovary (CHO) cell culture supernatant from a CHO cell line secreting rFVIII (octocog alfa) and rVWF (vonicog alfa) was provided by Baxalta Innovations GmbH (Orth/Donau, Austria). Removal of cells and clarification was achieved by a combination of depth and membrane filtration. The cell culture supernatant was stored at <−60° C. Whenever needed aliquots were thawed overnight at 2-8° C. in a water bath.

### Chemicals

Albumin from human serum, acetic acid glacial, BIS-TRIS, CaCl₂*2H2O, calcium colorimetric kit, HEPES, rabbit serum, trisodium citrate, Tween80, and 4-Hydroxy-4methyl-2-pentanone were bought from Sigma Aldrich (St. Louis, Mo., USA). DPBS and supplies for gel electrophoresis were from Thermo Fisher Scientific (Waltham, Mass., USA). Analytical grade NaOH, Citrate monohydrate, dichloromethane, Na₂HPO₄, NaCl, PEG10,000, PEG20,000, Spectroquant® phosphate test, Tris(hydroxymethyl)-aminomethan and Titriplex®III were from Merck (Darmstadt, Germany). PEG2,000, 4,000, 6,000 and 8,000 were obtained from Fluka Chemie GmbH. The Chromogenix Coatest SP4 VIII kit was purchased from Coachrom Diagnostica GmbH (Maria Enzersdorf, Austria). The VWF ELISA antibodies were purchased from Agilent Technologies (Santa Clara, Calif., USA) and Szabo Scandic HandelsgmbH & Co KG (Vienna, Austria), for coating and detection respectively. All antibodies for CHO HCP ELISA were bought from Cygnus Technologies (Southport, N.C., USA). The detection substrate for ELISA, TMB Peroxidase EIA Substrate Kit, was bought from BIORad Laboratories Inc. (Hercules, Calif., USA). Samples of CHT I and II resin were provided by BIORad Laboratories Inc. rFVIII bulk drug substance (BDS) and rFVIII reference material, as well as rVWF bulk drug substance and rVWF reference material were provided by Baxalta Innovations GmbH.

### Acrylic-Glass-Based Prototype Equipment

Acrylic glass plates with different thicknesses were obtained from Evonik Industries AG (Essen Germany). Parts for assembly were cut from the plates with a Speedy 400 laser cutter (Trotec Laser GmbH, Marchtrenk, Austria) and glued with a mixture of 70% dichloromethane, 20% acetic acid glacial, 10% 4-hydroxy-4-methyl-2-pentanone and, or Acryfix192.

### Labware

Microtiter plates were purchased from Thermo Fisher Scientific (Waltham, Mass., USA). 15 and 50 mL reaction tubes were from Greiner AG (Kremsmünster Austria), 2 mL safe lock tubes from Eppendorf AG (Hamburg, Germany) and 1.5 mL reaction tubes from Sarstedt (Biedermannsdorf, Austria).

### Prototype Setup for Continuous Precipitation

The prototype setup consisted of the pump (Px) generating feed flow to the first vessel termed “surge tank”, which was equipped with an overhead stirrer (upward pitched blade impeller, 38 mm diameter) operated at ˜150 rpm. A second pump (P5) ensured flow from the surge tank through a tubular reactor to a 50 mL glass vessel, termed “CSTR”, equipped with an overhead stirrer (upward pitched blade impeller, 25 mm diameter) operated at ˜150 rpm. From the CSTR the process fluid was transported to a collection vessel or the prototype inclined plate settler (pump P8). Addition points for stock solutions of calcium and phosphate were placed between P5 and the TR. The addition of the stocks was operated by a syringe pump (P6 and P7). Both glass vessels were put on scales. A full list of the used equipment is given below (Table 9). Tubing was 1.5 mm ID silicone tubing (Reichelt Chemietechnik GmbH+Co.) except for the tubular reactor. Connections between individual tubing were made with Luer fittings (Cole Parmer). The precipitation was controlled using a custom programmed LabVIEW (v2018) tool.

TABLE 9
List of pieces of equipment installed as part of the prototype for continuous precipitation.

| | Equipment Type | Equipment ID | Supplier/Manufacturer |
|---|---|---|---|
| Prototype label | | | |
| Px | Pump | SP570 EC-BL-LD | Schwarzer Precision GmbH |
| Surge tank | Vessel | 100 ml EasyMax reactor | Mettler Toledo |
| Surge tank pH | Sensor | InLab ® Max Pro-ISM | Mettler Toledo |
| Surge tank balance | Sensor | Entris 6202i-1S | Sartorius AG |
| P4 | Pump | RegloDigital MS-4/12 | Cole-Parmer |
| P5 | Pump | SP570 EC-BL-LD | Schwarzer Precision GmbH |
| P6, P7 | Pump | KD Scientific Gemini 88 | Thermo Fisher |
| Tubular reactor | Vessel | ID 3 mm + static mixers | Custom |
| CSTR | Vessel | 50 ml EasyMax reactor | Mettler Toledo |
| CSTR pH | Sensor | InLab ® Micro Pro-ISM | Mettler Toledo |
| CSTR balance | Sensor | Entris 6202i-1S | Sartorius AG |
| P8 | Pump | RegloDigital MS-4/12 | Cole-Parmer |
| Periphery parts | | | |
| | pH meter | Seven Excellence S400 | Mettler Toledo |
| | Card holder | cDAQ-9174 | National Instruments |
| | Analogue output card | NI 9264 | National Instruments |
| | Analogue input card | NI 9207 | National Instruments |
| | Digital output | NI 9276 | National Instruments |

### Inclined Plate Settler Prototype

The lab-scale prototype inclined plate settler consisted of a stainless steel settling section with a 3D-printed bottom section and a custom acrylic glass top flow-collector. It was comprised of a single plate and equipped with a NED 605 vibration motor (Netter GmbH, Mainz-Kastel, Germany). Table 10 lists the measurements of the plate settler. The wash pump was a SP270 EC-BL-L 12V membrane pump (Schwarzer Precision). The sludge pump was a Masterflex L/S equipped with an Easy-Load II pump head (Cole Parmer, Vernon Hills, Ill., USA). The settler prototype was equipped with custom turbidity sensors with electromagnetic wiping function. The prototype was controlled via a custom software tool programmed in National Instruments LabVIEW (v2018). The National Instruments periphery equipment was identical to the one listed in Table 9.

TABLE 10
Inclined plate settler prototype measurements and data.

| | |
|---|---|
| Total volume [mL] | 630 |
| Total receiver section volume [mL] | 44.5 |
| Receiver section working volume [mL] | 22.3 |
| Plate width (l × b × h) [mm × mm × mm] | 50 × 24 × 500 |
| Inclination angle [deg] | 60 |

The bottom section had 8 wash fluid outlets that were equally spaced across the entire width of the plate (i.e. 5 cm). The length of the plate was 50 cm, the width was 5 cm for separating precipitate, but (contrary to Table 10) 5.5 cm for separating cells. There were no protruding structures spanning the depth of the bottom section. The suspension was supplied via 8 feed outlets that were also equally spaced at the very top of the bottom section.

### Methods

### Polyethylenglycol (PEG) Precipitation

PEGs of different molecular weights (2.000 to 20.000 Da) were used for precipitation of FVIII:VWF from CCSN. All experiments were carried out at 4° C. using in 15 mL Greiner tubes. PEG stock solutions were made in 50 mM HEPES, pH 7.5 with final concentrations of 50% (w/v) for PEG 2,000, 4,000, 6,000 and 8,000 and 40% (w/v) for PEG 10,000 and 20,000. Samples were mixed by end-over-end rotation at 3 rpm during incubation overnight. Precipitates were pelleted by centrifugation at 4000 rcf, 4° C. for 1 h using a Heraeus Multifuge X3 FR swing-out-rotor centrifuge (Thermo Fisher, Waltham, Mass., USA).

### PEG Precipitation—PEG Size Screening

A total volume of 5 mL of additives (PEG stock solution and buffer) were mixed with an equal volume of CCSN to final concentrations of 5, 12 and 19% PEG. The precipitates were dissolved in 5 mL 50 mM HEPES with 100 mM NaCl, pH 7.5 under constant mixing by end-over-end rotation over 24 h. Precipitation efficiency was estimated by SDS-PAGE.

### PEG Precipitation—PEG Precipitation Curves

PEG6,000, 8,000, 10,000 and PEG20,000 were used. The total volume was 10 mL, which consisted of 6.5 mL CCSN and 3.5 mL additives. Final PEG concentrations of 4 to 14% (w/v) were tested. After separation, the precipitates were dissolved in 3.25 mL 15 mM TRIS, 600 mM NaCl, pH 7.5. FVIII concentration in the dissolved precipitate was quantified using the high-throughput BLI-based method.

### Titration of CCSN

Cell culture supernatant (CCSN) was titrated using 0.1 M HCl or 0.1 M NaOH. The titration was performed in 250 mL Nalgene bottles with magnetic stirring (˜100 rpm) at 2-8 C. Samples were taken at pH 6 to 8.5 or 6 to 9.25, in increments of 0.25 or 0.5 units and incubated for 1 h at this pH value. Neutralization was performed by dilution of the samples in assay buffer for FVIII and VWF:Ag ELISA quantification, respectively. The CCSN was titrated unmodified and added with 16 g/100 g of a solution containing 20.6% (w/w) NaCl and 4.14% (w/w) CaCl₂.

### Calcium Phosphate Batch Precipitation

Batch precipitation was performed using thawed CCSN without and with pH modification by addition of 2 M TRIS, pH 7.75 at room temperature (RT) to a final concentration of 50 mM or 0.25 M NaOH. Calcium and phosphate were added as of stock solutions of 4 or 5 M and 0.2 or 0.4 M, respectively. Mixing was performed by end-over-end rotation, unless indicated otherwise. The precipitate was settled over night. The clarified precipitation supernatant was removed and the settled precipitate was dissolved by gradual addition of 1 M Citrate buffer pH 6.5 or pH 7.0 until the precipitate had fully dissolved. Completeness of dissolution was determined by visual inspection.

### Calcium Phosphate Batch Precipitation—Wash of Calcium Phosphate Precipitate

The wash buffers used are listed in Table 11 with the pH values set at RT (approx. 22° C.). Buffers were used at 2 to 8° C. Wash buffers B7 to B10 were used for washing precipitate in 50 mL glass separation funnels. The precipitate was added from the top, after which wash buffer was supplied from the bottom using a low flow produced by a peristaltic pump. The precipitate was allowed to settle into the wash buffer for ˜1.5 h. The precipitate suspension was collected through the bottom outlet. The dissolved precipitate samples were stored at <−60° C. until analysis. Each wash condition including the reference sample without wash buffer addition was tested in triplicate.

TABLE 11
List of buffers used for washing calcium phosphate precipitate after
precipitation from CCSN. B7 to B10 buffers for washing in separation
funnels with focus on yield and equal wash buffer density.

| | | | pH | Calcium | NaCl |
|---|---|---|---|---|---|
| Buffer No. | Buffering | Conc. | (at RT) | conc. | conc. |
| [—] | salt | [mM] | [—] | [mM] | [mM] |
| B7 | TRIS | 2 | 7.75 | 0 | 272 |
| B8 | TRIS | 2 | 7.75 | 4 | 258 |
| B9 | TRIS | 2 | 7.75 | 8 | 245 |
| B10 | TRIS | 2 | 7.75 | 12 | 231 |

### Calcium Phosphate Batch Precipitation—Precipitation Kinetic Studies

Precipitation kinetics were performed using an EasyMax102 synthesis workstation equipped with a 100 mL EasyMax glass reactor. Mixing was performed using a 25 mm diameter upward pitched blade impeller operated at 100 rpm. pH modification was achieved with 2 M TRIS (final pH 8.5) or 1 M NaOH (final pH 8.95). Experiments were performed at 4° C. with temperature control via the Easymax102 workstation. The target concentration for calcium was 15 mM and 2 mM for phosphate. Samples were taken between 1 and 60 minutes after phosphate stock addition. All samples were immediately centrifuged for 1 min, 4000 rcf, 4° C. using a 5415R benchtop centrifuge (Eppendorf AG, Hamburg, Germany). The precipitation supernatant was analyzed for presence of VWF and FVIII.

### Calcium Phosphate Batch Precipitation—Precipitation Kinetic Studies—Mixing Studies

Mixing studies were performed in the reactor described below with differently sized upward pitched blade impellers. Impeller diameters were 25 and 38 mm, respectively. Stirrer speed was tested in the range from 50 to 500 rpm. The reactor was filled with 70 mL H₂O. A pulse of 0.35 mL 5 M CaCl₂ was dosed and mixing was monitored using a conductivity sensor.

### Calcium Phosphate Batch Precipitation—Binding Studies of FVIII:VWF to Calcium Phosphate Surfaces

CCSN (25 mL) was transferred to 50 mL glass beakers and added with ex-situ formed precipitate and CHT resins (Table 12). A reference sample was in situ precipitated. pH was modified by addition TRIS (final concentration 50 mM). CCSN incubated with CHT resin was added with 4.5 mM of phosphate. Samples were incubated with calcium phosphate for approx. 7 h (constant mixing by magnetic stirrer ˜150 rpm, at 2-8° C.).

TABLE 12
Calcium phosphate precipitates and resins incubated with CCSN
including used matrices for precipitation and buffers used.

| Calcium phosphate surface | Type [—] | Matrix | pH modifier | Elution buffer |
|---|---|---|---|---|
| In situ formed | Precipitate | CCSN | TRIS | 1.2M citrate, pH 7.5 |
| In situ formed | Precipitate | CCSN | TRIS | 3.5M NaCl, 0.3M CaCl₂ |
| Ex situ formed | Precipitate | H₂O | TRIS | 1.2M citrate, pH 7.5 |
| | | Amount used | Equilibration buffer | |
| Bio-Rad CHT I | HAP resin | 0.5 g | 10 mM TRIS, 5 mM | 10 mM TRIS, 0.5M |
| Bio-Rad CHT II | HAP resin | 0.5 g | Na₂HPO₄, pH 8.5 | Na₂HPO₄, pH 8.5 |

### Reactor Configurations for Continuous Precipitation of Calcium Phosphate

Calcium phosphate was continuously precipitated from 50 mM TRIS buffer or CCSN by addition of 2 mM Na₂HPO₄ and 15 mM calcium chloride in reactor configurations listed in Table 13 at a mean residence time of 9 minutes. The CSTR was an EasyMax102 equipped with a 100 mL glass reactor vessel equipped with a 38 mm diameter pitched blade upward impeller, operated at 100 rpm. The tubular reactors consisted of 4.8 mm inner diameter tubing with static mixers. The feed and harvest flow, i.e. flow to and from the reactor, were operated with peristaltic pumps (Ismatec RegloDigital, 1.85 mm ID tubing, ColeParmer, Vernon Hills, Ill., USA).

TABLE 13
Reactor configurations used for continuous precipitation
of calcium phosphate with reactor volume and
corresponding volumetric flow rates.

| Reactor | Reactor Volume [mL] | Volumetric flow rate [mL/min] |
|---|---|---|
| CSTR | 90 | 10 |
| TR | 91.9 | 10.2 |
| TR + CSTR | 105.3 | 11.7 |

### Reactor Configurations for Continuous Precipitation of Calcium Phosphate—Tracer Step Experiments in Continuous Reactors

Step experiments were performed with H₂O and 1 M NaCl or calcium phosphate precipitate formed in 50 mM TRIS and clarified precipitation supernatant. The reactors tested were described in the above section. The NaCl concentration was monitored by conductivity measurement in the reactor or in a custom-built flow cell at the reactor outlet. Concentration of calcium phosphate flocks was measured with custom-built turbidity sensors. The tracer concentrations were normalized for the concentration reached at the end of the experiment. By derivatization of the normalized tracer data, the residence time distribution was obtained.

### Reactor Configurations for Continuous Precipitation of Calcium Phosphate—Determination of Settling Velocity

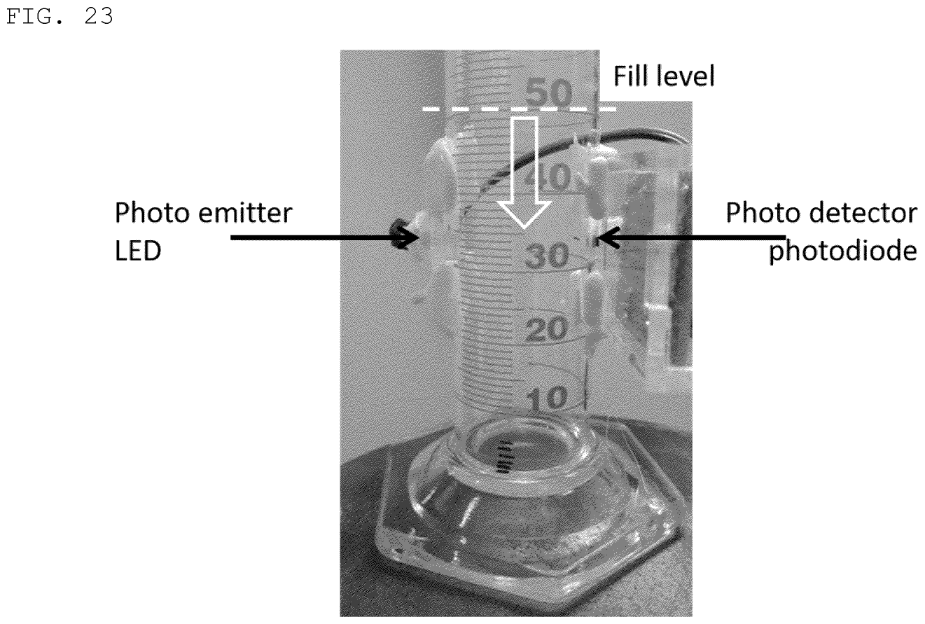

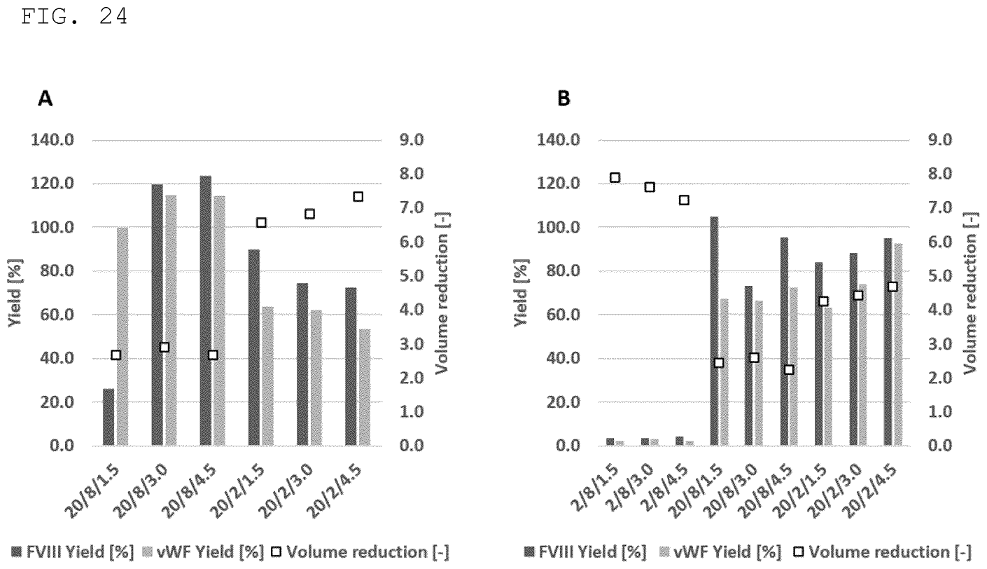

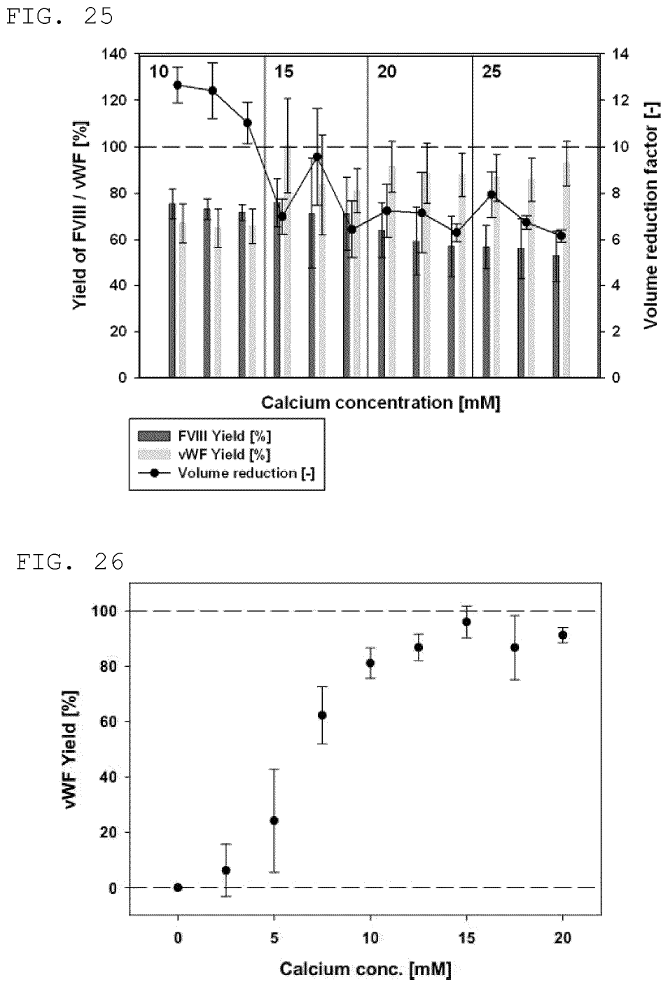

The settling velocity was determined for batch experiments with 3, 9 and 45 min mixing times and for continuous precipitation experiments (mean residence time 9 min). Continuous precipitation in the CSTR was started by batch precipitation and a continuous precipitation approach. For the batch start, the reactor was filled with phosphate-supplemented buffer and calcium was added before the continuous precipitation was started. For the experiment with continuous start, the reactor was initially empty and both the buffer and the calcium stock were dosed in continuous mode. The settling was monitored using a custom-built device made from a 100 mL glass measuring cylinder equipped with an optical sensor based on a photodiode emitter and detector (see FIG. 23). The sample (50 mL) was transferred to the measuring cylinder and turbidity was monitored for 30 minutes. Each experiment was at least performed in triplicate.

### Continuous Precipitation of Calcium Phosphate

Preliminary, continuous precipitation experiments were performed using a protein free model system (50 mM TRIS buffer, pH 8.5) and thawed CCSN. Calcium and phosphate concentrations were 15 and 2 mM, respectively. Addition of phosphate and pH modification to pH 9.0 were performed in batch, while calcium addition was performed in continuous mode. For experiments with CCSN, the CSTR was empty at the beginning, while tubing and TR were filled with buffer (50 mM TRIS, pH 8.5, 2 mM phosphate). Samples were collected at the reactor outlet in 0.5 reactor volumes intervals with shorter intervals at the end of the experiment. The precipitate suspension was allowed to settle and the supernatant was removed. The precipitate was dissolved by gradual addition of 1.2 M citrate, pH 7.5.

### Automated, Continuous Precipitation of Calcium Phosphate

Automated, continuous precipitation was performed using the prototype setup described in the above materials section “Prototype setup for continuous precipitation”. The hardware was complemented by a custom-built software tool programmed in National Instruments LabView. The starting material for the continuous precipitation was either 10 mM TRIS, pH 7.4 with 100 mM NaCl or CCSN. Experiments with buffer were performed at room temperature; experiments with CCSN were performed at 2 to 8° C. Calcium target concentration was 14.88 mM (stock concentration 4000 mM). Phosphate target concentration was 2.68 mM for protein free buffer and 1.98 mM for CCSN (stock concentration 200 mM). The precipitate was dissolved by addition of 0.5 M citrate stock pH 7.0. Before every experiment with CCSN, the pH sensors were calibrated and the pumps and all tubing were flushed with the corresponding process solutions. The surge tank and the CSTR were pre-filled with CCSN. Three continuous experiments lasting for approx. 4.5 to 5 h were performed with the equipment as described above. One additional experiment in the same length was performed without the tubular reactor. One precipitation experiment lasting for 24 h and another 24 h run with the precipitation integrated with the prototype inclined plate settler were performed. During the continuous runs, samples were taken from the surge tank, from the CSTR outlet stream, from the overflow of the inclined settler and from the discharged material. Precipitate suspensions were settled at 2-8° C. after which the precipitation supernatant or the wash buffer was sampled and the precipitate was dissolved. Samples were stored at <−60° C. until analysis.

### Automated, Continuous Precipitation of Calcium Phosphate—pH Control

pH modification was performed in the surge tank by addition of 0.25 M NaOH. The surge tank pH was measured and depending on the input pH a proportional or constant output was generated via the peristaltic pump (P4) controlled by the software. The output flow rate level in dependence on the input pH value is given in Table 14.

TABLE 14
pH control in the automated, continuous precipitation with parameters,
default values and control mechanisms implemented.

| Control parameter | Default value | Control mechanism programmed in the software |
|---|---|---|
| Nominal flow NaOH | 0.04 | =flow rate of NaOH if: Lower limit pH < ST pH < Upper limit |
| [mL/min] | | pH |
| p-value | 0.3 | If: current pH < Lower limit pH → (Target pH − ST pH)*p- |
| | | value = flow rate of NaOH |
| Critical pH | 8.3 | If: ST pH < critical pH → Nominal Main = 0 |
| Lower limit pH | 8.4 | If: ST pH < Lower limit pH → use proportional controller. |
| | | If: ST pH > Lower limit pH → use Nominal flow NaOH |
| | | [mL/min] |
| Target pH | 8.48 | — |
| Upper limit pH | 8.49 | If: ST pH < Upper limit pH → use Nominal flow NaOH |
| | | [mL/min] |
| | | If: ST pH > Upper limit pH → Stop P4 |

ST = surge tank.
p-value = proportional value for control.

### Automated, Continuous Precipitation of Calcium Phosphate—Volume Control—Surge Tank

The current liquid volume in the surge tank (ST fill) was monitored based on gravimetric measurements. Dependent on the ST fill relative to the control levels, the outflow from the surge tank was controlled. The sum of P5, P6 and P7, was regulated together (Nominal Main).

TABLE 15
Parameters and Limits for volume control in the surge tank
of the automated, continuous precipitation prototype.

| | Default | | |
|---|---|---|---|
| Control parameter | value | Unit | Control mechanism programmed in the software |
| ST upper alarm 2 | 130 | mL | If: ST fill > ST upper alarm 2 → Nominal main + Nominal |
| | | | increase + Nominal increase 2 |
| ST upper alarm 1 | 120 | mL | If: ST upper alarm 2 > ST fill > ST upper alarm → Nominal |
| | | | Main + Nominal increase |
| ST target level | 110 | mL | If: ST lower alarm 1 < ST fill < ST upper alarm 1 → Nominal |
| | | | Main |
| ST lower alarm 1 | 100 | mL | If: ST Sower alarm 2 < ST fill < ST lower alarm → Nominal |
| | | | Main − Nominal decrease |
| ST lower alarm 2 | 90 | mL | If: ST fill < ST lower alarm 2 → Nominal Main = 0 |
| Nominal Main | 3.5 | mL/min | =P5 + P6 + P7 |
| Nominal decrease | 0.5 | mL/min | — |
| Nominal increase | 0.5 | mL/min | — |
| Nominal increase 2 | 0.5 | mL/min | — |

### Automated, Continuous Precipitation of Calcium Phosphate—Volume Control—CSTR

Fill level control in the CSTR was done analogous to the above section “Volume control—surge tank”. However, the up and down regulation of the outflow was realized by modification of the pump speed.

TABLE 16
Parameters and Limits for volume control in the CSTR
of the automated, continuous precipitation prototype.

| | Default | | |
|---|---|---|---|
| Control parameter | value | Unit | Control mechanism programmed in the software |
| CSTR upper alarm | 38.5 | mL | If: CSTR fill > CSTR upper alarm 2 → Nominal flow + Outlet |
| 2 | | | increase 2 |
| CSTR upper alarm | 36.75 | mL | If: CSTR upper alarm 2 > CSTR fill > CSTR upper alarm → |
| 1 | | | Nominal Flow + Outlet increase |
| CSTR target level | 35 | mL | If: CSTR lower alarm 1 < CSTR fill < CSTR upper alarm 1 → |
| | | | Nominal Flow |
| CSTR lower alarm 1 | 33.25 | mL | If: CSTR lower alarm 2 < CSTR fill < CSTR lower alarm → |
| | | | Nominal Flow − Outlet decrease |
| CSTR lower alarm 2 | 31.5 | mL | If: CSTR fill < CSTR lower alarm 2 → Nominal Flow = 0 |
| Nominal flow | 36 | rpm | — |
| Outlet decrease | 30 | % | — |
| Outlet increase | 20 | % | — |
| Outlet increase 2 | 20 | % | — |

### Continuous Solid-Liquid Separation

Continuous solid-liquid separation was performed using the prototype inclined plate settler described in the above materials section “Inclined plate settler prototype”. The system was controlled using a custom software programmed in National Instruments LabVIEW. All experiments were run with the “run program” mode of the software. The vibration motor was set to an interval of 2 min with 3 s of vibration. The turbidity sensor wiping was set to 0.25 min interval, 7 Hz and 2 s duration.

### Continuous Solid-Liquid Separation—Settling of Precipitate in Separation Funnels

Precipitate suspension was settled in a 1 L glass separation funnel. The suspension, which was added from the top, consisted either of 50 mM TRIS, 165 mM NaCl, pH 8.6 precipitated with 15 mM CaCl₂ and 2 mM phosphate, or of CCSN adjusted to pH 8.5 with 0.1 M NaOH precipitated with 15 mM CaCl₂ and 2 mM phosphate. Wash buffer in varying composition was supplied from the bottom of the separation funnel using an Ismatec RegloDigital peristaltic pump (Cole Parmer, Vernon Hills, Ill., USA) at low flow rate. The precipitate was settled for approx. 45 minutes. Mixing between the phases was judged by visual inspection facilitated by the addition of 100 mg/L Patent Blue V to the wash buffer. All buffers were 2 mM TRIS, pH 8.25 with NaCl and CaCl₂ concentrations as listed in Table 17.

TABLE 17
Precipitate suspension and wash buffer combinations
with NaCl and CaCl₂ tested.

| | Tested with | NaCl conc. | CaCl₂ conc. |
|---|---|---|---|
| Buffer No. | precipitate from2 | [mM] | [mM] |
| W1 | Buffer | 350 | 0 |
| W2 | Buffer | 300 | 0 |
| W3 | Buffer | 250 | 0 |
| W4 | Buffer | 270 | 0 |
| W5 | CCSN | 247 | 0 |
| W6 | CCSN | 257 | 0 |
| W7 | CCSN | 265 | 0 |
| W8 | CCSN | 272 | 0 |
| W9 | CCSN | 221 | 12 |
| W10 | CCSN | 231 | 12 |

### Continuous Solid-Liquid Separation—Screening of Operation Conditions for Inclined Plate Settler Operation

Batch precipitate was produced in 10 mM TRIS buffer, pH 8.5 with 100 mM NaCl, precipitated with 15 mM CaCl₂ and 2.7 mM Na₂HPO₄. The precipitate was prepared once directly before the experiment or fresh every two hours and was kept in suspension by agitation using a magnetic stirrer. Feed flow rate was 3.5 mL/min during all runs. Wash buffer composition was 2 mM TRIS, 252 mM NaCl, 6 mM CaCl₂, pH 8.2. Patent Blue V and Tryptophan were quantified by absorbance measurements at 620 and 280 nm, respectively. Absorbance measurements were performed on a Tecan InfiniteM200 Pro plate reader in 96-well format (Tecan Trading AG, Männedorf, Switzerland).

### Continuous Solid-Liquid Separation—Screening of Operation Conditions for Inclined Plate Settler Operation—Screening of Discharge Flow Rate

Wash buffer and sludge pump flow rates were set to either 20, 40 or 60 mL/min. The discharge volume was held constant by varying the time of the discharge interval. Wash buffer was supplemented with 25 mg/L Patent Blue V as a tracer with no tracer in the feed or the feed was supplemented with 100 mg/L of Tryptophan with no tracer in the wash buffer. The discharge interval was set to 15 min. The discharge volume was 40 mL when the tracer was in the wash buffer and 44 mL when the tracer was in the feed. The additional 4 mL were obtained because the sludge pump was operated slightly longer to lower the wash buffer front.

### Continuous Solid-Liquid Separation—Screening of Operation Conditions for Inclined Plate Settler Operation—Screening of Discharge Interval

The discharge flow rate was held constant at 40 mL/min and the discharge volume was 45 mL for all intervals tested. Out of these 45 mL, 40 mL were discharged with simultaneous flow of the wash and sludge pump and an additional 5 mL were discharged with sludge flow only. The discharge interval was varied from 30 to 60 min. The wash buffer was supplemented with 25 mg/L Patent Blue V.

### Continuous Solid-Liquid Separation—Screening of Operation Conditions for Inclined Plate Settler Operation—Reduction of Discharge Volume

The discharge flow rate was held constant at 40 mL/min and the discharge interval set to 30 min. The discharge volume was 22.8 and 12.8 mL. Out of the total discharge volume, 4 mL were discharged with sludge flow only, while the rest was discharged at simultaneous flow of both wash and sludge pump.

### Continuous Solid-Liquid Separation—Separation of Continuously Precipitated Calcium Phosphate Using the Inclined Plate Settler

The feed buffer was a 10 mM TRIS, pH 7.4 buffer with 100 mM NaCl. pH modification was fully automated within the continuous precipitation by addition of 0.25 M NaOH. Precipitation was initiated by addition of calcium and phosphate stock solutions (4 M and 0.4 M, respectively). The outlet of the continuous precipitation setup was connected to the inlet of the single plate inclined settler. The settler was pre-filled with precipitation supernatant generated by clarification of 10 mM TRIS, 100 mM NaCl, pH 8.5+15 mM CaCl₂+2.7 mM Na₂HPO₄. Supernatant clarification was achieved by settling of the precipitate and a subsequent two-stage filtration (1.2 μm and 0.45 μm). The settler was operated with a discharge flow rate of 40 mL/min, an interval of 30 min and a discharge volume of 12.8 mL. The feed flow rate was automatically controlled within the continuous precipitation.

Titration of Calcium with Citrate

Solutions consisting of 10 mM CaCl₂ and 20 mM buffer component depending on the target pH value were titrated with 1 M citrate stock solutions set to the same pH as the bulk solution. Solutions at pH 5.0 and 5.5 were buffered with acetate, pH 6.0 and 6.5 were buffered with BIS-TRIS, pH 7.0, 7.25 and 7.5 were buffered with HEPES, pH 8.0 was buffered with TRIS. 100 mL calcium containing solutions were added with 2 mL of Mettler Toledo™ ISA solution prior to any measurement. Standard curves were made using the same buffer components with calcium concentrations from 0.1 to 100 mM. Titrations were performed at an addition rate of 0.1 mL/min. Measurements were performed at room temperature and at 4° C. using an EasyMax102 synthesis workstation for temperature control and mixing. Free calcium was monitored in real time using a calcium specific electrode (perfectION™ comb Ca Lemo combination electrode, Mettler Toledo, Columbus, Ohio, USA).

### HPLC Measurements of Citric Acid

Quantification of citric acid by HPLC measurements was performed. The HPLC method was previously described by Blumhoff et al. and Steiger et al. (References 9 and 10, respectively).

### Offline Calcium Quantification

Calcium concentration was measured using a calcium colorimetric kit in 96-well plate format. The standard and all samples were diluted by 1:2 serial dilutions with water resulting in concentrations from 2.5 to 0.08 mM calcium. The quantification was performed according to the procedure described by the supplier. Measurements were performed using a Tecan InfiniteM200 Pro plate reader (Tecan trading AG, Männedorf, Switzerland).

### Phosphate Quantification

Quantification of phosphate was performed using the method described by Satzer et al. (Reference 11).

### FVIII Activity Quantification

FVIII activity was determined using the Chromogenix Coatest SP4 VIII kit in a 96-well plate format. The assay buffer and reagent mix (CaCl₂, phospholipid and FIX+FX mixture) were prepared fresh prior to every analysis. Standard (reference material; Baxalta Innovations GmbH, Orth/Donau, Austria), internal control and samples were diluted in 1:2 steps and transferred to the measurement plate. The reagent mixture was added to the samples and incubated for 5 min at 37° C. For detection, the chromogenic substrate was added and the absorbance was read at 405 nm, at 37° C. over 5 minutes with measurements in intervals of 30 s using a Tecan InfiniteM200 Pro plate reader (Tecan trading AG, Männedorf, Switzerland).

### VWF Quantification

The VWF concentration in the samples was quantified using an antigen ELISA. MAXISORP™ plates were coated with rabbit anti-VWF antibody (DAKO A0082, diluted 1:600). Plate washing was performed using a Tecan Hydroflex plate washer (Tecan Trading AG) and 1×TBS buffer. Standards, internal controls and samples were diluted with 1×TBS-T+0.1% HSA sample buffer in 1:2 steps. The samples were detected with rabbit anti-VWF HRP conjugated antibody (DAKO P0226, diluted 1:40.000 with DPBS+10% rabbit serum). For detection TMB Peroxidase EIA Substrate Kit was used. Results were obtained by absorbance measurement at 490 nm using a Tecan InfiniteM200 Pro plate reader.

### HCP Quantification

CHO HCPs were quantified using the ELISA method described by Sauer et al. (Reference 12).

### DNA Quantification

Quantification of double stranded DNA was performed using Picogreen™ assay as previously described by Satzer et al. (Reference 11).

### SDS-PAGE

Reducing SDS-PAGE was performed using 3-8% TRIS-Acetate gels run with 1× TRIS-Acetate buffer. Samples were prepared by mixing of 1 part 2 M DTT, 1 part sample buffer (NuPAGE LDS Sample Buffer (4×)) and 3 parts sample, heated to 95° C. for 10 minutes. For determination of band size, HiMark™ pre-stained standard was used. Bands were detected using silver stain.

All results in the following examples 7-10 were obtained by processing or analysis of rFVIII (octocog alfa) and rVWF (vonicog alfa) as described in the materials section above.

### Example 7: Batch Precipitation of the FVIII:VWF Complex

In order to establish a continuous capture step of the recombinant FVIII:VWF complex based on precipitation, batch precipitation experiments were performed. To that end, the precipitation capability of different precipitants was tested.

### Example 7.1: Batch Precipitation Using Polyethylenglycol (PEG)

PEG was the first precipitant investigated regarding suitability to precipitate the FVIII:VWF complex from cell culture supernatant (CCSN). PEG was tested over a wide range of PEG molecular weights from PEG2,000 to PEG20,000. The precipitation supernatant (SN) and the dissolved precipitate samples (DP) were checked for the presence of the target proteins by SDS-PAGE. The SN samples were compared to CCSN diluted 1:2. For the lower molecular weight PEGs the protein concentration in the SN and the reference was very similar, indicating no precipitation. With increasing molecular weight and increasing PEG concentration, the amount of protein in the SN samples decreased and was increased in corresponding DP samples. The threshold with regard to PEG molecular weight appeared to be 6,000 kDa. In samples precipitated with PEG6,000 or higher at 12 and 19% (w/v) the target proteins were detected in the dissolved precipitates. However, at high PEG concentrations the solutions became viscous. Moreover, efficient sedimentation of the precipitates depended on centrifugation.

### Example 7.2: Batch Precipitation Using Calcium Phosphate

### Batch Precipitation Using Calcium Phosphate

The feasibility of calcium phosphate precipitation for capture of the FVIII:VWF complex was first tested from 2 to 20 mM for calcium and 2 to 8 mM for phosphate. All combinations were tested with and without pH modification prior to precipitation. Herein pH modification was performed by addition of a 2 M Tris stock solution with a pH of 7.75 at ambient temperature (˜21° C.) in order to yield a final concentration of 50 mM Tris in the cell culture supernatant and a target pH of approx. 8.3 at 4° C. Calcium phosphate solubility is dependent on the solution pH. In the unmodified CCSN, only two out of four combinations resulted in the formation of solid calcium phosphate, while three out of four combinations did when the CCSN pH was modified. The analytical results and the calculated volume reduction factor for the unmodified CCSN are shown in FIG. 24A, the results for the pH modified CCSN in FIG. 24B. For all conditions, an inverse relationship between yield and volume reduction was observed. The protein yield correlated directly to the precipitate amount. Increasing concentrations of calcium and phosphate resulted in increased precipitate formation, i.e. increased yield. Calcium phosphate flocks typically presented as a loose network that could easily be disturbed by agitation and showed limited compression during settling. Therefore, the volume reduction was governed by the amount of precipitate present. The different citrate amounts had very little to no influence on product yield and volume reduction. The selection of conditions was made with focus on product yield. The samples obtained after pH modification with 20 mM calcium and 2 mM phosphate exhibited high yield and intermediate volume reduction. This condition was chosen as a starting point for further investigations.

In total four different calcium concentrations (10, 15, 20 and 25 mM) and three phosphate concentrations (1.5, 2.0 and 2.5 mM) were tested with 5 replicates per condition. The pH of the thawed CCSN was modified and stabilized by addition of TRIS. The solution pH after modification was approx. 8.3. The higher number of replicates allowed a statistical evaluation of the data to check for significant differences between the precipitation conditions by analysis of variances (ANOVA). The data (results plotted in FIG. 25) indicated a statistically significant difference in VWF yield between 10 and 15 mM calcium. Phosphate concentration had no significant influence within the tested range. Notably, the concentration range for phosphate was very narrow, which made large differences less likely. For FVIII yield there were no significant differences detected at all. These results indicated a calcium dependent precipitation behavior of VWF within the tested concentration range. The influence of calcium concentration on VWF was investigated in more detail. At constant phosphate concentration and pH, calcium concentration was increased to 20 mM. Here, only VWF concentration in the dissolved precipitate was determined, because no influence of calcium concentration on FVIII yield was expected based on the previous results. The yield of VWF followed a sigmoid trend and reached a plateau at calcium concentrations ≤12.5 mM (see FIG. 26). The average yield obtained between 12.5 and 20 mM was 90.2%. Including a safety margin of 20%, 15 mM calcium was chosen as the target concentration for the precipitation of FVIII:VWF complex from CCSN using 2 mM phosphate, pH 8.3 (TRIS buffered).

The calcium and phosphate concentrations required for precipitation were determined using TRIS for pH modification, which provided buffer capacity. The precipitation of calcium phosphate was accompanied by a release in H⁺-ions. In the absence of a buffer system, a drop in pH was observed. Because of the pH dependence of the calcium phosphate precipitation, the protein yield could be impacted by a drop in pH. Therefore, precipitation was investigated over a range of starting pH values achieved by modification with NaOH. In the dissolved precipitate, VWF concentration increased with increasing starting pH. FVIII yield first increased with more alkaline pH and then decreased again at pH >8.5. The volume reduction decreased with increasing solution pH before precipitation (FIG. 27). In order to maximize yield of FVIII and VWF, pH 8.5 was defined as target pH before precipitation.

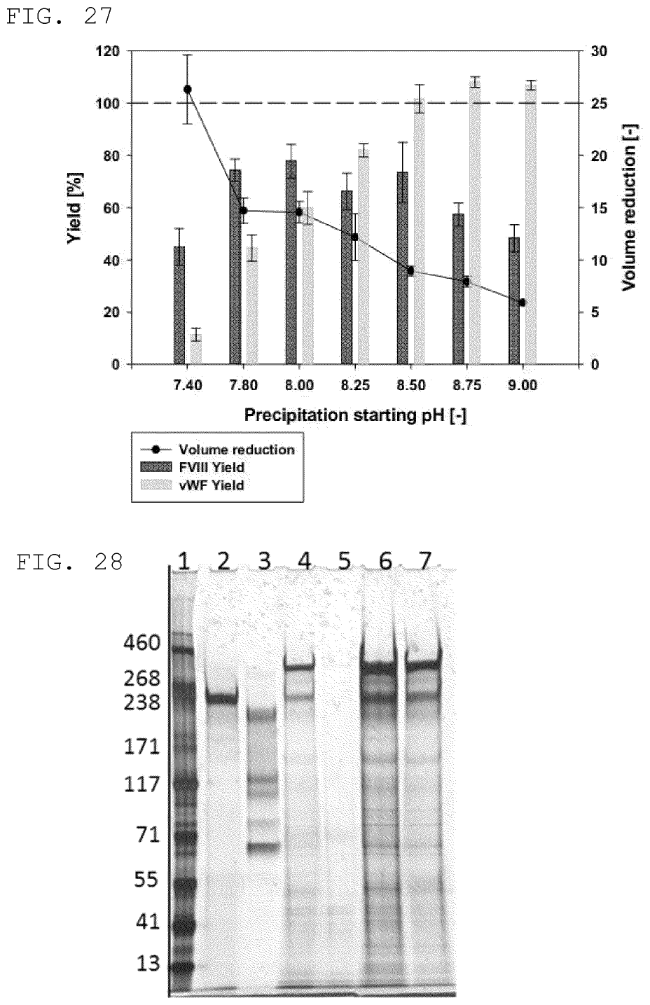

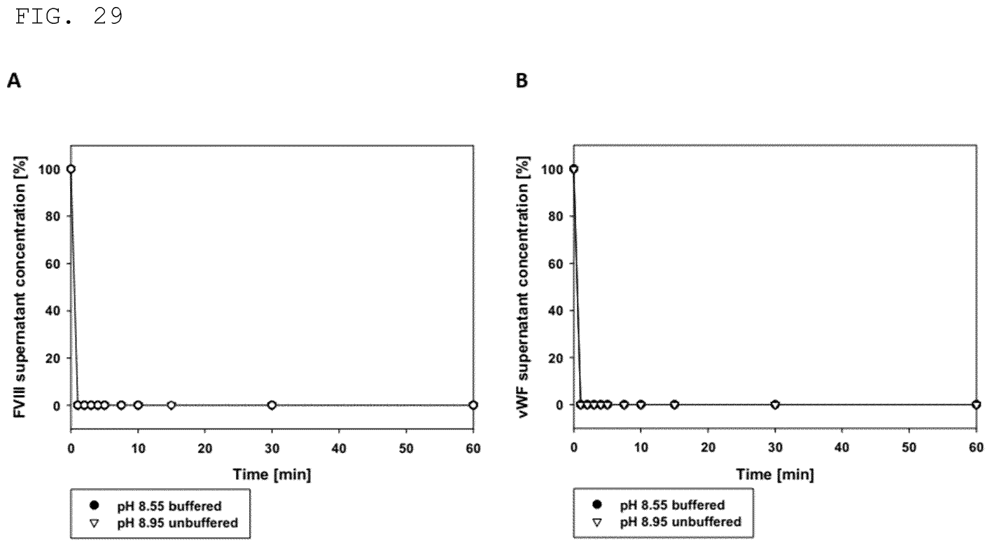

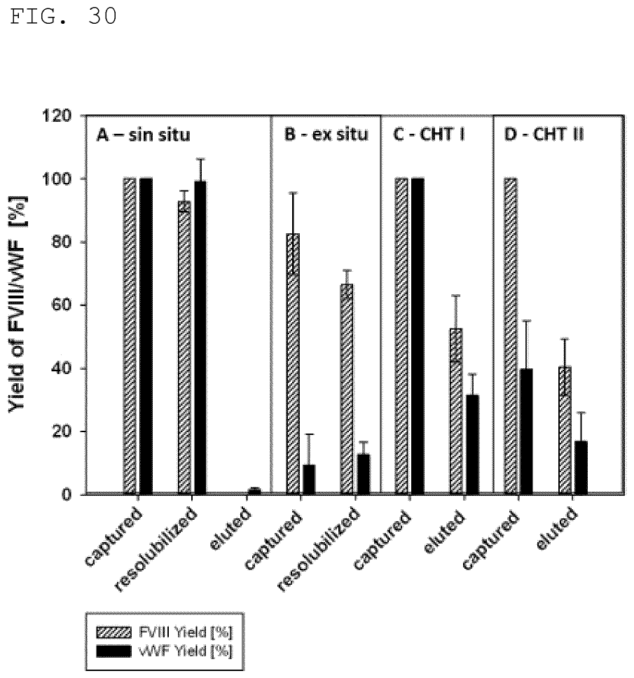

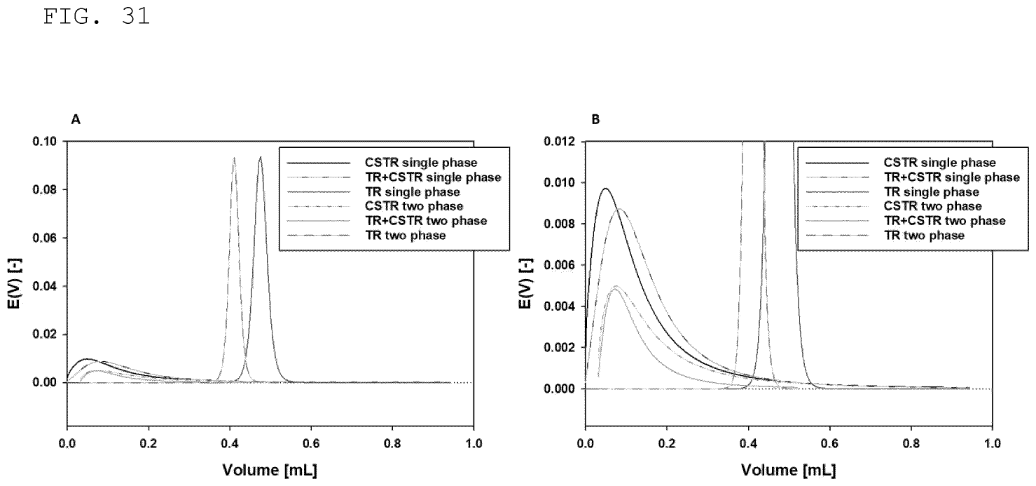

FIG. 28 shows a picture of an SDS-PAGE gel of CCSN precipitated under optimized conditions. The vast majority of proteins was depleted from the precipitation supernatant (lane 5), while the concentration in the dissolved precipitate (lane 6) was clearly increased relative to the starting material (lane 4).

### Batch Precipitation Kinetics for FVIII:VWF Calcium Phosphate Precipitation

For precipitation, kinetics are dependent on mass transfer, which is in turn dependent on the rate of mixing. In order to ensure homogenous precipitation conditions efficient mixing had to be ensured throughout the entire sample. Mixing in the CSTR intended for kinetic studies was investigated using two sizes of an otherwise identical upward pitched blade impeller operated at increasing stirrer speeds. The mixing time for each impeller size and stirrer speed was determined in “time until constant conductivity” after a pulse addition. With the smaller diameter impeller, significantly longer mixing times were observed (see Table 18). At 100 rpm, a homogeneous solution was obtained in less than one minute. Higher impeller speeds were excluded to prevent excessive, unnecessary shear stress.

TABLE 18
Mixing times in EasyMax102, 100 mL glass reactor with
upward pitched blade impellers (25 and 38 mm diameter).

Mixing time [s]

| Stirrer speed [rpm] | Ø 25 mm stirrer | Ø 38 mm stirrer |
|---|---|---|
| 50 | 192 | 44 |
| 100 | 54 | 12 |
| 175 | 25 | 5 |
| 250 | 21 | 4 |
| 500 | 6 | 2 |

For the precipitation kinetics the supernatant protein concentration was quantified relative to a control sample (i.e. CCSN), which represented the 100% reference. FVIII and VWF could not be detected in any of the supernatant samples taken after the precipitation, independent of the starting pH and the mode of pH modification. The concentration in the precipitation supernatant dropped to zero in both panel A and B of FIG. 29. These data indicated a rapid mode of removal of the FVIII:VWF complex from CCSN by calcium phosphate precipitation. Technically speaking, the precipitation kinetic, i.e. a time dependent behavior, was not observable under the conditions applied. Nevertheless, based on these results, removal from the CCSN was confirmed. However, the precipitate was not analyzed in this case, which left the mass balance incomplete. The possibility of degradation of the FVIII:VWF complex at elevated pH values was investigated and only minor degradation was observed (see below). These findings support the hypothesis, that FVIII and VWF would be found in the precipitate if removed from the precipitation supernatant.

### Mechanism of FVIII:VWF Capture by Calcium Phosphate

After precipitation, the product molecules were released from the calcium phosphate precipitate by dissolution of the precipitate. Consequently, the release or recovery step was independent of the capture mechanism. It has been speculated on a specific rather than a general precipitation mechanism. This assumption was based on the partial removal of HCP, while the antibody remained in solution. In principle a number of mechanisms such as co-precipitation, inclusion or adsorption could be the underlying phenomenon for the capture process proposed within these examples. In adsorption, the protein would be retained on the calcium phosphate surface and could be eluted without dissolution of the precipitate. If the protein was captured by co-precipitation, it would be integrated into the formed precipitate in a coordinated way. For inclusion, random integration of the protein entrapped within or in between the flocks would be assumed. Both co-precipitation and inclusion would require dissolution of the precipitate for release of the product.

To test for adsorption, wet calcium phosphate (ex situ formed calcium phosphate) as well as the chromatographic resins CHT I and II were added to the cell culture supernatant and incubated under constant mixing to prevent settling and ensure sufficient mass transfer. In the case of wet precipitate 18 and 90% of FVIII and VWF, respectively, remained in the CCSN and did not adsorb to the precipitate. The yield after dissolution was 66.1 and 11.5% for FVIII and VWF, respectively (FIG. 30, Panel C). With the chromatographic hydroxyapatite resin CHT I complete product capture was achieved, with yields in the elution fraction of 52% for FVIII and 31% for VWF (FIG. 30, Panel D). Here 48 and 69% of the initially present protein was not recovered, therefore it was assumed the products remained bound to the resin. With the second type of hydroxyapatite resin (CHT II) adsorption of FVIII was complete, while a significant amount of 60% of VWF was not captured. Yield in the elution fraction was 40% for FVIII and 17% for VWF (FIG. 30, Panel D). Recovery for FVIII and VWF was 40 and 67° %, respectively. These results showed FVIII could be adsorbed to fresh calcium phosphate and chemically similar solid phases. VWF was only adsorbed onto CHT I, but not onto the other resin nor on the ex situ formed calcium phosphate. Elution from in situ formed calcium phosphate precipitate was tested, but failed. The product concentration found in the undissolved in situ formed precipitate was in the same range as if the precipitate would have been dissolved (FIG. 30, Panel B). In these experiments, FVIII was efficiently adsorbed while VWF partially remained in solution. In this case, one must assume, FVIII and VWF were no longer in complex with each other. Activation of VWF by shear stress, e.g. as a result from mixing, causing a lower affinity for FVIII would explain disruption of the complex. To test the influence of the magnetic stirrer, calcium phosphate was precipitated in situ and mixed for several hours. In the stirred sample yield was 75% and 90% for FVIII and VWF, respectively. In the inversion mixed reference sample 93% of FVIII and 99% of VWF were recovered. Here, yield equally decreased for both FVIII and VWF. To account for shear introduced by the agitation with the magnetic stirrer, a stirred control with unmodified cell culture supernatant was included. Relative to the stagnant control sample, FVIII activity decreased by 20%, while VWF concentration was 10% lower. Experiments that included magnetic stirring were related to these reference values, while all others were related to the stagnant control sample. Independently of why FVIII and VWF did not behave as if they were a single entity for ex situ formed calcium phosphate and CHT II, the results indicated an adsorption based mechanism for FVIII. It was captured regardless of the solid phase, with varying degrees of efficiency though. VWF could be adsorbed to CHT I and partly adsorbed to CHT II, but not to ex situ formed calcium phosphate precipitate. Therefore, adsorption could only be part of the mechanism of VWF capture. Here, a general inclusion mechanism seemed more likely, as capture upon precipitate formation in the cell culture supernatant was highly efficient. The fact that the product complex could not be eluted from the precipitate also pointed at entrapment of the proteins.

For the in situ precipitated samples that were subsequently dissolved, the yield of HCPs and DNA in the dissolved precipitate were determined. HCP yield in the dissolved precipitates was 30%, which means 70% of the originally present HCPs were removed in the calcium phosphate precipitation step. Double stranded DNA was largely precipitated with the product. Yield for DNA was 79%, which was not surprising given the fact that calcium phosphate precipitation has previously been used for removal of DNA in an antibody process.

### Example 8: Continuous Precipitation of FVIII:VWF

For precipitation, transfer of the unit operation from batch to continuous mode was performed. This requires continuous mixing of the starting material with the precipitation reagent and provision of sufficient residence time for the precipitation to be complete. Thus, for the transfer to continuous operation a suitable reactor for continuous precipitation of calcium phosphate was required.

### Selection of a Reactor Configuration for Continuous Precipitation of FVIII:VWF

For continuous precipitation of the FVIII:VWF complex from CCSN, a reactor was chosen from three different reactor configurations: a CSTR, a tubular reactor (TR) and a combination of TR and CSTR (TR+CSTR). The reactors in question were characterized by (1) their RTD based on tracer step experiments, (2) by the settling velocity of the precipitate, (3) product yield produced by a specific reactor configuration and (4) considerations regarding practical realization. The selection was made using a decision matrix, in which for each category a total of 3 points were awarded. The reactor performing best in a category was given 2 points and the second best was given 1 point. If two reactors were performing equally well, both reactors were given 1.5 points. The reactor with the highest sum of points was selected. The RTD of the different reactor configurations was determined using step experiments with two different tracers. In one case, the tracer was a solute, while in the second case calcium phosphate was tracked. The obtained RTD curves were plotted in FIG. 31. Differences by reactor type were most pronounced between the TR and the CSTR based precipitation reactors, which exhibited a much broader RTD. Differences by tracer system were negligible for the TR, where the peaks obtained for the solute and the precipitate mostly overlapped. However, the RTD peaks were shifted to higher residence times for the CSTR based reactors, when the step experiment was performed with precipitate (see zoom of RTD plot in FIG. 31B). Usually, a narrow RTD is preferable over a broad RTD. Should disturbances occur in a production process, these would affect a smaller fraction of the process stream if the RTD were narrower. Therefore, the TR would be preferable over the other reactor configurations based on the RTD.

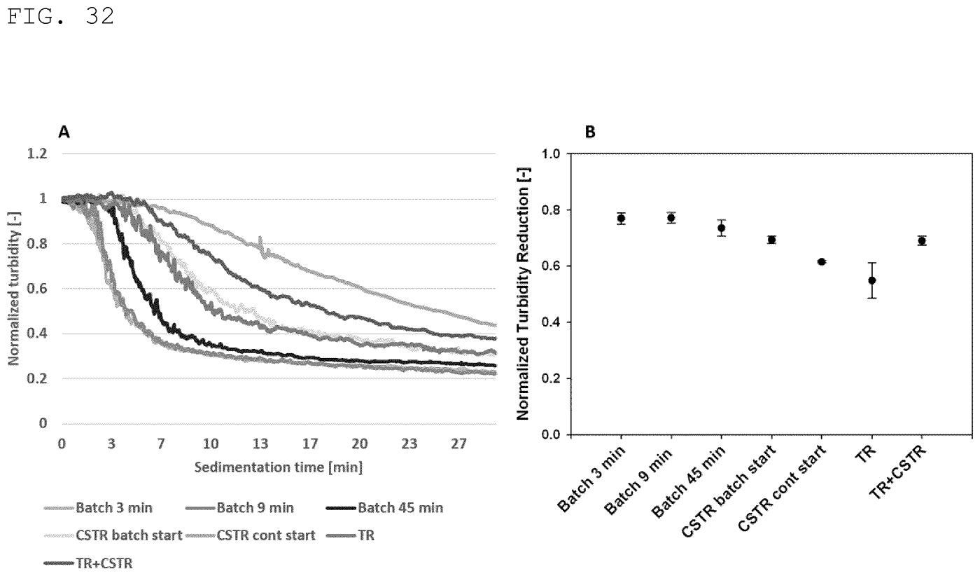

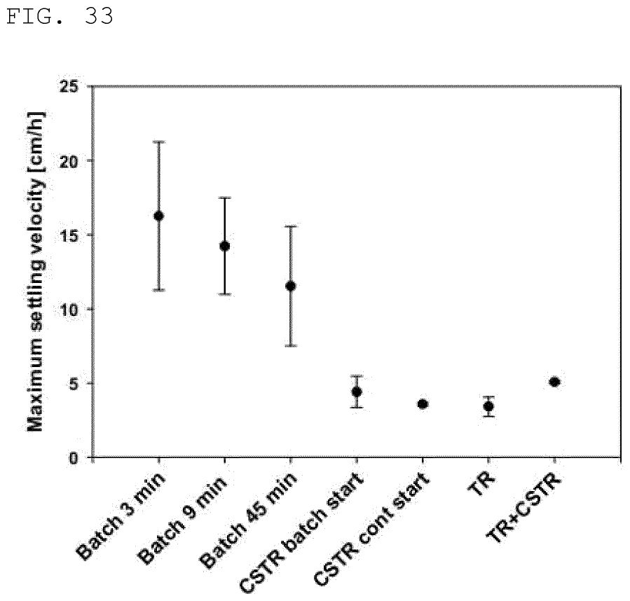

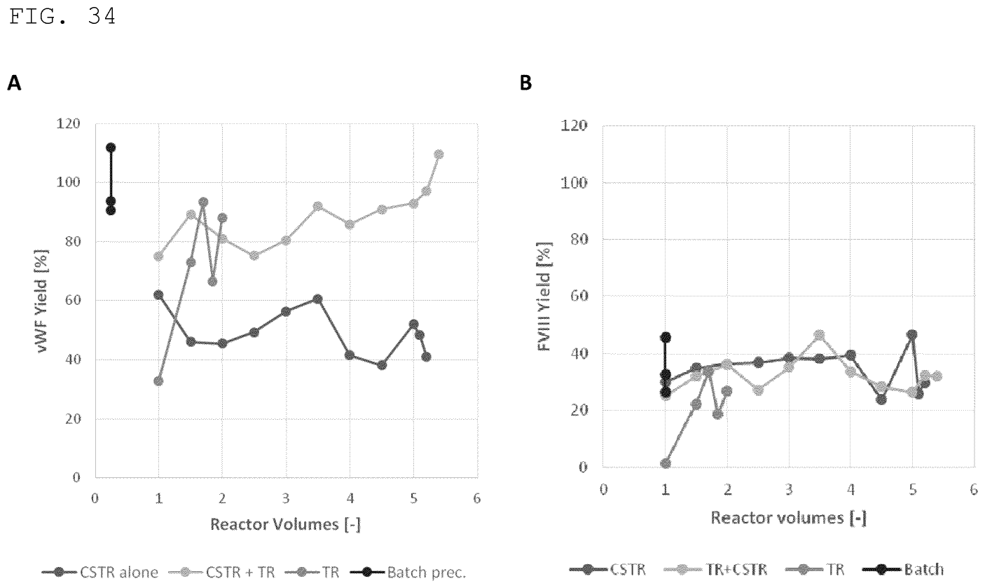

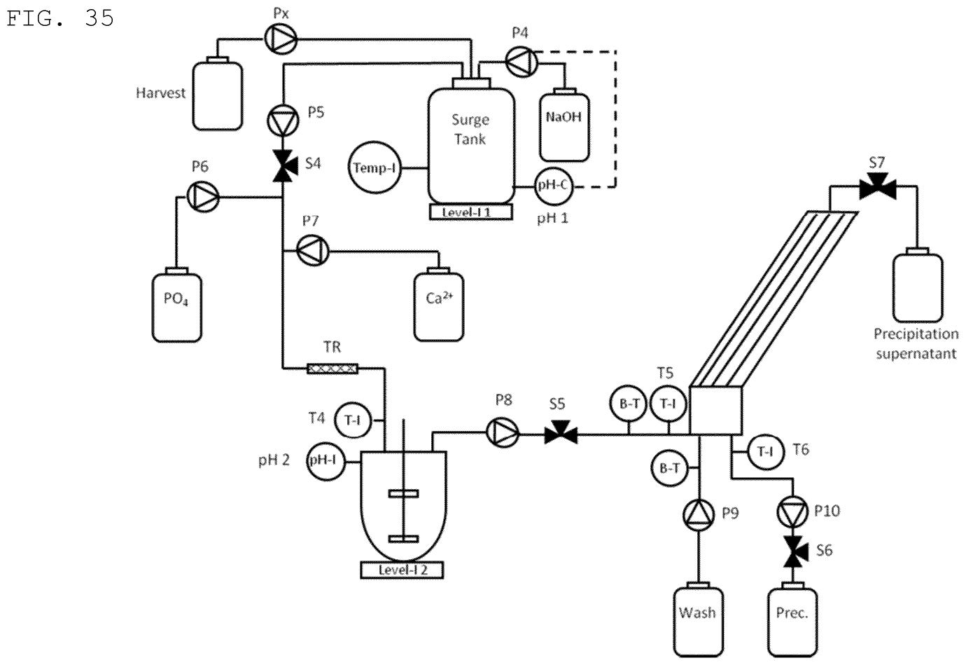

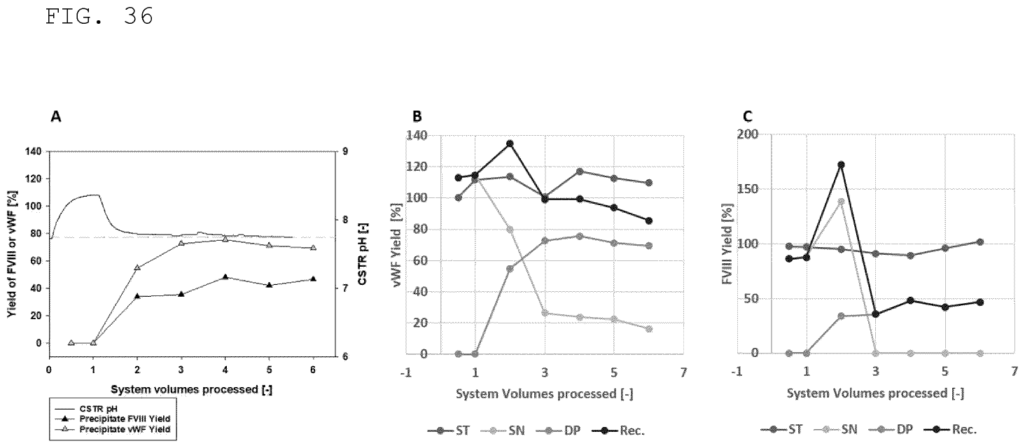

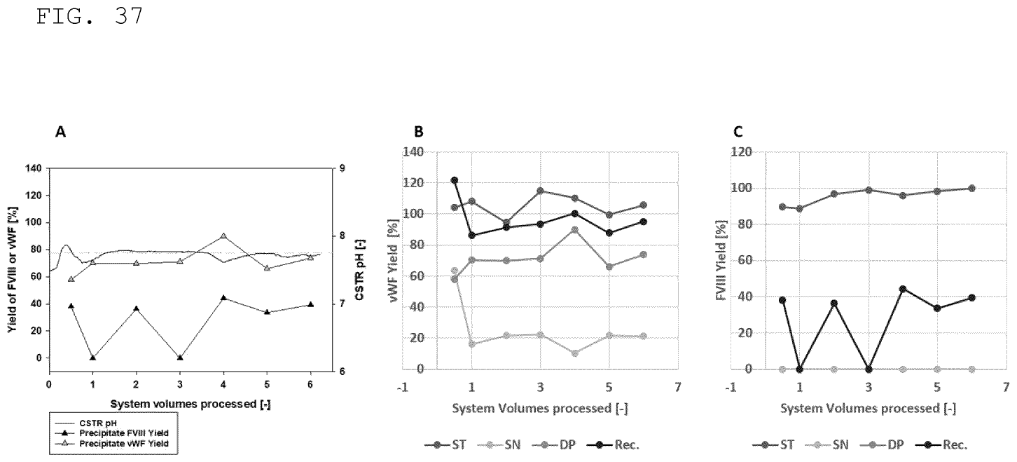

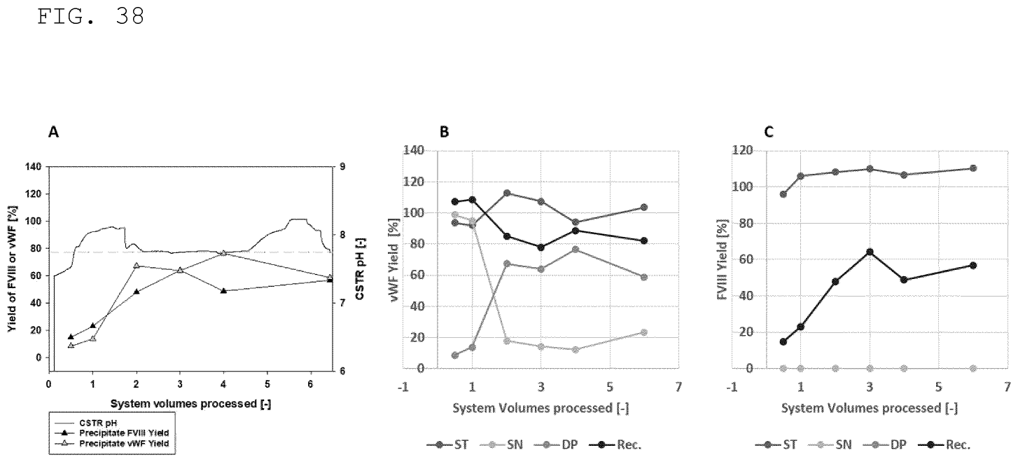

Solid-liquid separation in continuous mode was intended to be performed using an inclined plate settler. The performance of a plate settler is, among other factors, dependent on the settling velocity (SV) of the solid, e.g. calcium phosphate flocks. Earlier observations had indicated that the reactor type might have an influence on the flock size and consequently on the settling behaviour of the flocks (data not shown). Therefore, the reactor types in question for the process were used to produce calcium phosphate precipitate and to determine its settling behaviour. The precipitate settling was monitored using a custom-built sedimentation-monitoring device. The obtained data was interpreted as starting turbidity and turbidity reduction upon sedimentation of the flocks. Turbidity signals were normalized and the average of at least three replicate signals was plotted over time in FIG. 32A. Batch precipitated calcium phosphate exhibited the fastest sedimentation behaviour, followed by the TR+CSTR combination and the CSTR started with a batch precipitation. The precipitate produced in the TR showed the slowest sedimentation. Due to the differences in settling velocity observed, the final turbidity after a given sedimentation time differed. The reduction of the normalized turbidity is shown in FIG. 32B. These points correspond to the inverse of the last points of the curves plotted in FIG. 32A. The higher a turbidity reduction achieved the better.

From the turbidity reduction over time, the sedimentation velocity could be derived. The sedimentation velocity of the slowest settling fraction is what limits a solid liquid separation step. Therefore, the sedimentation velocity of the slowest third (exact: 31.5%) was determined and plotted in FIG. 33. Highest settling velocities were observed for batch-precipitated calcium phosphate, however, these precipitates also exhibited the largest difference between individual replicates. The difference between replicates and between reactor configurations and operation modes for continuously produced precipitate was much smaller. Out of the continuous precipitation reactors, the combination of TR+CSTR produced the fastest settling precipitate followed by the CSTR alone.

Continuous precipitation of the FVIII:VWF complex from thawed CCSN was performed to check for potential differences in product yield between reactor types and batch vs. continuous operation (FIG. 34). Operational stability over time was simultaneously monitored. A reference batch precipitation was performed using the same CCSN starting material and stock solutions as for continuous precipitation. The yield in the batch-precipitated samples was on average 35 and 99% for FVIII and VWF, respectively. The FVIII yield in continuously precipitated samples was comparable with the batch reference, but was significantly lower than what had been previously observed. It was speculated, that the low FVIII yield could be owed to the extended storage duration of the starting material (>1.5 years at −80° C.). VWF yield in the batch reference was in line with previous batch precipitation results and results obtained for continuous precipitation using the TR and TR+CSTR. VWF yield with the CSTR alone was significantly lower. Samples taken over the course of the continuous precipitation experiments, showed stable operation of all reactors. For the TR an initial ramp up phase was observed, which was due to the initial wash out of buffer from the reactor. In summary, there were no differences for FVIII yield. However, there was a difference between the CSTR and all other precipitates for VWF yield. Product yield in the TR and the TR+CSTR was equal.

As for practical considerations regarding the handling during the actual experiments, the CSTR comprising reactor configurations had a significant advantage over the TR. Installation of sensors into an open vessel is simpler than inline installation. Additionally, pH modification can easily be realized with one pH sensor and one control unit in a CSTR. pH adjustment in a TR would require multiple sensors and control units in series. When precipitation was performed in the CSTR, where the origin of particle formation was in the stirred vessel, extensive sensor fouling was observed.

Based on the data describing the reactor's RTD, sedimentation of precipitates from these reactors, product yield and considerations with regard to the practical realization and handling, points were awarded in the predefined categories (see Table 19). The combination of TR+CSTR performed best based in this point based evaluation method. The configuration including TR+CSTR was therefore selected for implementation in the setup for continuous precipitation of FVIII:VWF in an integrated setup. However, later experiment indicated that the precipitate may stall and accumulate in the TR (see subsequent section), which suggests that it may be advantageous to remove or replace the TR with another mixing device.

TABLE 19
Decision matrix for selection of a reactor for continuous precipitation
of calcium phosphate with points awarded in the individual categories
and sum of awarded points for each reactor.

| | Reactor type | | |
|---|---|---|---|
| Category | CSTR | TR + CSTR | TR |
| RTD | 1 | 0 | 2 |
| Settling velocity | 1 | 2 | 0 |
| Protein yield | 0 | 1.5 | 1.5 |
| Practical realization | 1 | 2 | 0 |
| Sum of the above | 3 | 5.5 | 3.5 |

### Automated, Continuous Precipitation of FVIII:VWF at Lab-Scale

Continuous precipitation of FVIII:VWF from CCSN was performed using the automated, continuous prototype equipment, which is schematically depicted in FIG. 35. The equipment was first tested with protein free buffer, during which the pH control parameter values were established and the software was tested while the final software version was being programmed. The values for pH control were confirmed with CCSN (data not shown). Experiment 1 (FIG. 36), which lasted for approx. 4.5 to 5 h each or a total of six system volumes processed, was performed in triplicate. The results obtained for yield of VWF and FVIII in the surge tank, where the pH was modified, were highly stable around 100%. Yield in the precipitation step per se was dependent on the pH value in the CSTR. When the CSTR pH was stable, so were the product yields. Disturbances in the CSTR pH were observed, when too little or too much of either of the precipitant stock solutions were dosed. An excess in precipitants, caused a decrease in pH, while a lack resulted in an increase in pH. The majority of the disturbances observed in the continuous precipitation runs were operator induced. The system with its underlying control mechanisms was operating as expected. At steady state, the pH value in the CSTR was 7.75±0.1. In all experiments (FIGS. 36-38), even under stable precipitation conditions, the yields of VWF and FVIII were lower than what had previously been observed in comparable batch experiments. Precipitation of VWF appeared to be incomplete, with residual VWF present in the precipitation supernatant. For FVIII the mass balance was incomplete, with roughly 50% of FVIII remaining unaccounted for after summing up the relevant fractions. The FVIII analysis performed was an activity assay, which means the protein might still be present, but inactive. This would explain the apparent loss of FVIII.

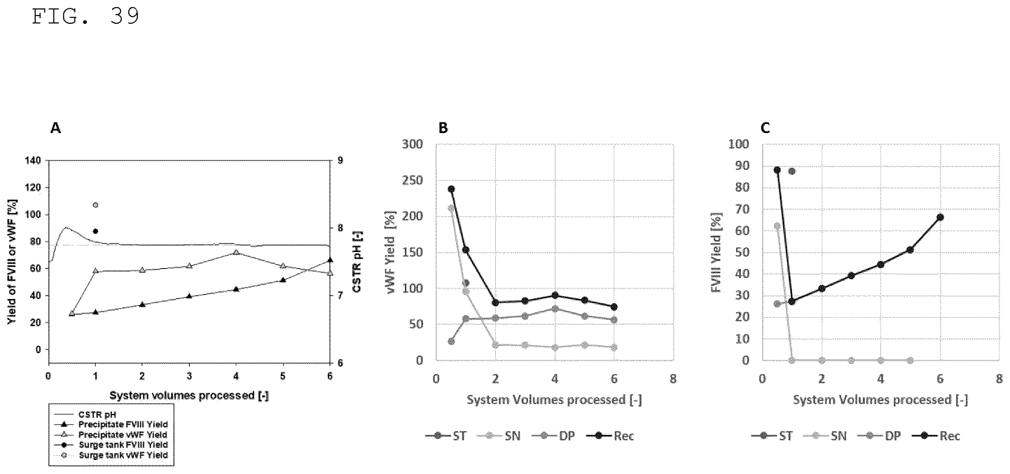

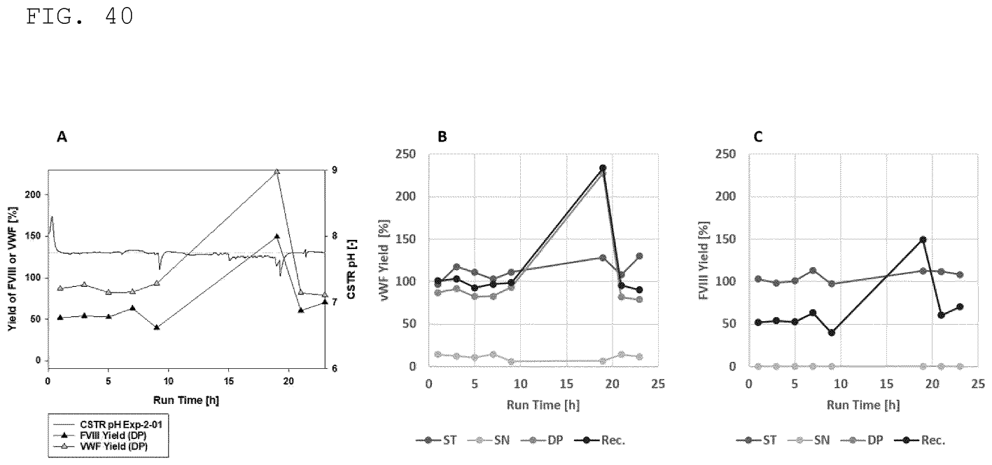

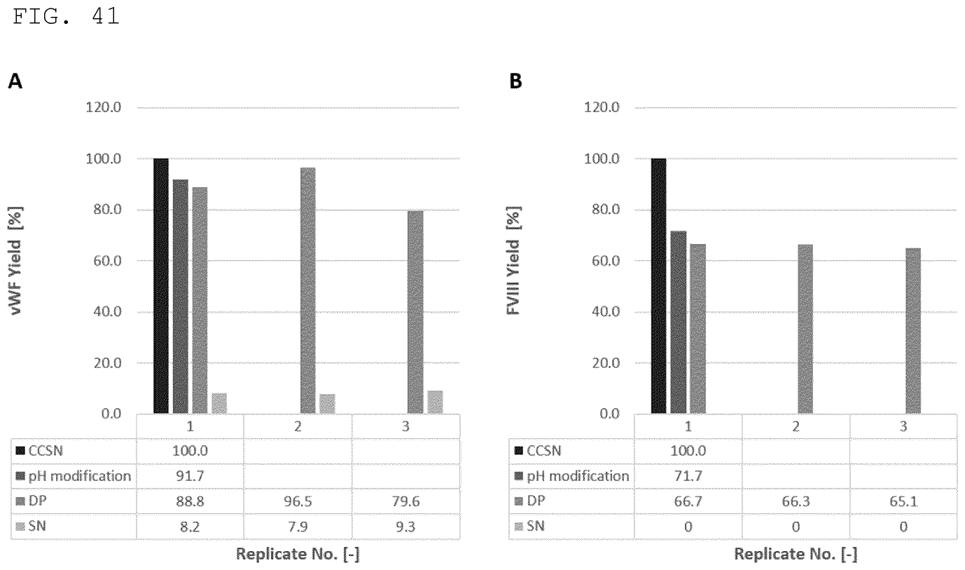

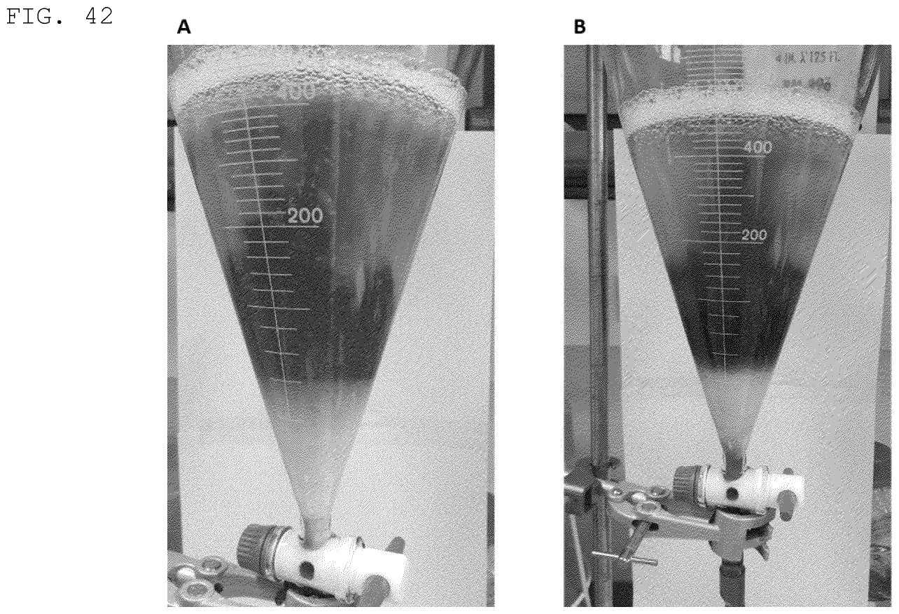

In one experiment, the influence of the tubular reactor on the continuous precipitation was investigated by removing the tubular reactor from the setup (FIG. 39). The precipitant stocks were dosed in the same manner and the precipitate suspension was transported to the CSTR via an open tube. In this experiment no disturbances were encountered. The initial increase in CSTR pH (see FIG. 39A) was due to mixing of the unmodified CCSN with the pH modified CCSN from the surge tank. The pH increased until it reached a value at which calcium phosphate precipitation had increased high enough to lower the pH to its steady state level. The stability of the CSTR pH over the course of the experiment was reflected in the yield of VWF. For FVIII an increase in yield with increasing system volumes processed was observed.

Feasibility of operating the continuous precipitation over prolonged periods of time was shown by runs lasting for ˜24 h. The results obtained in an experiment with the precipitation alone are shown in FIG. 40. Overall, the automated control resulted in stable conditions over the entire run time of the experiment. The range between 10 to 18 h corresponded to the time, during which the system was unsupervised. The CSTR pH remained stable within this period. The spike in product yield at roughly 18 h was caused by manually displacing stalled precipitate from the tubular reactor. The sample collected thereafter, contained a higher amount of precipitate. The yield in this case was above 100% as the material collected in this sample corresponded to material that had accumulated over time. These results speak to removing or replacing the tubular reactor with another unit operation or mixing device. Ideally, the alternative to the TR would not cause stalling of the precipitate.

Fresh CCSN was precipitated in batch as a control for the continuous precipitation performed in the automated setup. The batch precipitate showed average yield values of 88% and 66% for VWF and FVIII, respectively (see FIG. 41). In the batch samples, the precipitation of VWF was incomplete with 8.4% of VWF remaining in the precipitation supernatant. This had been observed in the continuous runs, but had not been observed in any prior batch experiments. Notably, batch experiments for precipitation condition determination had always been performed with thawed CCSN, while the continuous experiments were performed with fresh CCSN (i.e. without a freeze-thaw cycle). It is possible that the carbonate buffer system in the CCSN is affected by the freeze-thaw cycle. Such a difference in the buffer system of the CCSN could have an influence on the calcium phosphate precipitation, thus causing the reduction in product yield observed in the continuous runs and the batch reference sample.

### Example 9: Continuous Precipitate Collection Using an Inclined Plate Settler

Continuous solid-liquid separation represents one of the bottlenecks in continuous processing. A new concept for the bottom section of inclined plate settlers is disclosed herein, and exemplified by Examples 1 to 6. This Example 9 demonstrates continuous precipitate formation and collection using this new bottom section/inclined plate settler in accordance with the present disclosure.

### Wash Buffer Composition for Use in Inclined Plate Settlers

The new bottom section/inclined plate settler in accordance with the present disclosure enables the implementation of a wash step. In order for the solids to settle into the wash buffer without extensive mixing with the original liquid phase, the densities need to be in the following order:

ρ_{solids}>ρ_{wash}>ρ_{liquid} (Equation 4)

Depending the concentration of solute(s) a solution's density will change. For the separation of calcium phosphate precipitate the density of the solids and the surrounding liquid was given. Therefore, the wash buffer composition had to be chosen such that it would fit the requirement according to Equation 4.

First, settling experiments were performed using protein-free precipitate suspension and buffers containing different NaCl concentrations for density modification. The tested compositions were adapted from theoretical densities calculated based on multiple density measurements (data not shown). The experiments were deemed successful if settling of the flocks into the wash buffer was achieved without extensive mixing of the wash buffer with precipitate's mother liquor. In other words: if there were still three separate phases distinguishable at the end of the settling experiment. For the precipitate formed in protein-free buffer, this was achieved at a concentration of 270 mM NaCl.

Precipitate produced in cell culture supernatant was settled in the same manner. Here, buffers with and without CaCl₂ supplementation to 12 mM were tested. When 15 mM calcium were added in the precipitation step, the residual calcium concentration in the precipitation supernatant determined by calcium colorimetric assay was around 12 mM. This calcium concentration was chosen as the maximum concentration to be supplemented. Wash buffer without calcium represented the minimum supplementation. The density requirement was fulfilled and settling into the wash buffer proven to be feasible at NaCl concentrations of 272 and 231 mM with 0 and 12 mM CaCl₂), respectively. Pictures taken at the end of these settling experiments are shown in FIG. 42.

From these experimental results a trade-off between CaCl₂ and NaCl could be calculated with combinations of NaCl and CaCl₂ on the line in FIG. 43 assumed to result in solutions of equal density. All of the resulting solutions should therefore be suitable as wash buffers.

### Wash Buffer Calcium Concentration for Maximizing Product Yield

The goal of the wash step was a reduction of the liquid phase calcium concentration in order to increase the efficiency of the following dissolution. To that end, a calcium concentration lower than in the precipitation supernatant (i.e. 12 mM) was required. Four different wash buffers were tested for their influence on product yield with calcium concentration at 0, 4, 8 and 12 mM. The yield in these samples was compared to a reference sample that was settled in the same manner, but not washed. The yield in these experiments was lower than in previous experiments with thawed CCSN (compare FIG. 44A). In the separation funnel, it was not possible to collect all of the precipitate present. When the precipitate suspension was collected through the bottom outlet of the funnel, precipitate flocks adjacent to the funnel's walls remained stationary. In early batch precipitation experiments, a strong correlation between calcium phosphate precipitate and product yield had been obtained. The loss in precipitate observed in the separation funnel experiments therefore explained the decreased yield. Therefore, the yield in the reference sample corresponded to the maximum yield obtainable at any wash buffer condition. The washed samples were compared to this reference sample in FIG. 44B. The results indicated a minimum of 4 mM CaCl₂ had to be supplemented to the wash buffer in order to achieve stable product yield. Including a safety factor, a wash buffer consisting of 2 mM TRIS, 6 mM CaCl₂, 252 mM NaCl, pH 7.75 at RT was chosen for solid-liquid separation of calcium phosphate precipitate in an inclined plate settler.

### Optimization of Inclined Plate Settler Operation Parameters for Calcium Phosphate Collection

The prototype single plate inclined settler was designed for solid-liquid separation of the precipitate produced in the continuous precipitation step (i.e. continuous capture). It was equipped with a bottom section employing the newly developed concept of separate inlet channel(s), collection channel(s) and wash fluid supply channel(s) disclosed herein. Discharge of the solids was performed periodically by the action of two simultaneously operated pumps. One pump (sludge pump) withdrew the concentrated suspension (sludge), while a second pump supplied wash buffer at the same flow rate (wash pump) in the first part of the discharge cycle. In the second part of the discharge cycle, the sludge pump was operated alone to lower the wash buffer level in the bottom section to the original level. This was necessary, because the precipitate displaced the wash buffer pushing it upwards. In the end, the precipitate was transferred from the mother liquor into the wash buffer, ideally without any mixing between the two “phases”. However, some mixing was expected with the extent being dependent on the discharge flow rate. Three flow rates were tested with two different tracers to check for mixing between the wash buffer and the feed stream. The concentrations of the tracers were quantified in the discharged fractions. The data point with 77% yield in FIG. 45A corresponds to the very first discharge in the experiment. Most likely, the bottom section still contained some feed buffer as a remnant from the system start-up. In all other data points, little mixing with yields above 89% and below 11% was observed when the tracer was in the wash buffer or in the feed stream, respectively. In the ideal case of no mixing, the yield in the discharge fraction would be expected to be 100% if the tracer was supplemented to the wash buffer and 0% if the tracer was supplemented to the feed stream. In panel A of FIG. 45 an increase in mixing was observed with increased flow rate. In panel B mixing first increased and then decreased again with increasing discharge flow rate.

In addition to the evaluation of mixing based on the tracers, the discharge peaks obtained at the different flow rates were compared. At the lowest flow rate the peaks exhibited significant tailing (FIG. 46A), which was abolished once the flow rate was increased to 40 mL/min (FIG. 46B). When the flow rate was increased even further, the peaks appeared broader than before (FIG. 46C). The intermediate flow rate seemed to balance efficient discharge without tailing and little mixing. Based on these results the following experiments were performed using a discharge flow rate of 40 mL/min.

The results above were obtained with a discharge interval of 15 minutes. During every discharge, a given volume of wash buffer was consumed in order to replace the precipitate mother liquor. Therefore, shorter discharge intervals resulted in higher wash buffer consumptions. Longer discharge intervals, which corresponded to lower buffer consumption, were tested (FIG. 47). During these experiments, the concentration of Patent Blue V was quantified in the top overflow. The yield in the top flow fractions was zero. Yield in the discharge fractions was highest with the shortest discharge interval and decreased with increasing interval length. The wide range over which the yield scattered for the 45 min discharge interval was due to a disturbance: During these discharges, the system ran out of wash buffer and therefore the discharges were diluted by precipitate suspension. The overall trend indicated increased mixing between the wash buffer and the feed stream with increasing interval length. Even though the density difference between the solutions should prevent extensive mixing, over longer periods of time diffusion effects may become significant. The interval length should be chosen such that it balances buffer consumption, mixing by diffusion and fill level of the bottom section. The solid load per discharge increased with increasing discharge interval length. Increasing solid load caused a slight increase in the discharge peak maximum, but mostly resulted in broader discharge peaks (see FIG. 48). However, within the tested discharge interval range, the bottom section was not overloaded yet. The 30 min interval was chosen in order to prevent an excess of mixing due to diffusion and for practical reasons with regard to the experiment duration for testing a given number of conditions at a constant discharge interval.

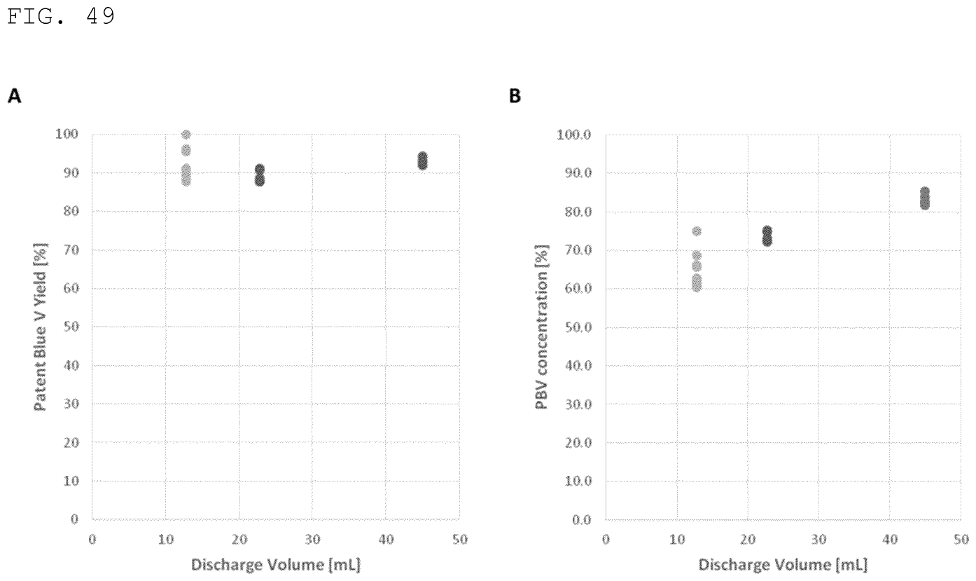

The volume reduction in the precipitation step was highly dependent on the efficiency of the solid-liquid separation step. The peaks obtained with a discharge interval of 30 min (see FIG. 48A) indicated, that the majority of the solids was discharged within the first 25 mL of the discharge. In order to increase the volume reduction, the discharge peak had to be cut earlier, which meant reducing the discharge volume. The discharge volume was first reduced to roughly half and then to a quarter of the original discharge volume. Under all conditions, high yield of the tracer was obtained in the discharges (FIG. 49A). In contrast to yield, the concentration of Patent Blue V in the discharge fraction decreased with decreasing discharge volume (see FIG. 49B). This was due to the way the discharge was operated. The fraction of the discharge volume, produced by sludge flow only, increased with decreasing total discharge volume. The sludge-flow-only-volume (4 mL) was held constant, because it was dependent on the amount of precipitate settled. The amount of precipitate settled was assumed constant for a constant discharge interval.

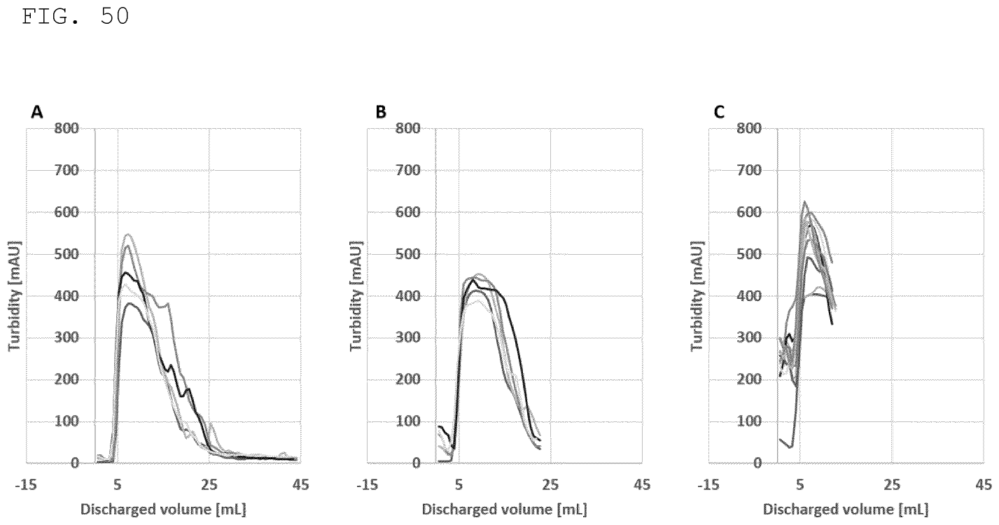

The overlay of the discharge peaks for the tested discharge volumes showed the highest volume reduction of precipitate volume per total discharged volume for the smallest discharge volume (FIG. 50C). Considering the feed volume processed within one interval (30 min×3.5 mL/min) and the discharge volume (12.8 mL), one would end up with a volume reduction by a factor of 8.2. The goal was to have a volume reduction of at least 10 after dissolution of the precipitate, which would require further reduction of the discharge volume. However, the current results showed lower tracer concentration in the smaller volume discharges caused by the introduction of a constant volume of mother liquor. Thereby, the mother liquor depletion from the harvested precipitate suspension would be decreased. The effect of a given mother liquor depletion on the dissolution efficiency was discussed above. Calculations of the calcium contribution of the different fractions (mother liquor, wash buffer, precipitate) have shown the main contribution to be due to the precipitate itself (in this range of volume reduction). Therefore, the importance of efficient mother liquor depletion became secondary.

Continuous Collection of FVIII:VWF Precipitated from CCSN

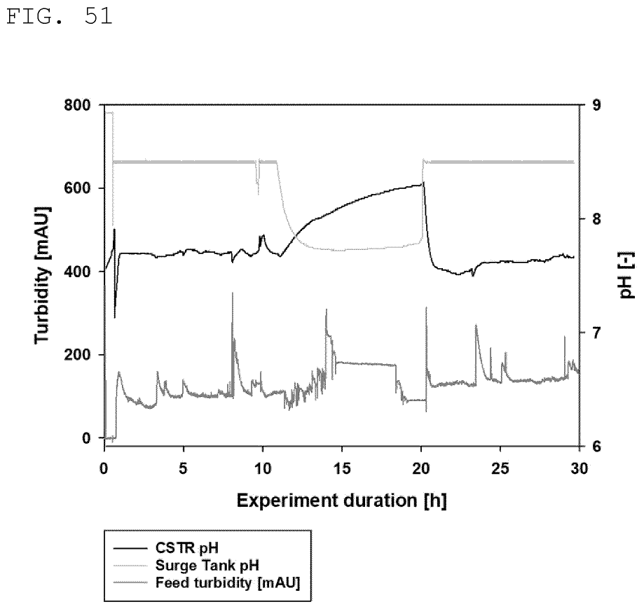

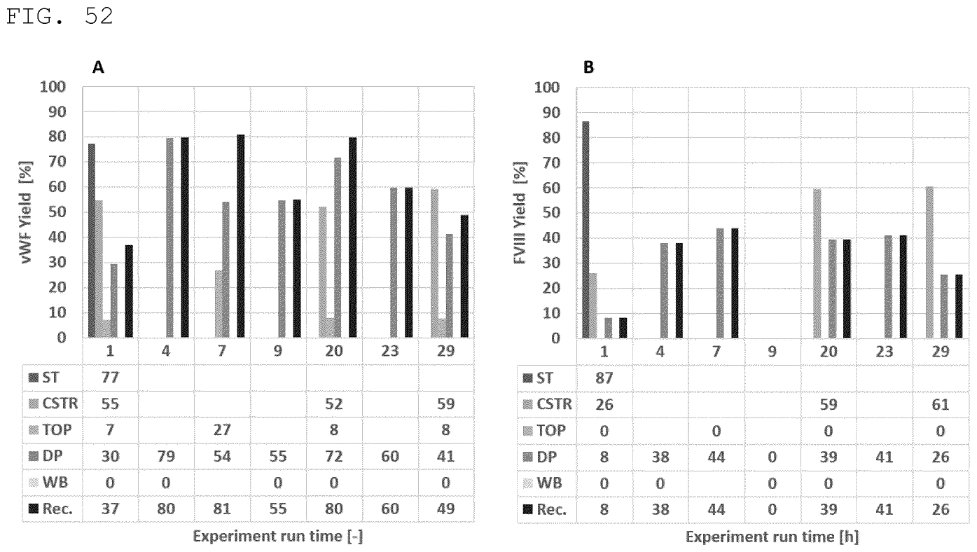

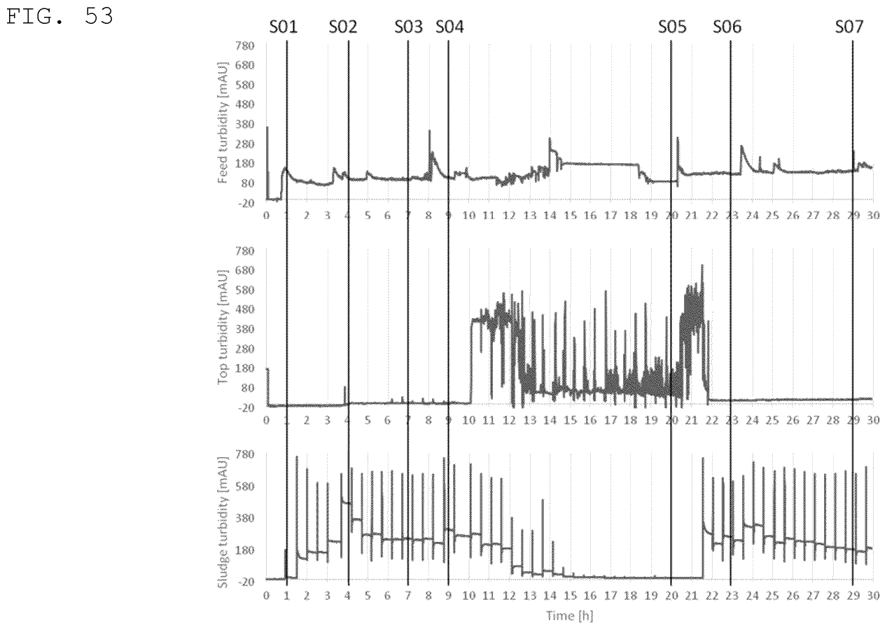

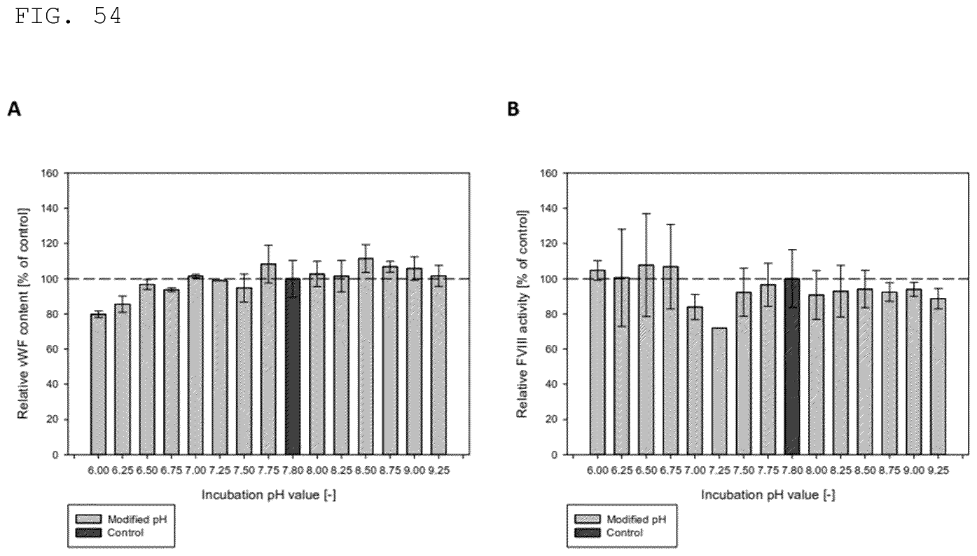

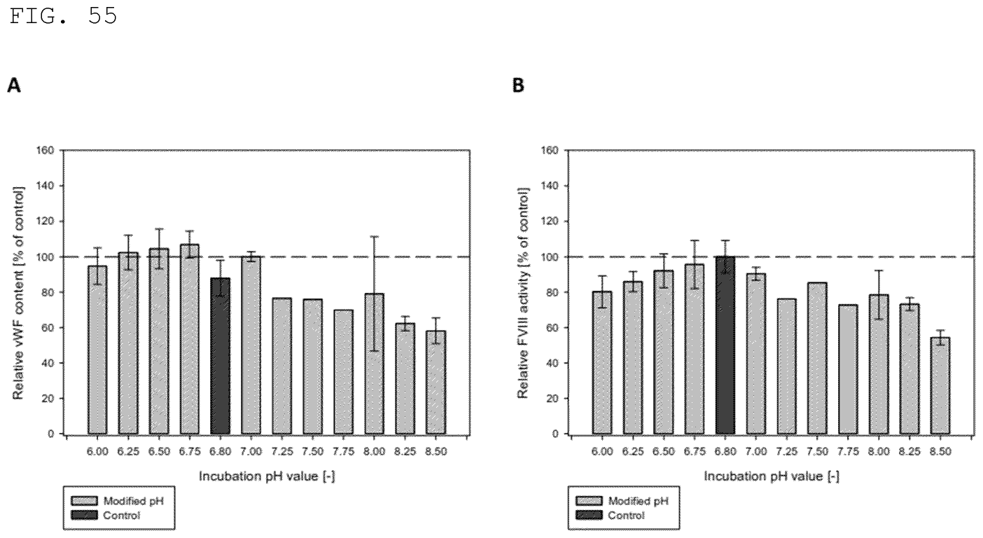

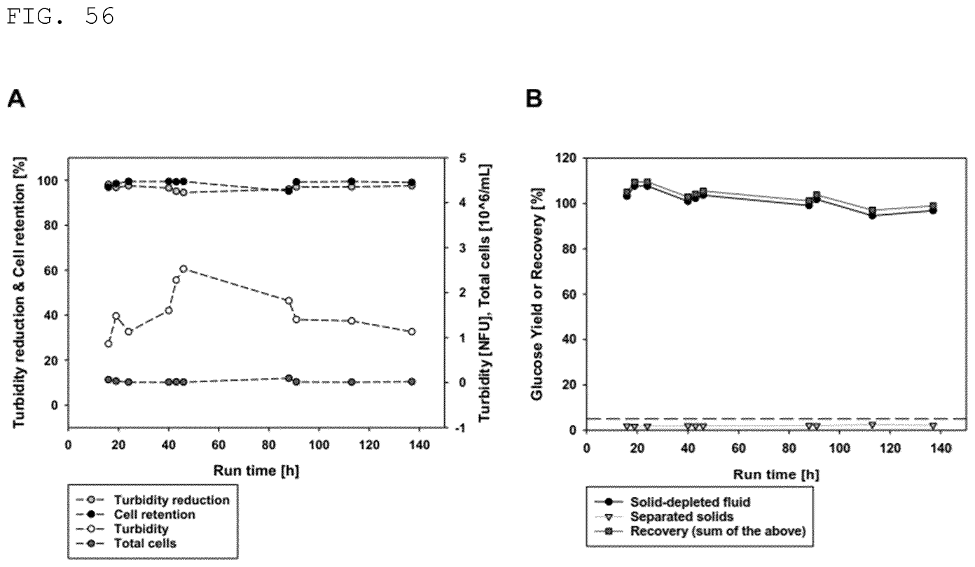

The prototype inclined plate settler was used to collect precipitate formed in CCSN during a continuous precipitation experiment using the lab-scale prototype precipitation setup (FIG. 52). The last pump collecting precipitate from the CSTR in the precipitation, was feeding directly into the inclined settler. The settler was operated at the discharge flow rates, intervals and volume as previously established. The discharge flow rate was 40 mL/min, the interval was set to 30 min and the discharge volume was 12.8 mL per cycle. In this run, there was a major disturbance in the precipitation, during which dosing of NaOH for pH control failed due to an operator error. Therefore, the pH in the surge tank dropped below the critical level and all following steps in the precipitation were put on hold automatically. Consequently, the feed stream into the settler was interrupted. The drop in the pH value in the surge tank and subsequent increase in pH in the CSTR could be observed in the data recorded during the run (see FIG. 51). The feed turbidity signal was noisy. However, on average it remained constant throughout the experiment, which supported stable system operation. When the feed stream to the settler was interrupted, the amount of precipitate discharged per cycle decreased and eventually, all the precipitate had been removed (see FIG. 53, lowest panel). Settler steady state operation could be restored after the precipitation had been restarted. Steady state operation presented a stable turbidity signal at the sludge sensor in between discharges. During the disturbance in the precipitation and the ensuing lack of feed stream to the settler, the top turbidity sensor had drawn air. Therefore, an increased turbidity level at the top sensor was observed during that period (see FIG. 53, middle panel).

The yield obtained in the integrated run was comparable to what had been observed in the experiments with the continuous precipitation without continuous solid liquid separation. What had previously been labelled SN (precipitation supernatant) was represented by the TOP fraction in this experiment. Protein (VWF) that remained in solution despite the precipitation step, was found in this fraction. The precipitate was transferred from the precipitation supernatant to the wash buffer. In the wash buffer samples, neither VWF nor FVIII were detected. Based on this data, it seemed the calcium phosphate flocks were stable in the wash buffer and did not release any of the captured product. For FVIII, yield in the dissolved precipitate discharged from the settler, was lower than in the dissolved precipitate collected before the settler. This could be due to the wash buffer matrix after dissolution. The wash buffer did not contain any surfactants, which were still present as long as the matrix consisted mainly of CCSN.

### Example 10: Precipitation Complementing Data

With regard to precipitation of the FVIII:VWF complex by calcium phosphate precipitation, aspects relevant to the precipitation were investigated. Stability of the product molecules at the process pH was an important prerequisite for the precipitation. For dissolution, the highest possible stock concentration of citrate was investigated. Furthermore, the ratio at which calcium was complexed by citrate was determined experimentally. Knowledge of the complexation ratio allowed estimating the theoretical citrate demand for dissolution and comparison with the actual demand observed.

### pH Stability of FVIII and VWF

Precipitation of the FVIII:VWF complex from CCSN included a pH modification step, as described for batch precipitation in “Calcium phosphate batch precipitation”, for kinetics in “Calcium phosphate batch precipitation—Precipitation kinetic studies” and for continuous precipitation in “Continuous precipitation of calcium phosphate” and “Automated, continuous precipitation of calcium phosphate—pH control”. To determine the pH region, within which FVIII and VWF were stable, samples were titrated to different pH values below and above the pH of CCSN. Stability was evaluated as concentration relative to a CCSN sample. When the FVIII:VWF complex was intact, both FVIII and VWF exhibited high stability across the tested pH range. FVIII stability decreased slightly at alkaline pH values, whereas in the complex a decrease in VWF stability was observed below pH 6.5 (see Panel A and B, respectively in FIG. 54). The addition of the salt stock caused the complex to dissociate. Stability towards changes in the solution pH was reduced for both molecules. FVIII and VWF were more labile at alkaline pH values. Yield in these samples was around 60% of the control sample (FIGS. 55C and D).

### Examples 7 to 10: Conclusions

The following conclusions are drawn from the above Examples 7 to 10.

A continuous capture step for the recombinant FVIII:VWF complex was developed. FVIII is a highly labile molecule, which is co-expressed with rVWF for stabilization. Nevertheless, the product remains comparably labile and is therefore produced in continuous manner in a stirred tank reactor. In order to reduce manufacturing costs continuous downstream processing was identified as the primary target. As capture step a continuous precipitation was proposed. Batch precipitation experiments were conducted to optimize the precipitation conditions, which were transferred to continuous operation. The batch precipitation process showed product yields of up to 92% for FVIII and 99% for VWF. Host cell protein (HCP) was reduced by 70% and 21% of dsDNA could be removed. The capture step resulted in an eightfold volume reduction.

Proof-of-concept of fully automated continuous processing was demonstrated using prototype equipment. The continuous precipitation was operated at steady state for 23 hours, after which the experiment was willfully terminated. Operator action was only required for process supervision and sample handling. Any disturbances observed were operator induced. Furthermore, stability and reproducibility were supported by replicates of shorter continuous precipitation experiments. These results demonstrate the advantages of precipitation in process development, when aiming at continuous operation, which is simplicity. The integrated continuous runs are proof-of-concept for the feasibility of establishing a precipitation based continuous capture step.

Solid-liquid separation in continuous mode was performed using an inclined plate settler with a recently developed bottom section concept of separate inlet channel(s), collection channel(s) and wash fluid supply channel(s) disclosed herein. The concept employed allows the implementation of a wash step and provides homogenous flow distribution onto the individual plates of the settler. In this work, a prototype inclined plate settler designed for the collection of calcium phosphate precipitate from CCSN was used. Operation conditions were determined with protein free precipitate and were shown to be directly applicable to precipitate formed in cell culture supernatant. The continuous precipitation and continuous solid-liquid separation were successfully integrated. They were operated in a fully automated fashion for >24 h. During this time an unintended, operator induced disturbance occurred. After the disturbance had been resolved, the process returned to steady state operation without any further intervention. This demonstrates the robustness of the process as such and its capability to recover after a disturbance. The fact that processes can recover and a disturbance does not result in full batch failure is one of the main benefits of continuous processing.

It has recently been reported that solid-liquid is one of the bottlenecks in continuous downstream processing (Reference 6). The results presented here highlight the advances in solid-liquid separation by inclined plate settlers with the recently developed bottom section concept. The concept allows continuous solid-liquid separation including precipitate wash without compromising flock structure. Precipitate compaction, thus destroying native flocks, was reported to hamper dissolution in PEG precipitation. By providing means to de-bottleneck solid-liquid separation, inclined plate settlers could also be an enabling technology for a larger number of precipitation processes in continuous downstream processing. Precipitation enables a reduction in process volume from which all subsequent unit operations can benefit. The results are shorter processing times, e.g. column loading times, and lower buffer demand, e.g. for conditioning steps, and thereby increased process efficiency.

Continuous processes are ideally suited for automation, because individual steps are intrinsically integrated, which means physically connected. On the contrary, individual unit operations in batch processes are separated, which would require an additional effort for integration and automation. Automation offers a higher degree of product consistency by removing process variability associated with operator action. Furthermore, labor costs in production can be significantly reduced. Fully automated processes also require less floor space, because human intervention is not necessary. Therefore, cost of goods is significantly reduced for a continuous, fully automated process.

### Examples 11-13: Materials and Methods

The following materials and methods were used in the subsequent Examples 11 to 13: Materials

### Cell Culture

Fermentation of CHO cells was performed in a 10 L bench-scale bioreactor in chemostat mode. Cells were cultured using SPRA medium at a cell density of 1.5×10\hat{ }6 cells/mL. During one campaign FBA medium supplemented with Glutamine was used instead of the standard SPRA medium. By transiently reducing the dilution rate, cell density was increased first to 3×10\hat{ }6 cells/mL and finally to 6×10\hat{ }6 cells/mL.

### Cell Culture Supernatant

CHO cell culture supernatant containing recombinant FVIII and recombinant VWF for use in precipitation experiments was collected for 24 h before clarification by depth and membrane filtration. The clarified cell culture supernatant was shipped on wet ice, and subsequently aliquoted and stored <−60° C. until further processing. Aliquots were thawed overnight in a water bath at 2-8° C. before each experiment. Alternatively, aliquots were thawed at 37° C. in a water bath on the morning of an experiment.

### Chemicals and Stock Solutions

A solution of 0.25 M NaOH was prepared by dilution from 10 M NaOH (Merck, 480648). Calcium and phosphate stock solutions were prepared by dissolution of the respective chemicals in water. Calcium (CaCl₂*2 H2O, Sigma, C5080) stock concentration was 4 M. Phosphate stock concentration was 0.2 M (Na₂HPO₄, Merck, 106580). Citrate stock (1 M) was prepared by dissolution of citric acid (citric acid monohydrate, Merck, 100244) in water and subsequent titration with solid NaOH (Merck, 106482) to pH 7.0. TBS-T buffer with a final concentration of 3.152 g/L TRIS-HCl (Sigma, T5941), 0.6055 g/L TRIS-base (Merck, 108382), 8.766 g/L NaCl (Merck, 106404), 0.50 mL/L Tween 20 (Sigma, P9416) was used. The concentration of EDTA (Merck, 1084211000) was 0.1 M in water. PBS-C (8 g/L NaCl, 0.2 g/L KH₂PO₄, 1.15 g/LNa₂HPO₄, pH 7.0) was supplemented with either 6 or 9 g/L NaCl and used for cell removal with in inclined plate settler.

### Inclined Plate Settler

An inclined plate settler prototype was used. The prototype was comprised of a sedimentation section with four sedimentation channels. The sedimentation section was in assembly with a bottom section by which each sedimentation channel was individually supplied with feed fluid and the solids descending from each sedimentation channel were separately collected in a separate collection channel in the bottom section. Thus the bottom section used in these experiments represents a quadruplicated version of what was used in examples 7 to 10. The settling section was made from acrylic glass and was comprised of four settling channels. The individual settling channels were separated by 2 mm acrylic glass plates. This settling section was either combined with a structured bottom section with a flow distributor system for each individual settling channel or with a conventional bottom section known in the art, where all settling channels are supplied via an open space at the bottom of the settling section (in the following also referred to as a “conventional bottom section”).

### Methods

### Inclined Plate Settler Operation

The inclined plate settler prototype was operated at a constant feed flow rate of 3.5 mL/min. Operation was controlled by the custom software tool programmed in National Instruments LabVIEW. For the structured bottom section, discharges were performed at an interval of 60 min with simultaneous flow of 60 mL/min of the wash fluid and the discharge pump. The discharge volume was held constant at approx. 38 mL/discharge. For the conventional bottom section, the discharge interval was 30 min, upon which around 12 mL of solid suspension were collected in every discharge cycle at a flow of ˜12 mL/min of the discharge pump alone.

### Precipitation of Cell Culture Supernatant

Cell culture supernatant was homogenized and the pH value was adjusted to pH 8.5 or 8.75 using 0.25 M NaOH at 2-8° C. Under constant mixing at approx. 150 rpm, phosphate and calcium stock solutions were added to a final concentration of 2 and 15 mM, respectively. Depending on the experimental setup, the precipitate suspension was divided into smaller aliquots. Samples were taken from the unmodified cell culture supernatant and after pH adjustment.

### Solid-Liquid Separation

Separation of the precipitate from the precipitation supernatant was performed by gravity settling for at least 3 h with subsequent centrifugation at 5000 g, 10 min, 4° C. Centrifugation was performed using a Heraeus Multifuge X3 FR swing-out rotor centrifuge (Thermo Scientific) at 4° C. For the reproducibility study centrifugation speed and duration were either 5 min at 4800 or 1000 g, 4° C. After solid-liquid separation the precipitation supernatant was sampled and subsequently discarded. The collected precipitate was re-solubilised after re-suspension. in 3 mL TBS-T buffer after centrifugation.

### Re-Solubilization of Separated Precipitate

The standard approach for re-solubilisation was addition of 1 M citrate, which was done in a step-wise manner until complete dissolution or as a single. Alternatively, 0.1 M EDTA was used in the same manner. The re-solubilised precipitate was sampled.

### Product Analytics

FVIII concentration was measured using an activity based 96-well plate format assay. The assay was based on the SP4 FVIII chromogenic kit (Coachrom Diagnostica, 82 4094 63). Quantification was performed based on a 2nd degree polynomial standard curve using FVIII reference material. A second sample of FVIII reference material was used as an internal control. For selected samples VWF concentration was determined using VWF antigen ELISA. VWF concentration in the samples was quantified using a linear calibration approach based on VWF reference material and internal control.

### Example 11: Cell Removal Using an Inclined Settler with a Structured Bottom Section

In some embodiments, the present invention includes a cell separation step of separating cells from the fluid comprising the protein in accordance with the invention. Preferably, this cell separation is performed using a plate settler for cell separation that is connected to a bottom section in accordance with the invention. This example demonstrates that such cell separation using the newly developed plate settler/bottom section (in the following also referred to as an inclined settler with a structured bottom section) is advantageous compared to cell separation using a conventional plate settler/bottom section (in the following also referred to as an inclined plate settler with a conventional bottom section).

Cell Removal Using an Inclined Settler with a Structured Bottom Section

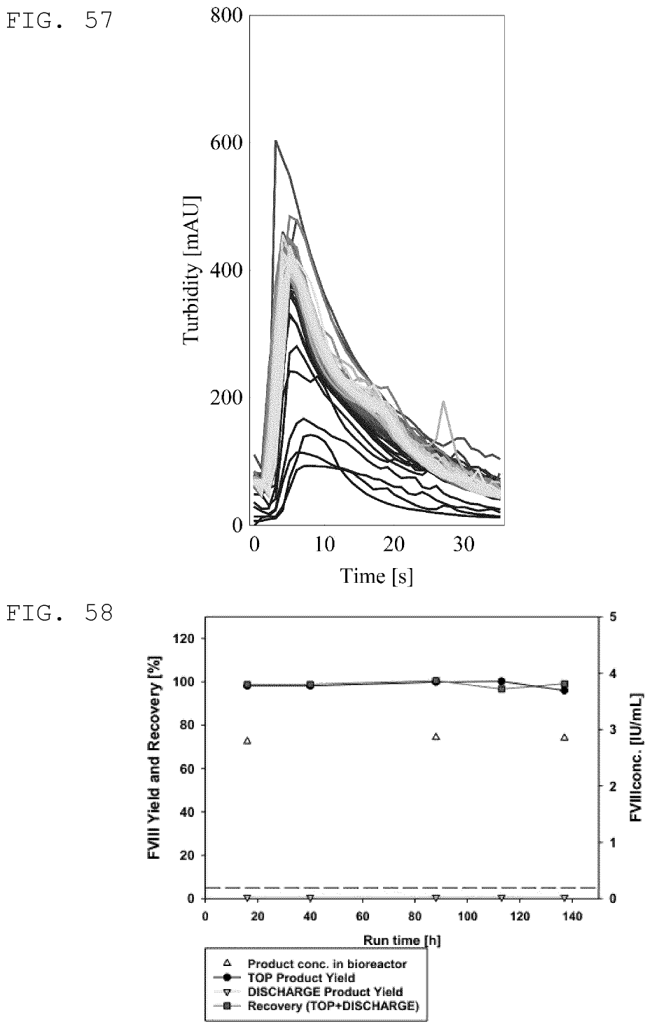

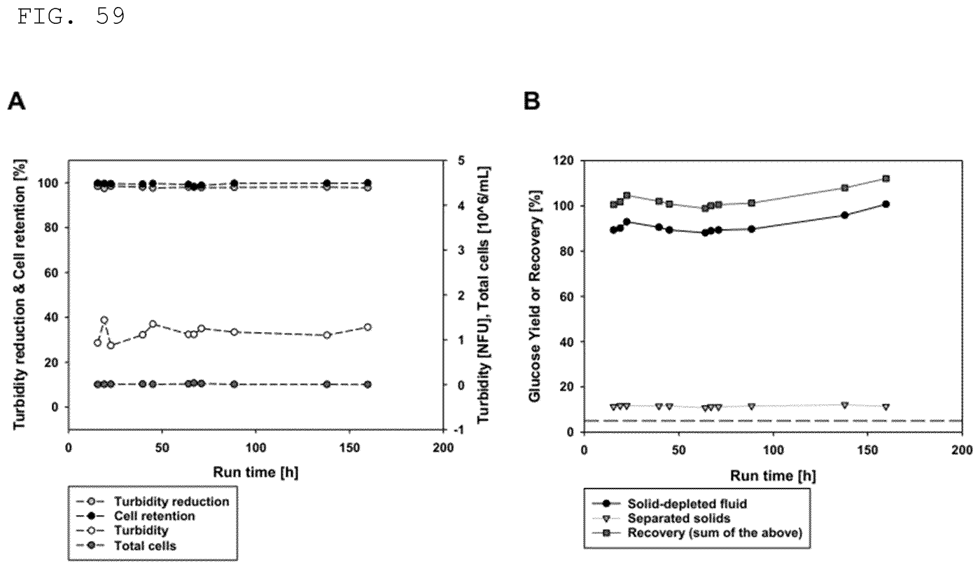

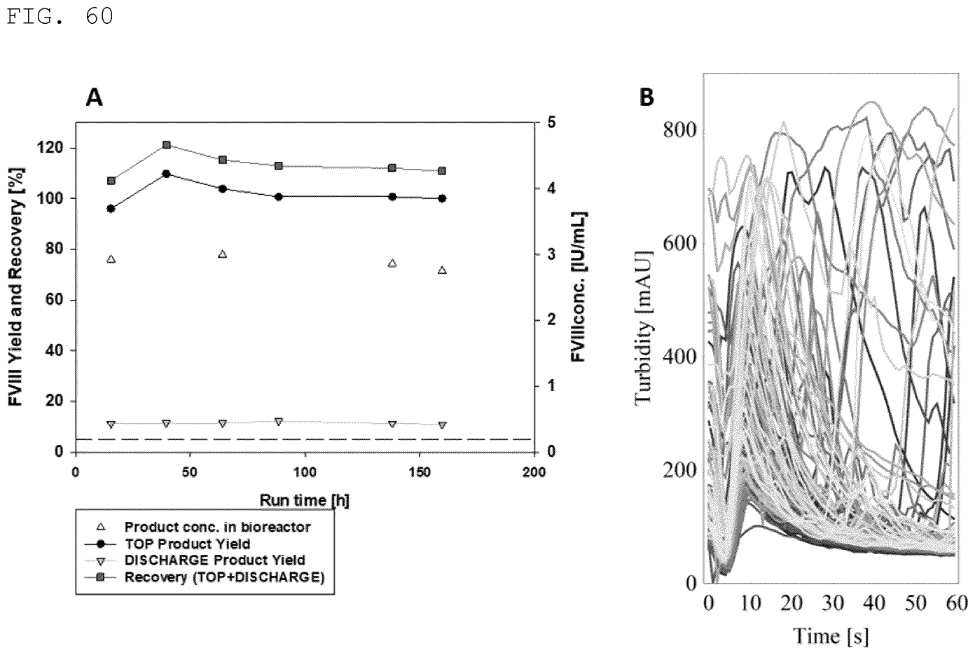

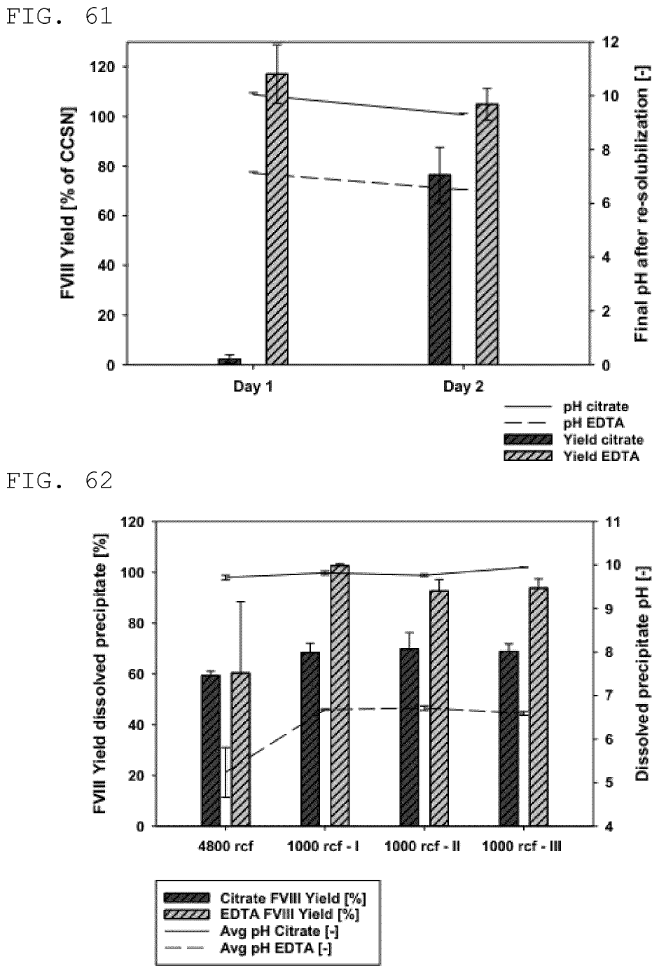

Two runs were performed using the structured bottom section recently developed. In the first run, the aim was to investigate the deposition of cells in the inclined plate settler. When the settling section is made from stainless steel, cell deposition cannot be monitored during a run. With the transparent, acrylic glass settling section used in this example, cell settling, deposition and sliding could be observed at any point during the run. Cells were found to be settling onto the plates with subsequent sliding and collection in the bottom section as expected. The shingle-type pattern of the sliding cells was preferentially occurring at the lower end of the settling section, where the majority of cells were removed. This cell removal run lasted for 137 h or almost 6 days, during which clarification and separation efficiency of the inclined settler were stable and no disturbances were encountered (FIG. 56). Stable operation is further supported by the consistency observed in the shape of the discharge peaks (FIG. 57A). Clarification efficiency was evaluated based on cell count and turbidity in the solid-depleted outflow collected at the top of the settler. Separation efficiency was evaluated by glucose measurement in the fractions collected from the top and bottom of the plate settler. Previous results had shown that glucose could be used as a surrogate marker for product. Under the experimental conditions, metabolic activity of the cells was reduced such that glucose was not metabolised within the inclined settler. The results obtained by glucose measurement were complemented by determination of FVIII activity in selected samples. Yield of FVIII in the top overflow was >96%, while it was below the limit of quantification (>0.2 IU/mL) in the fraction containing the removed cells (FIG. 58).

In the second run using the structured bottom section, the cell density in the bioreactor was increased from 1.5 to initially 3 and finally 6×10\hat{ }6 cells/mL. The aim of this experiment was to demonstrate the suitability of the structured bottom section for solid-liquid separation of cell suspensions with higher cell count. An increase in starting cell density of the suspension to be separated, resulted in an increase in the starting turbidity. As a consequence of higher initial turbidity, also the turbidity in the solid-depleted fluid increased over the duration of the run. The relative turbidity reduction, however, remained constant throughout the run and was independent of the starting turbidity (data not shown). In total, this cell removal run lasted for approx. 12 days. The operation was fully automated and highly stable during the entire run time. These results highlight the suitability of the unit operation for stable, long-term, continuous solid-liquid separation.

Cell Removal Using an Inclined Settler with a Conventional Bottom Section

In addition to the runs that were performed using the structured bottom section, a conventional, open bottom section was used in combination with the same acrylic glass settling section. With the conventional bottom section washing of collected solids is not possible. Hence, the collected solids remain suspended in their original fluid phase and thus, solid removal directly translates to loss of soluble product. Without prior optimization a discharge interval of 30 min at a volume of approx. 12 mL was used. Based on the feed flow-rate and the discharged volume within a given time window, this would translate to a product loss of 11.4% (yield=discharge volume [mL]/feed volume [mL]=discharge flow rate [mL/min]×discharge duration [min]/feed flow rate [mL/min]×discharge interval [min]).

This expectation was confirmed by the yield calculated from glucose offline measurements, where is was found to be 11.4% in average (FIG. 59B). The results that were based on glucose were supported by determination of FVIII activity in selected samples. These data also showed 11.4% FVIII yield in the collected solids. FVIII yield in the solid-depleted fluid was ˜100%, which resulted in a recovery of above 100% for FVIII (FIG. 60A). The efficiency of solid-liquid separation, i.e. cell removal, was not affected by the change in bottom section concept. Cell count and turbidity in the top overflow were reduced by >97% and remained highly stable throughout the entire experiment (FIG. 59A).

### Conclusions

Using an acrylic glass settling section, cell removal during the experiments was observed. These observations showed that the cells were sliding down the plates as expected. Furthermore, it was shown that the inclined settler is able to handle at least a 4× increase in cell density without adverse effects on the separation and clarification efficiency, provided the wash fluid density is adjusted to compensate for a higher feed suspension density. Comparison of the structured bottom section with a conventional bottom section, confirmed increased product loss in the absence of a wash step. The shape of the discharge peaks and thus the amount of cells collected in a cycle, fluctuated with the open bottom section, while it was highly stable with the structured bottom section.

### Example 12: EDTA as an Alternative to Citrate for Re-Solubilization

In some embodiments, the present invention includes a re-solubilization step of re-solubilizing the precipitated protein in accordance with the invention. This re-solubilization can be performed with citrate (see above). However, in this example EDTA was tested as an alternative to citrate for re-solubilization. Before that EDTA had been excluded as a potential candidate for re-solubilization, because its high complexing capability for calcium was assumed to be detrimental for FVIII activity.

To evaluate for incubation time and the re-solubilization agent as such, two precipitation experiments were performed on two consecutive days. The re-solubilized samples from the first day, were held at 4° C. until the next day and all samples were analysed for FVIII activity on the 2nd day. The data obtained in this experiment showed some difference in yield for samples re-solubilized using citrate, while yield after dissolution using EDTA was comparable (FIG. 61). The results also showed a distinct difference in pH in the re-solubilized samples, where the pH value of citrate dissolved samples was around 10 and pH for EDTA samples was between 7.1 and 6.5. The difference in pH may influence the final FVIII.

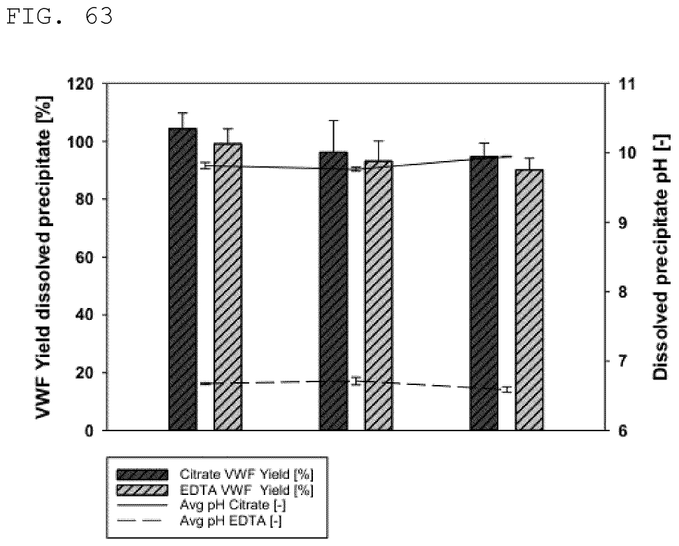

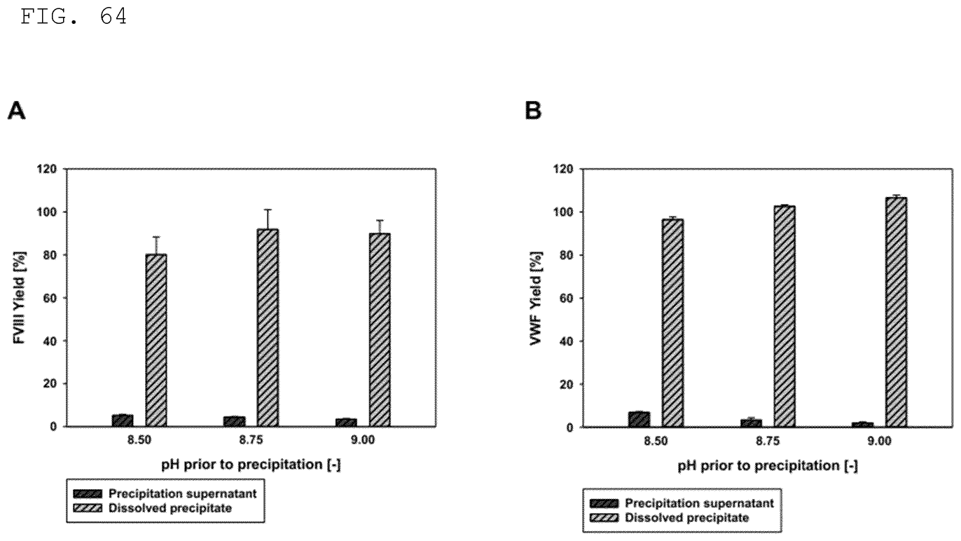

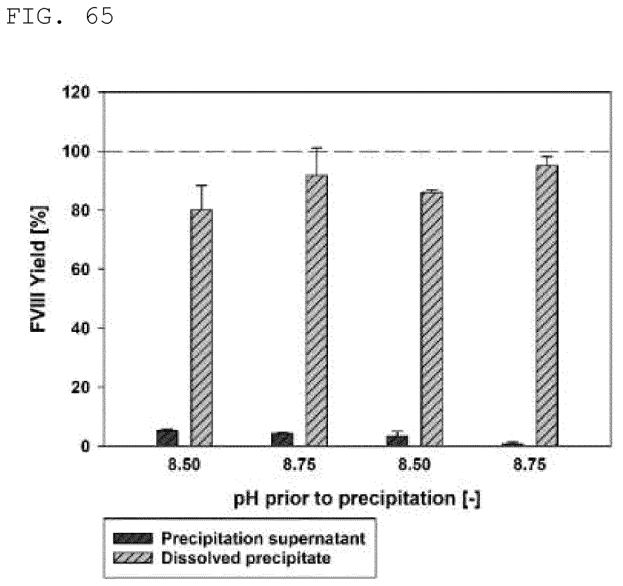

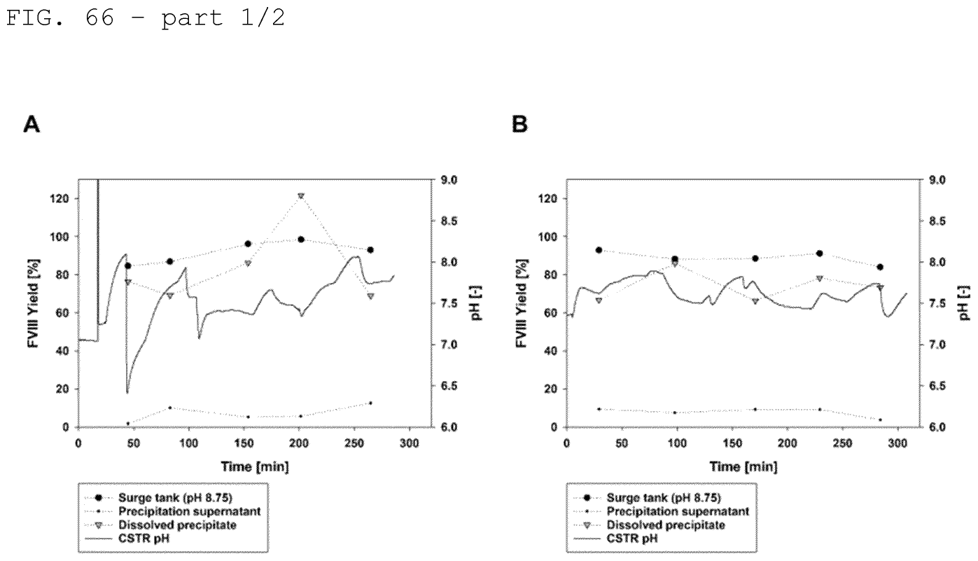

Because of the differences in the yield observed in the above experiment, reproducibility with citrate and EDTA re-solubilization was investigated. Using cell culture supernatant from different shipment that was stored for different periods of time at >−60° C., four precipitation experiments were performed on four consecutive days. The experimental conditions were held constant with one exception: The first experiment differed from the following three in the centrifugation force applied for solid-liquid separation. As a consequence, the re-solubilization of the precipitate was more difficult and required more re-solubilization agent. The pH after dissolution did not differ with centrifugation force for citrate, but was lower with the higher rcf-value for EDTA. The lower pH in the dissolved precipitate for EDTA also resulted in a decrease in yield. Notably, the larger variability in the pH after dissolution was also reflected by a larger variability in the corresponding yield values (FIG. 62). In the following three precipitation experiments, experimental conditions remained unchanged. By using a lower centrifugal force of 1000 g, stable yield values for citrate and EDTA could be obtained. Stable yield was accompanied by stable pH after re-solubilization. The final pH value using citrate was relatively high on all three four days at around pH 10 with a corresponding FVIII yield of 69.0±0.6%. When using EDTA for re-solubilization, the pH after dissolution as distinctively lower at ˜6.5, where the lower pH resulted in FVIII yield of 96.3±4.5%. In addition to FVIII Yield, VWF yield was determined for the same samples. For VWF there was neither a dependence on pH after re-solubilization nor a dependence on the used re-solubilization agent observed. VWF yield obtained with citrate was 98.4±4.2% and yield obtained with EDTA was 94.1±3.7% (FIG. 63). Thus, precipitation yield for VWF was highly stable under the experimental conditions. These results are based on VWF antigen content of the samples.

Thus, these experiments demonstrate that using EDTA for re-solubilization is advantageous compared to citrate in terms of higher FVIII yield. FVIII yield obtained with citrate was approx. 70%, while FVIII yield obtained with EDTA was >95% on three different days. With both re-solubilization agents thawed cell culture supernatant was precipitated in a highly reproducible manner (SD<5%).

### Example 13: Importance of pH for Calcium Phosphate Precipitation

The present invention includes a protein precipitation step of precipitating the protein in the fluid in accordance with the invention. Preferably, calcium phosphate is used as a precipitant in this step of the method of the invention. In this example the influence of the pH of the fluid in accordance with the present invention was investigated.

Differences in yield observed between some of the experiments described above may be due to the difference in starting material. With fresh cell culture supernatant (CCSN) a larger drop in pH was observed upon precipitation. Due to the pH dependence of calcium phosphate precipitation, a lower final pH may result in reduced precipitate formation, which in turn could cause lower yield of FVIII and VWF. Before testing this hypothesis in continuous mode, batch precipitation experiments were performed with fresh cell culture supernatant. The pH was adjusted to 8.5, 8.75 and 9.0 prior to precipitation. All other parameters, such as precipitant concentration, mixing, sample volume, centrifugation speed, re-solubilization agent, etc. remained identical to the conditions that were used during the reproducibility experiments described in Example 12. For these conditions stable yield using different freeze-thawed CCSN batches has been established in Example 12. A difference between these results and the ones obtained with fresh material would thus be attributed to the freeze-thaw cycle. The results obtained with fresh CCSN showed an increase of FVIII from 80.1 to 91.8%, when the pH prior to precipitation was increased from 8.5 to 8.75 (FIG. 64A). The residual FVIII concentration (i.e. activity) in the precipitation supernatant did not change distinctively with pH. FVIII precipitation supernatant yield was 5.2% at pH 8.5 prior to precipitation and decreased to 3.5% at pH 9.0. For VWF the yield in the dissolved precipitate increased steadily with pH, where the highest value was 106.6%. A yield above 100% is attributed to the error of the antigen-ELISA. VWF yield in the precipitation supernatant decreased from 6.8% at pH 8.5 to 2.0% at pH 9.0. Based on protein yield, the highest pH value appeared most favourable. However, taking into account the consumption of EDTA required for re-solubilization and the concentration factor (or volume reduction) achieved, the intermediate pH balances yield and concentration. The concentration factor was highest with pH 8.5 and lowest at pH 9.0 (Table 20).

TABLE 20
Observed pH values in the precipitate suspension and after re-
solubilization, EDTA consumption and final concentration factor
achieved for different setpoints for pH prior to precipitation.

| Setpoint pH | pH | | | |
|---|---|---|---|---|
| prior to | precipitate | pH after re- | | Final |
| precipitation | suspension | solubilization | consumption | concentration |
| [—] | [—] | [—] | [g/g CCSN] | factor [—] |
| 8.5 | 8.03 | 6.50 | 0.018 | 11.4 |
| 8.75 | 8.13 | 6.39 | 0.024 | 10.3 |
| 9.0 | 8.25 | 6.21 | 0.031 | 9.4 |

Based on the previous batch precipitation experiments with fresh CCSN a yield for VWF was expected to be around 80%, which was exceeded in this experiment. However, during the experiments performed previously, the pH in the precipitate suspension (CSTR pH) had been 7.75 (starting pH 8.5). In the latest experiments using fresh CCSN, the lowest pH in the precipitate suspension was 8.03. When thawed CCSN had been used at different starting pH values the pH in the precipitate suspension was 8.14, where the starting pH was 8.5. The pH values obtained with thawed CCSN as obtained previously are reproduced below in Table 21. The results indicate that the pH after precipitation is critical for achieving high yield. While product yield was reduced at pH 7.75 and 8.0 (after precipitation), it was found to be >90% for both proteins, when a final pH of approx. 8.15 was achieved. These results also support the notion that a compensation of the difference in starting material is in fact possible.

TABLE 21
pH values observed during previous experiments.

| | | pH after | |
|---|---|---|---|
| Starting pH | Buffer species | precipitation | Delta pH |
| 8.5* | 50 mM TRIS | 8.84 | +0.34 |
| 8.0 | n.a. | 7.94 | −0.06 |
| 8.5 | n.a. | 8.14 | −0.36 |
| 9.0 | n.a. | 8.35 | −0.65 |

In experiments with fresh material

| | | | |
|---|---|---|---|
| 8.5 | n.a. | 7.75 | −0.75 |

Starting pH = target pH for pH modification, where pH of Tris modified sample was calculated and not measured.
Buffer species = concentration and name of buffering species.
n.a. = not applicable.
pH after precipitation.
Delta pH = “pH after precipitation” − “starting pH”.

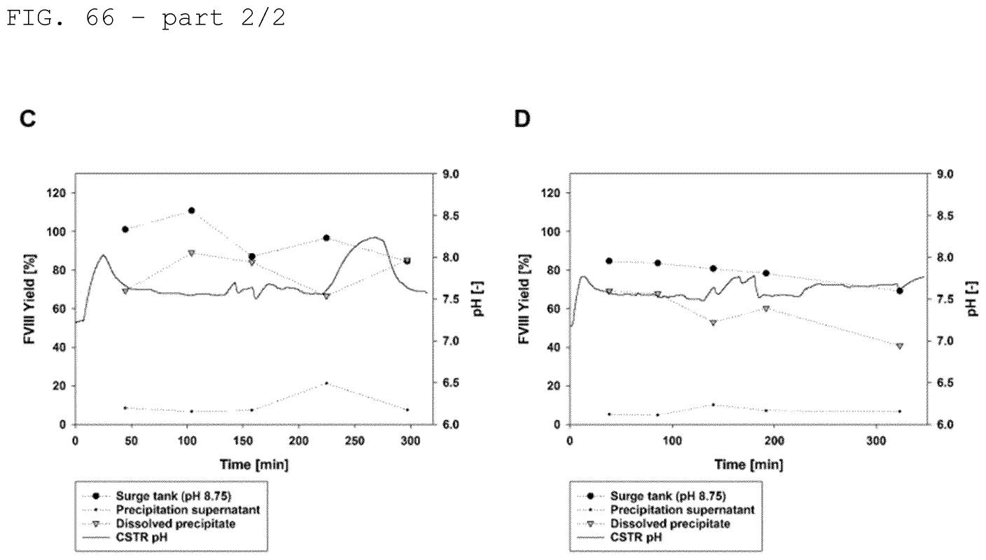

The above batch precipitation experiment with fresh harvest material suggested an increase in product yield by ˜10% by increasing the starting pH from 8.5 to 8.75. This experiment was repeated at the relevant pH values to confirm the results. The resulting data was comparable to the previous findings. FIG. 65 shows the previous results in the left half of the plot and the new results on the right-hand. The difference between the replicates performed on different days was <6%, which is within the expected range. The relative difference between the tested pH values was confirmed by the second replicate. By increasing the pH setpoint from 8.5 to 8.75 FVIII yield could be increased from 80 to 92% in the first and from 86 to 95% in the second precipitation experiment. In all cases, there were small residual amounts of FVIII in the precipitation supernatant.

### Example 14: Continuous Precipitation with Optimized Parameters

### Materials and Methods

### Continuous Precipitation of Cell Culture Supernatant

Continuous precipitation was performed using basically the automated setup that was also used for automated continuous precipitation described above. The operation parameters are also described in detail above (examples 7 to 10, material and methods). The setup was slightly modified, by removing the tubular reactor and replacing it by an open piece of tubing. The settings for the target pH and the action limits were modified in the custom software to adjust to pH 8.75 instead of 8.5 (see Table 22). Samples were taken from the surge tank and at the outlet of the CSTR.

TABLE 22
Set values of pH control parameters.

| pH control parameter | Set value |
|---|---|
| Nominal flow [mL/min] | 0.04 |
| p-value [—] | 0.3 |
| Critical pH [—] | 8.5 |
| Lower limit pH [—] | 8.7 |
| Target pH [—] | 8.73 |
| Upper Limit pH [—] | 8.74 |

### Solid-Liquid Separation and Re-Solubilization

Separation of the precipitate from the precipitation supernatant was performed by centrifugation at 1000 g, 10 min, 4° C. Centrifugation was performed using a Heraeus Multifuge X3 FR swing-out rotor centrifuge (Thermo Scientific). The collected precipitate was re-solubilised after re-suspension in 3 mL TBS-T buffer after centrifugation. Re-solubilisation was achieved by step-wise addition of 0.1 M EDTA until complete dissolution was reached.

### Results

The continuous precipitation process was performed with the optimized starting pH value (i.e. the pH setpoint prior precipitation, see Example 13) and EDTA as optimized re-solubilization agent (see Example 12). Four continuous precipitation experiments were performed with these optimized precipitation and re-solubilization conditions.

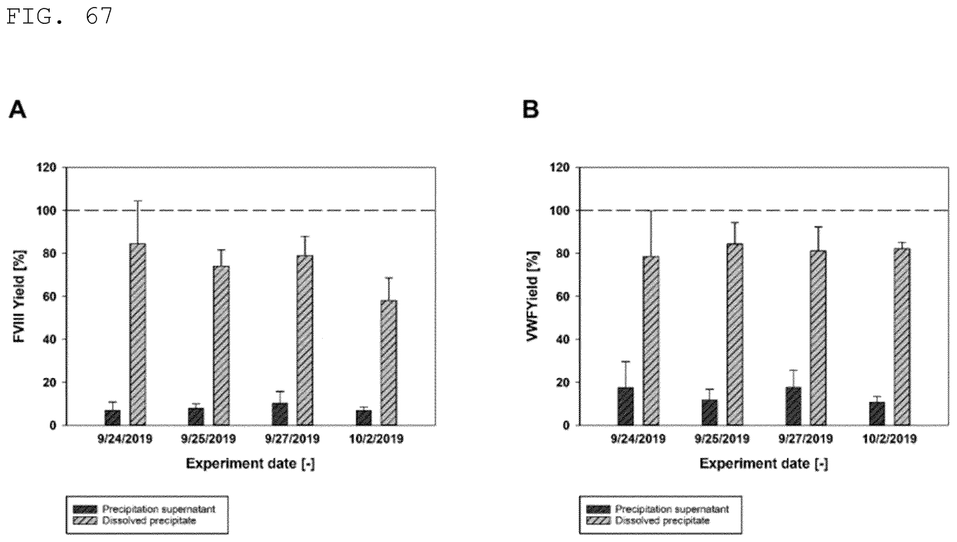

The pH observed in the precipitate suspension was overlaid with the FVIII yield results in the plots shown in FIG. 66. FVIII yield after pH modification was stable and high in the first three experiments. In the fourth experiment it was still stable, but reduced at around 80%. A similar pattern was also observed for FVIII yield in the dissolved precipitate, which is summarized in FIG. 67A. While, the yield ranged between 74 and 84% in the first three experiments, it was 58% in the last experiment. VWF yield was comparable for all four experiments. In addition to the yield data, Table 23 also lists the average pH in the re-solubilized samples measured offline after re-solubilization. The last continuous precipitation experiment, in which FVIII yield was lower, also exhibited a lower average pH in the re-solubilized samples. Similar effects had previously been observed during the reproducibility study of Example 12. In line with these previous findings there was no dependence of VWF yield on pH in the latest experiments.

TABLE 23
Average yield for FVIII, VWF and pH found in five
samples taken during continuous precipitation of fresh
harvest, plus average over all four experiments
and corresponding standard deviation (SD).

| | Repli- | Repli- | Repli- | Repli- | | |
|---|---|---|---|---|---|---|
| | cate | cate | cate | cate | | |
| Sample ID | 1 | 2 | 3 | 4 | Average | SD |

FVIII

| | | | | | | |
|---|---|---|---|---|---|---|
| Prec. SN | 7.0 | 7.8 | 10.2 | 6.8 | 8.0 | 1.3 |
| Diss. Prec | 84.5 | 74.1 | 78.9 | 58.2 | 73.9 | 9.8 |

VWF

| | | | | | | |
|---|---|---|---|---|---|---|
| Prec. SN | 17.4 | 11.7 | 17.7 | 10.8 | 14.4 | 3.2 |
| Diss. Prec. | 78.6 | 84.3 | 81.2 | 82.2 | 81.6 | 2.1 |

pH data

| | | | | | | |
|---|---|---|---|---|---|---|
| pH after re- | 6.38 | 6.19 | 6.31 | 5.77 | 6.16 | 0.24 |
| solubilization | | | | | | |

In summary, two changes were implemented to the continuous precipitation process described in the previous examples. The first change concerned the starting pH, which was increased from 8.5 to 8.75 and the second change concerned the re-solubilization agent, which was changed from citrate to EDTA. By making these adaptations to the precipitation process, the FVIII yield in the continuous process could be increased from 56 to 74% (compare Table 24). VWF yield remained constant. As a consequence, it is expected that FVIII yield increases by the same number of percent points in the continuous precipitate collection (prediction in italic letters in Table 24).

TABLE 24
Summary of product yield throughout the process. Product concentration
in the bioreactor was assumed as 100%. The yield values indicate
the sum of all steps up until a given unit operation. Improved
precipitate collection yield is an estimation based on the
improved continuous precipitation results.

| | | | | Improved | |
|---|---|---|---|---|---|
| | | Contin- | | contin- | Improved |
| | | uous | Precip- | uous | precip- |
| Unit | Cell | precip- | itate | precip- | itate |
| operation | removal | itation | collection | itation | collection* |
| FVIII Yield | 98 | 56 | 33 | 74 | >51 |
| [%] | | | | | |
| VWF Yield | 100 | 85 | 56 | 82 | 56 |
| [%] | | | | | |

### INDUSTRIAL APPLICABILITY

The method for continuous recovering of a protein from a fluid in accordance with the present invention can be used to recover various commercially useful proteins, such as biopharmaceutical drugs. Such biopharmaceutical drugs can be formulated as pharmaceutical compositions in accordance with the method for producing a pharmaceutical composition in accordance with the present invention. The inclined plate settler of the present invention can be used in the method for continuous recovering of a protein from a fluid in accordance with the present invention. Thus, the present invention is industrially applicable.

### REFERENCES

(1) Warikoo, V. & Godawat, R. A new use for existing technology—continuous precipitation for purification of recombination proteins. Biotechnology Journal 10 (2015).
(2) Velayudhan, A. Continuous antibody purification using precipitation: An important step forward. Biotechnology Journal 9, 717-718 (2014).
(3) Martinez, M., Spitali, M., Norrant, E. L. & Bracewell, D. G. Precipitation as an Enabling Technology for the Intensification of Biopharmaceutical Manufacture. Trends Biotechnol (2018).
(4) Hammerschmidt, N., Hintersteiner, B., Lingg, N. & Jungbauer, A. Continuous precipitation of IgG from CHO cell culture supernatant in a tubular reactor. Biotechnology Journal 10, 1196-1205 (2015).
(5) Hammerschmidt, N., Hobiger, S. & Jungbauer, A. Continuous polyethylene glycol precipitation of recombinant antibodies: Sequential precipitation and resolubilization. Process Biochemistry 51, 325-332 (2016).
(6) Burgstaller, D., Jungbauer, A. & Satzer, P. Continuous integrated antibody precipitation with two-stage tangential flow microfiltration enables constant mass flow. Biotechnol Bioeng 116, 1053-1065 (2019).
(7) Kateja, N., Agarwal, H., Saraswat, A., Bhat, M. & Rathore, A. S. Continuous precipitation of process related impurities from clarified cell culture supernatant using a novel coiled flow inversion reactor (CFIR). Biotechnology Journal 11, 1320-1331 (2016).
(8) Gagnon, P. Technology trends in antibody purification. Journal of chromatography. A 1221, 57-70 (2012).
(9) Blumhoff, M., Steiger, M. G., Marx, H., Mattanovich, D. & Sauer, M. Six novel constitutive promoters for metabolic engineering of Aspergillus niger. Appl Microbiol Biotechnol 97, 259-267 (2013).
(10) Steiger, M. G., Rassinger, A., Mattanovich, D. & Sauer, M. Engineering of the citrate exporter protein enables high citric acid production in Aspergillus niger. Metabolic Engineering 52, 224-231 (2019).
(11) Satzer, P., Tschelieβnigg, A., Sommer, R. & Jungbauer, A. Separation of recombinant antibodies from DNA using divalent cations. Engineering in Life Sciences 14, 477-484 (2014).
(12) Sauer, D. G. et al. A two-step process for capture and purification of human basic fibroblast growth factor from E. coli homogenate: Yield versus endotoxin clearance. Protein Expr Purif 153, 70-82 (2019).
(13) Pham, L., Ye, H., Cosset, F.-L., Russell, S. J. & Peng, K.-W. Concentration of viral vectors by co-precipitation with calcium phosphate. The Journal of Gene Medicine 3, 188-194 (2001).
(14) Ko, H. F. & Bhatia, R. Evaluation of Single-Use Fluidized Bed Centrifuge System for Mammalian Cell Harvesting. Biopharm Int 25, 34-40 (2012).

1. Method for continuous recovering of a protein from a fluid, wherein the method comprises the following steps:
a protein precipitation step of precipitating the protein in the fluid; and
a protein separation step of separating the precipitated protein from the fluid;
wherein all steps are performed in an integrated process.

2. The method of claim 1, wherein all steps of the method are performed continuously.

3. The method of claim 1 or claim 2, wherein the protein has a molecular weight of 250 kDa or more, preferably wherein the protein has a molecular weight of 500 kDa or more.

4. The method of any one of claims 1 to 3, wherein before the protein precipitation step the concentration of the protein in the fluid comprising the protein is below 20 μg/ml, preferably between 0.05 μg/ml and 20 μg/ml.

5. The method of any one of claims 1 to 4, wherein the protein is a blood coagulation factor.

6. The method of claim 5, wherein the protein is Factor VIII.

7. The method of any one of claims 1 to 4, wherein the protein is von Willebrand factor.

8. The method of any one of claims 1 to 4, wherein the protein is a protein complex comprising Factor VIII and von Willebrand factor.

9. The method of any one of claims 1 to 8, wherein in the protein precipitation step the protein is precipitated using a precipitant.

10. The method of claim 9, wherein the precipitant is selected from the group consisting of calcium phosphate, polyethylene glycol (PEG), an affinity ligand, a pH modifying agent, an organic solvent such as ethanol or acetone, a polyelectrolyte such as polyacrylic acid or polyethylenimine, and a salt.

11. The method of claim 9 or 10, wherein the precipitant comprises phosphate.

12. The method of any one of claims 9 to 11, wherein the precipitant is calcium phosphate, magnesium phosphate, or zinc phosphate.

13. The method of any one of claims 9 to 12, wherein the precipitant is calcium phosphate.

14. The method of claim 13, wherein the protein precipitation step comprises adding calcium ions to the fluid.

15. The method of claim 14, wherein calcium ions are added to a final concentration of between 10 mM and 50 mM, preferably between 10 mM and 30 mM.

16. The method of claim 14, wherein calcium ions are added to a final concentration of between 10 mM and 20 mM, preferably about 15 mM.

17. The method of any one of claims 13 to 16, wherein the protein precipitation step comprises adding phosphate ions to the fluid.

18. The method of claim 17, wherein phosphate ions are added to a final concentration of between 1 mM and 10 mM, preferably between 1 mM and 5 mM.

19. The method of claim 17, wherein phosphate ions are added to a final concentration of between 1 mM and 3 mM, preferably about 2 mM.

20. The method of any one of claims 9 to 19, wherein the protein precipitation step comprises mixing the fluid comprising the protein and the precipitant.

21. The method of claim 20, wherein mixing is performed in at least one reactor selected from the list consisting of a continuous stirred tank reactor (CSTR), a tubular reactor (TR), a segmented flow reactor, and an impinging jet reactor.

22. The method of claim 20 or 21, wherein mixing is performed in a continuous stirred tank reactor (CSTR).

23. The method of any one of claims 1 to 22, wherein the pH of the fluid before precipitating the protein is adjusted to a pH of between 8.5 and 9.0, preferably to a pH of about 8.75.

24. The method of any one of claims 1 to 23, wherein the pH of the fluid after precipitating the protein is between 6 and 7.5, preferably between 6.5 and 7, most preferably about 6.5.

25. The method of any one of claims 1 to 24, wherein in the protein separation step a plate settler for protein separation, continuous tangential flow filtration, or fluidized bed centrifugation is used for separating the precipitated protein from the fluid.

26. The method of any one of claims 1 to 25, wherein in the protein separation step a plate settler for protein separation is used for separating the precipitated protein from the fluid.

27. The method of claim 26, wherein the plate settler for protein separation is an inclined plate settler with a lower portion, an upper portion, and at least one sedimentation channel for letting the precipitated protein settle, said sedimentation channel extend from the lower portion to the upper portion,
the inclined plate settler being configured to be oriented during use such that the at least one sedimentation channel extends from the lower portion to the upper portion in a direction that is inclined with respect to the direction of gravity,
wherein the at least one sedimentation channel is connected to a fluid outlet for draining a rest fluid at the upper portion.

28. The method of claim 27, wherein the length of the sedimentation channel is between 20 cm and 150 cm, preferably between 20 cm and 100 cm, more preferably between 20 cm and 80 cm, more preferably between 30 cm and 70 cm, more preferably between 40 cm and 60 cm, most preferably about 50 cm.

29. The method of claim 27 or 28, wherein the at least one sedimentation channel of the plate settler for protein separation is connected to a bottom section, wherein the bottom section comprises at least one inlet channel for feeding the fluid comprising the precipitated protein to the plate settler, and at least one collection channel for collecting the settled precipitated protein descending from the at least one sedimentation channel,
wherein said at least one inlet channel and said at least one collection channel are fluidly separated from each other, said inlet channel and said collection channel being connected to said at least one sedimentation channel, to form fluid connections between said at least one inlet channel and said at least one sedimentation channel and between said at least one collection channel and said at least one sedimentation channel, respectively.

30. The method of claim 29, wherein the bottom section that is connected to the plate settler for protein separation further comprises at least one wash fluid supply channel for supplying a wash fluid to one sedimentation channel or to one collection channel, said at least one wash fluid supply channel being fluidly separated from other wash fluid supply channels and from all inlet channels.

31. The method of claim 30, wherein the at least one wash fluid supply channel and the at least one collection channel corresponding to the same sedimentation channel of the plate settler for protein separation are fluidly connected by an opening in a wall portion shared by said wash fluid supply channel and said collection channel.

32. The method of claim 30 or claim 31, wherein the fluid comprising the precipitated protein is supplied to the bottom section, which is connected to the plate settler for protein separation, through the at least one inlet channel, and a wash fluid is supplied through the at least one wash fluid supply channel,
wherein the density of the wash fluid is higher than the density of the fluid comprising the precipitated protein, and
wherein the rest fluid is drained through the fluid outlet at the upper portion and the settled precipitated protein is drained through the collection channel.

33. The method of claim 32, wherein the density of the wash fluid is between 0.3% and 1.5% higher than the density of the fluid comprising the precipitated protein, preferably between 0.55% and 1.20% higher than the density of the fluid comprising the precipitated protein.

34. The method of claim 32 or 33, wherein the wash fluid comprises Tris and sodium chloride.

35. The method of claim 34, wherein the wash fluid comprises Tris at a concentration of about 2 mM and sodium chloride at a concentration of about 272 mM.

36. The method of claim 34 or 35, wherein the wash fluid further comprises calcium chloride.

37. The method of claim 36, wherein the wash fluid comprises calcium chloride at a concentration of between 4 mM and 12 mM.

38. The method of claim 36 or 37, wherein the wash fluid comprises Tris at a concentration of about 2 mM, sodium chloride at a concentration of about 231 mM and calcium chloride at a concentration of about 12 mM.

39. The method of any one of claims 32 to 38, wherein the wash fluid has a pH of 7.5 or higher, preferably of 8 or higher, most preferably of about 8.25.

40. The method of any one of claims 32 to 39, wherein the wash fluid is supplied through the at least one wash fluid supply channel and the settled precipitated protein is drained through the collection channel at regular intervals.

41. The method of claim 40, wherein the wash fluid is supplied through the at least one wash fluid supply channel and the settled precipitated protein is drained through the collection channel at regular intervals of between 15 min and 45 min, preferably about 30 min.

42. The method of any one of claims 32 to 41, wherein the wash fluid is supplied through the at least one wash fluid supply channel and the settled precipitated protein is drained through the collection channel at a volumetric flow rate of about 20 to 60 mL/min, preferably about 40 mL/min.

43. The method of any one of claims 1 to 42, wherein the method further comprises the following steps before the protein precipitation step:
a protein production step of culturing cells in a fluid, wherein the cells produce the protein and release the protein into the fluid; and
a cell separation step of separating the cells from the fluid comprising the protein.

44. The method of claim 43, wherein the cells are mammalian cells.

45. The method of claim 44, wherein the cells are CHO cells.

46. The method of any one of claims 43 to 45, wherein the fluid is a cell culture medium.

47. The method of any one of claims 43 to 46, wherein in the protein production step the cells are cultured in a perfusion reactor or a chemostat reactor, preferably in a chemostat reactor.

48. The method of any one of claims 43 to 47, wherein in the cell separation step a plate settler for cell separation is used for separating the cells from the fluid comprising the protein.

49. The method of claim 48, wherein the plate settler for cell separation is an inclined plate settler with a lower portion, an upper portion, and at least one sedimentation channel for letting the cells settle, said sedimentation channel extend from the lower portion to the upper portion,
the inclined plate settler being configured to be oriented during use such that the at least one sedimentation channel extends from the lower portion to the upper portion in a direction that is inclined with respect to the direction of gravity,
wherein the at least one sedimentation channel is connected to a fluid outlet for draining a rest fluid at the upper portion.

50. The method of claim 49, wherein the at least one sedimentation channel of the plate settler for cell separation is connected to a bottom section, wherein the bottom section comprises at least one inlet channel for feeding the fluid comprising the cells and the protein to the plate settler, and at least one collection channel for collecting the settled cells descending from the at least one sedimentation channel,
wherein said at least one inlet channel and said at least one collection channel are fluidly separated from each other, said inlet channel and said collection channel being connected to said at least one sedimentation channel, to form fluid connections between said at least one inlet channel and said at least one sedimentation channel and between said at least one collection channel and said at least one sedimentation channel, respectively.

51. The method of claim 50, wherein the bottom section that is connected to the plate settler for cell separation further comprises at least one wash fluid supply channel for supplying a wash fluid to one sedimentation channel or to one collection channel, said at least one wash fluid supply channel being fluidly separated from other wash fluid supply channels and from all inlet channels.

52. The method of claim 51, wherein the at least one wash fluid supply channel and the at least one collection channel corresponding to the same sedimentation channel of the plate settler for cell separation are fluidly connected by an opening in a wall portion shared by said wash fluid supply channel and said collection channel.

53. The method of claim 51 or 52, wherein the fluid comprising the cells and the protein is supplied to the bottom section, which is connected to the plate settler for cell separation, through the at least one inlet channel, and a wash fluid is supplied through the at least one wash fluid supply channel,
wherein the density of the wash fluid is higher than the density of the fluid comprising the cells and the protein, and
wherein the settled cells are drained through the collection channel and the rest fluid comprising the protein is drained through the fluid outlet at the upper portion.

54. The method of claim 53, wherein the wash fluid is supplied through the at least one wash fluid supply channel and the settled cells are drained through the collection channel at regular intervals.

55. The method of claim 53 or 54, wherein the wash fluid is supplied through the at least one wash fluid supply channel and the settled cells are drained through the collection channel at regular intervals of 5 min to 90 min.

56. The method of claim 53 or 54, wherein the wash fluid is supplied through the at least one wash fluid supply channel and the settled cells are drained through the collection channel at regular intervals of 15 min to 85 min.

57. The method of claim 53 or 54, wherein the wash fluid is supplied through the at least one wash fluid supply channel and the settled cells are drained through the collection channel at regular intervals of 25 min to 80 min.

58. The method of claim 53 or 54, wherein the wash fluid is supplied through the at least one wash fluid supply channel and the settled cells are drained through the collection channel at regular intervals of 35 min to 75 min.

59. The method of claim 53 or 54, wherein the wash fluid is supplied through the at least one wash fluid supply channel and the settled cells are drained through the collection channel at regular intervals of 45 min to 70 min.

60. The method of claim 53 or 54, wherein the wash fluid is supplied through the at least one wash fluid supply channel and the settled cells are drained through the collection channel at regular intervals of 55 min to 65 min.

61. The method of claim 53 or 54, wherein the wash fluid is supplied through the at least one wash fluid supply channel and the settled cells are drained through the collection channel at regular intervals of about 60 min.

62. The method of any one of claims 53 to 61, wherein the wash fluid is supplied through the at least one wash fluid supply channel and the settled cells are drained through the collection channel at a volumetric flow rate of between 50 to 70 mL/min, preferably about 60 mL/min.

63. The method of any one of claims 1 to 62, wherein the method further comprises the following step after the protein separation step:
a re-solubilization step of re-solubilizing the precipitated protein.

64. The method of claim 63, wherein in the re-solubilization step the precipitated protein is re-solubilized using citrate or EDTA.

65. The method of claim 64, wherein in the re-solubilization step the precipitated protein is re-solubilized using EDTA.

66. The method of claim 65, wherein in the re-solubilization step the precipitated protein is re-solubilized using EDTA at a final concentration of between 10 mM to 50 mM.

67. The method of claim 65 or 66, wherein in the re-solubilization step the precipitated protein is re-solubilized using EDTA at a final concentration of between 20 mM to 30 mM.

68. The method of any one of claims 65 to 67, wherein in the re-solubilization step the precipitated protein is re-solubilized using EDTA at a final concentration of about 25 mM.

69. The method of any one of claims 1 to 68, wherein the protein is a biopharmaceutical drug.

70. Recovered protein that is obtainable by the method of any one of claims 1 to 69.

71. Method for producing a pharmaceutical composition, comprising performing the method of claim 69 and formulating the recovered biopharmaceutical drug as a pharmaceutical composition.

72. Pharmaceutical composition that is obtainable by the method of claim 71.

73. An inclined plate settler for separating a solid component from a fluid, wherein the plate settler comprises a lower portion, an upper portion, and at least one sedimentation channel for letting the solid component settle, said sedimentation channel extend from the lower portion to the upper portion,
the plate settler being configured to be oriented during use such that the at least one sedimentation channel extends from the lower portion to the upper portion in a direction that is inclined with respect to the direction of gravity,
wherein the at least one sedimentation channel is connected to a fluid outlet for draining a rest fluid at the upper portion and connected to a bottom section at the lower portion,
wherein the bottom section comprises at least one inlet channel for feeding a fluid comprising the solid component to be separated to the plate settler, and at least one collection channel for collecting a settled component descending from the at least one sedimentation channel,
wherein said at least one inlet channel and said at least one collection channel are fluidly separated from each other, said inlet channel and said collection channel being connected to said at least one sedimentation channel, to form fluid connections between said at least one inlet channel and said at least one sedimentation channel and between said at least one collection channel and said at least one sedimentation channel, respectively,
wherein the bottom section further comprises at least one wash fluid supply channel for supplying a wash fluid to one sedimentation channel or to one collection channel, said at least one wash fluid supply channel being fluidly separated from other wash fluid supply channels and from all inlet channels, and
wherein the length of the sedimentation channel is between 20 cm and 150 cm, preferably between 20 cm and 100 cm, more preferably between 20 cm and 80 cm, and most preferably between 30 cm and 70 cm.

74. The inclined plate settler of claim 73, wherein the length of the sedimentation channel is between 40 cm and 60 cm, preferably about 50 cm.

75. The inclined plate settler of claim 73 or 74, wherein the solid component is a precipitated protein, preferably a precipitated protein complex comprising Factor VIII and von Willebrand factor.

76. The inclined plate settler of any one of claims 73 to 75, wherein the inclined plate settler contains a precipitated protein complex comprising Factor VIII and von Willebrand factor.
